(19) United States
(12) Patent Application Publication
(10) Pub. No.: US 2023/0210759 A1
(43) Pub. Date: Jul. 6, 2023

(54) METHODS AND MATCHA GREEN TEA CONTAINING COMPOSITIONS FOR PREVENTING AND TREATING TOOTH EROSION

(71) Applicant: University of Tennessee Research Foundation, Knoxville, TN (US)

(72) Inventors: Mojdeh Dehghan, Germantown, TN (US); Daranee Versluis, Memphis, TN (US); Hassan Almoazen, Bartlett, TN (US)

(73) Assignee: University of Tennessee Research Foundation, Knoxville, TN (US)

(21) Appl. No.: 18/009,579

(22) PCT Filed: Jun. 9, 2021
(86) PCT No.: PCT/US2021/036603

§ 371 Date: Dec. 9, 2022

Related U.S. Application Data
(60) Provisional application No. 63/036,719, filed on Jun. 9, 2020.

(51) Int. Cl. A61K 8/9789; A61Q 11/00; A61K 8/02; A61K 8/19

(52) U.S. Cl. CPC A61K 8/9789; A61Q 11/00; A61K 8/0216; A61K 8/19; A61K 2800/92; A61K 2800/74

(57) ABSTRACT

An oral composition in the form of a lozenge or lollipop is provided. The composition comprises crosslinked polyvinylpyrrolidine (PVP), xylitol and Matcha green tea. A multi-layer lozenge or lollipop for neutralizing saliva and re-hardening tooth enamel is also provided. The lozenge or lollipop comprises an alkalinizing agent; a re-mineralizing agent comprising Matcha green tea; a base; a plasticizer; and a sugar alcohol, wherein the lozenge or lollipop upon oral administration raises the saliva pH from about 7 to about 10 in about 1 minute. Methods of making and treatment are also provided.

### BACKGROUND

Dental care is one of the primary health concerns of people worldwide. The main diseases of teeth include plaque, dental carries and periodontitis. Dental caries are caused by dissolution of the mineral portion of the tooth, which can result in pain and loss of viability of the tooth, necessitating costly repair or extraction of the tooth. When bacteria in the mouth combine with sugars, they produce acid. This acid can erode tooth enamel and damage our teeth. Fluoride can protect teeth from demineralization that is caused by the acid. If acid has already caused some damage to the teeth, fluoride accumulates in the demineralized areas and can begin strengthening the enamel. This constitutes remineralization.

Clean teeth do not decay easily. However, even with vigorous cleaning it is difficult to keep teeth sufficiently clean. Various methods have been developed to prevent or alleviate dental caries including, for example, the addition of sodium fluoride, sodium silicofluoride or hydrofluosilicic acid to drinking water, and sodium fluoride or tin fluoride to topical preparations, including dentifrices and mouth rinses.

More recently, concerns have arisen regarding fluoride's effect on health, including problems with bones, teeth, and neurological development. Excessive exposure to fluoride has been linked to a number of health issues. For example, exposure to high concentrations of fluoride during childhood, when teeth are developing, can result in mild dental fluorosis. Excess exposure to fluoride can lead to a bone disease known as skeletal fluorosis. Over many years, this can result in pain and damage to bones and joints. The bones may become hardened and less elastic, increasing the risk of fractures. If the bones thicken and bone tissue accumulates, this can contribute to impaired joint mobility. In some cases, excess fluoride can damage the parathyroid gland. This can result in hyperparathyroidism, which involves uncontrolled secretion of parathyroid hormones. This can result in a depletion of calcium in bone structures and higher-than-normal concentrations of calcium in the blood. Lower calcium concentrations in bones make them more susceptible to fractures. Other health problems resulting from an increased exposure to fluoride include acne and other skin problems, cardiovascular problems, including arteriosclerosis and arterial calcification, high blood pressure, myocardial damage, cardiac insufficiency, and heart failure, reproductive issues, such as lower fertility and early puberty in girls, thyroid dysfunction, conditions affecting the joints and bones, such as osteoarthritis, bone cancer, and temporomandibular joint disorder (TMJ), neurological problems, possibly leading to ADHD. (https://www.medicalnewstoday.com/articles/154164; Feb. 21, 2018)

The U.S. Public Health Service (USPHS) updated and replaced its 1962 Drinking Water Standards related to community water fluoridation to establish a single value of 0.7 mg/L as the optimal concentration of fluoride in drinking water. As established by the U.S. EPA, the maximum allowable level of naturally occurring fluoride in drinking water is 4 milligrams/liter (mg/L or ppm). Based on other toxicological reviews, the U.S. Environmental Protection Agency's (EPA) maximum contaminant level goal (MCLG) of 4 milligram per liter (mg/L or ppm) and secondary maximum contaminant level (SMCL) of 2 mg/L in drinking water. (Fluoridation Facts, A D A, 2018) Moreover, the majority of bottled waters on the market do not contain optimal levels (0.7 mg/L) of fluoride. Individuals who drink bottled water as their primary source of water could be missing the decay preventive effects of optimally fluoridated water available from their community water supplies. Tooth decay can be expected to increase if water fluoridation in a community is discontinued even if topical products such as fluoride toothpaste and fluoride mouth rinses are widely used.

It is readily apparent that in recent years the requirements for fluoride in water provided by inorganic chemicals such as sodium fluoride, sodium silicofluoride or hydrofluosilicic has been decreasing. Thus, there is a need for new dental compositions and/or food products that can provide additional sources of fluoride, preferably from natural organic compounds that do not have the drawbacks of currently available fluoride sources. There is also a need for compositions that can be administered via lollipops and lozenges that can stimulate salivary flow neutralize or increase the pH of the saliva and administer re-mineralization components so that a more effective remineralization can take place.

### SUMMARY

Compositions and methods are provided that effectively prevent, treat and reduce dental diseases such as carries in patients suffering from conditions that increase the acidity in the oral cavity.

In one embodiment, an oral composition is provided. The oral composition comprises crosslinked polyvinylpyrrolidone (PVP) xylitol and Match green tea and, in some aspects, the xylitol and crosslinked PVP are in a ratio of about 5:1. The oral composition is in the form of a lozenge or lollipop.

In one embodiment, an oral composition in the form of a lozenge or a lollipop for neutralizing saliva and re-hardening tooth enamel is provided. The oral composition comprises an alkalinizing agent, a re-mineralizing agent, a plasticizer, and a sugar alcohol. The base or lozenge core comprises crosslinked polyvinylpyrrolidone (PVP), the sugar alcohol comprises xylitol and the re-mineralizing agent is Matcha green tea.

In another embodiment, an oral composition for neutralizing saliva and re-hardening tooth enamel is provided, the oral composition comprising an alkalinizing agent; a re-mineralizing agent comprising Matcha green tea; a base; a plasticizer; and a sugar alcohol, wherein the oral composition is in the form of a lozenge or a lollipop.

In an exemplary embodiment, a multi-layer lozenge or lollipop for neutralizing saliva and re-hardening tooth enamel is provided. The lozenge or lollipop comprises an alkalinizing agent, a re-mineralizing agent which includes Matcha green tea, a base, a plasticizer, and a sugar alcohol. The lozenge or lollipop upon oral administration raises the saliva pH from about 7 to about 10 in about 1 minute. The multi-layer lozenge or lollipop further comprises an inner core and an outer layer, and upon oral administration the lollipop is dissolved within about 20 minutes. The base comprises crosslinked polyvinylpyrrolidone (PVP), the plasticizer comprises glycerin, and the sugar alcohol comprises xylitol.

In another embodiment, a method of making a multi-layer lozenge or lollipop for neutralizing saliva and re-hardening tooth enamel is provided. The method comprises: molding an inner core, the inner core comprising, xylitol, crosslinked polyvinylpyrrolidone (PVP), Matcha green tea and glycerin; and molding an outer layer over the inner core, the outer layer comprising xylitol, crosslinked PVP, glycerin, and L-arginine. Molding the inner core further comprises congealing the inner core in a mold for a period of time, and then inserting a stick into a portion of the inner core, and after the inserting of the stick into a portion of the inner core, the outer layer is then molded over the inner core.

In other embodiments, a method of treating, preventing or reducing a dental disease, for example carries is provided. The method comprises administering an oral composition in the form of a lozenge or lollipop, the composition comprising crosslinked polyvinylpyrrolidine (PVP), xylitol and Matcha green tea to treat, prevent or reduce the dental disease, for example carries. The method of treatment can also include administering the composition of this application to increase the pH of the saliva and/or enhance the hardening of enamel softened by acid.

Additional features and advantages of various embodiments will be set forth in part in the description that follows, and in part will be apparent from the description, or may be learned by practice of various embodiments. The objectives and other advantages of various embodiments will be realized and attained by means of the elements and combinations particularly pointed out in the description and appended claims.

### BRIEF DESCRIPTION OF THE DRAWINGS

In part, other aspects, features, benefits and advantages of the embodiments will be apparent with regard to the following description, appended claims and accompanying drawings where:

FIG. 1 is a top view of several multi-layer lollipops for neutralizing saliva and re-hardening tooth enamel;

FIG. 2 is a cross-sectional view a multi-layer lollipop of FIG. 1;

FIG. 3 is a graph of fluoride release from organic Matcha green tea from Navitas Organics;

FIG. 4 is a graph of fluoride release from non-organic Matcha green tea;

FIG. 5 is a graph of fluoride release from organic Matcha green tea of Usucha type;

FIG. 6 is a graph of a comparison among different types of Matcha green tea and their fluoride release;

FIG. 7 is a graph of a comparison among different types of Matcha green tea and their fluoride release;

FIG. 8 is a graph of calcium release from organic Matcha green tea;

FIG. 9 is a graph of calcium release from organic Matcha green tea;

FIG. 10 is a graph of calcium release from non-organic Matcha green tea;

FIG. 11 is a graph of a comparison among different types of Matcha green tea and their calcium release;

FIG. 12 is a graph of a comparison among different types of Matcha green tea and their calcium release;

FIG. 13 is a graph of fluoride release of organic Matcha green tea before and after grinding;

FIG. 14 is a graph of fluoride release of organic Matcha green tea before and after grinding;

FIG. 15 is a graph of a comparison of fluoride release of organic Matcha green tea before and after grinding;

FIG. 16 is a graph of calcium release of organic Matcha green tea before and after grinding;

FIG. 17 is a graph of calcium release of organic Matcha-Love green tea before and after grinding;

FIG. 18 is a graph of a comparison of calcium release of organic and non-organic Matcha green tea after grinding;

FIG. 19 is a graph of pH of organic Matcha green tea inner core;

FIG. 20 is a graph of fluoride release of organic Matcha green tea inner core;

FIG. 21 is a graph of calcium release of organic Matcha green tea inner core;

FIG. 22 is a graph of pH of the Double Layer Organic Matcha green tea lozenges;

FIG. 23 is a graph of pH of organic Matcha green tea inner core;

FIG. 24 is a graph of calcium release of organic Matcha green tea inner core having tripled calcium and phosphate content;

FIG. 25 is a graph of fluoride release of organic Matcha green tea inner core having tripled calcium and phosphate content;

FIG. 26 is a graph of fluoride free inner core where calcium and phosphate are tripled;

FIG. 27 is a graph of calcium release from organic Matcha green tea inner core having ten times calcium and phosphate content;

FIG. 28 is a graph of pH of the organic Matcha green tea inner core having ten times calcium and phosphate content;

FIG. 29 is a graph of fluoride release from the organic Matcha green tea inner core having ten times calcium and phosphate content;

FIG. 30 is a graph of fluoride release from the original inner core;

FIG. 31 is a graph of the calcium release from the original inner core;

FIG. 32 is a graph of pH of the original inner core;

FIG. 33 is a graph of fluoride release from the MI paste plus in deionized water;

FIG. 34 is a graph of calcium release from the MI paste plus in deionized water;

FIG. 35 is a graph of calcium release from the MI paste in deionized water;

FIG. 36 is a graph of fluoride release from the MI paste in deionized water;

FIG. 37 is a graph of calcium release from the MI paste in artificial saliva;

FIG. 38 is a graph of fluoride release from the MI paste in artificial saliva;

FIG. 39 is a graph of calcium release from the MI paste plus in artificial saliva;

FIG. 40 is a graph of fluoride release from the MI paste plus in artificial saliva; and

FIG. 41 is a graph of fluoride release from the three times fluoride-free inner cores.

It is to be understood that the figures are not drawn to scale. Further, the relation between objects in a figure may not be to scale, and may in fact have a reverse relationship as to size. The figures are intended to bring understanding and clarity to the structure of each object shown, and thus, some features may be exaggerated in order to illustrate a specific feature of a structure.

### DETAILED DESCRIPTION

For the purposes of this specification and appended claims, unless otherwise indicated, all numbers expressing quantities of ingredients, percentages or proportions of materials, reaction conditions, and other numerical values used in the specification and claims, are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques.

Notwithstanding that the numerical ranges and parameters setting forth, the broad scope of the invention are approximations, the numerical values set forth in the specific examples are reported as precisely as possible. Any numerical value, however, inherently contains certain errors necessarily resulting from the standard deviation found in their respective testing measurements. Moreover, all ranges disclosed herein are to be understood to encompass any and all subranges subsumed therein. For example, a range of “1 to 10” includes any and all subranges between (and including) the minimum value of 1 and the maximum value of 10, that is, any and all subranges having a minimum value of equal to or greater than 1 and a maximum value of equal to or less than 10, e.g., 5.5 to 10.

It is noted that, as used in this specification and the appended claims, the singular forms “a,” “an,” and “the,” include plural referents unless expressly and unequivocally limited to one referent. Thus, for example, reference to “an alkalinizing agent” includes one, two, three or more alkalinizing agents.

The term “lozenge,” as used herein, describes a solid or semi-solid substance that is dissolvable in the mouth. A lozenge can include, but is not limited to, a tablet, a troche, a cachou, pill, capsule, tab, a pellet, a dragee and/or a pastille.

The term “lollipop,” as used herein, describes a solid or semi-solid substance that is dissolvable in the mouth and is mounted on a stick.

Reference will now be made in detail to certain embodiments of the invention, examples of which are illustrated in the accompanying drawings. While the invention will be described in conjunction with the illustrated embodiments, it will be understood that they are not intended to limit the invention to those embodiments. On the contrary, the invention is intended to cover all alternatives, modifications, and equivalents, which may be included within the invention as defined by the appended claims.

The headings below are not meant to limit the disclosure in any way; embodiments under any one heading may be used in conjunction with embodiments under any other heading.

### Alkalinizing Agent

Alkalinizing agents are useful for neutralizing acids of the oral cavity. Alkalinizing agents can buffer the acids in the mouth and raise pH levels in acidic saliva from as low as 1.0 to 6.8 or greater. For example, treatment can be started when saliva pH is about 1.0, 1.5, 2.0, 2.5, 3.0, 3.5, 4.0, 4.5 or 5.0. The process of using an alkalinizing agent is useful for preventing and treating tooth erosion. Examples of alkalinizing agents include, but are not limited to, arginine, sodium carbonate, sodium bicarbonate, potassium carbonate, potassium bicarbonate, calcium carbonate, calcium bicarbonate, sesquicarbonates, borates, silicates, phosphates (e.g., monosodium phosphate, trisodium phosphate, pyrophosphate salts, etc.), imidazole or combinations thereof.

Saliva is a naturally occurring alkalinizing agent. Salivation is enhanced by agents stimulating the salivary glands. Saliva is the most significant natural defense in the oral cavity and is well known to possess several tooth-protective properties including anti-bacterial action, buffering capacity, cleansing effect, and re-mineralization activity.

In some embodiments, the alkalinizing agents may also comprise licorice root, eucalyptus or arginine from about 1%, 2%, 3%, 4%, 5%, 6%, 7% or to about 8%.

Alkalinizing agents may also comprise a re-hardening agent and buffering agent. Re-hardening of tooth enamel occurs when an agent capable of hardening tooth enamel is applied to the teeth after an acidic challenge to the teeth, which softens the enamel. Re-hardening agents are substances which are applied to the teeth to harden the softened enamel. Examples of re-hardening agents include, but are not limited to, Matcha green tea, calcium phosphate, fluoride, sodium fluoride, stannous fluoride, calcium chloride, potassium phosphate, casein phosphopeptide, caseinates, digests thereof or casein-derived phosphopeptides, amorphous calcium phosphate or combinations thereof. Enamel re-hardening is indicative of the effectiveness of various preventive treatments.

In some embodiments, the alkalinizing agent discussed above can be in the composition from about 0.01%, 0.05%, 0.10%, 0.15%, 0.20%, 0.25%, 0.30%, 0.35%, 0.40%, 0.45%, 0.50%, 0.55%, 0.60%, 0.65%, 0.70%, 0.75%, 0.80%, 0.85%, 0.90%, 0.95%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5% to about 10% % w/w, w/v, or v/v.

In some embodiments, the alkalinizing agent can raise the pH of saliva to above about 6.0, 6.5, 7.0, 7.5, 8.0, 8.5, 9.0, to about 9.5 or higher if desired.

In some embodiments, the rehardening agents can be in the composition from about 0.01%, 0.05%, 0.10%, 0.15%, 0.20%, 0.25%, 0.30%, 0.35%, 0.40%, 0.45%, 0.50%, 0.55%, 0.60%, 0.65%, 0.70%, 0.75%, 0.80%, 0.85%, 0.90%, 0.95%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5% to about 10% % w/w, w/v, or v/v.

In some embodiments, buffering agents include sodium chloride, 4-(2-hydroxyethyl)-1-piperazineethanesulfonic acid (HEPES), potassium hydroxide, potassium chloride, carboxylic, phosphoric and sulfonic acids, acid salts (e.g., monosodium citrate, disodium citrate, monosodium malate, etc.) or combinations thereof. Buffering agents can be added to the composition at concentrations of about 1 mM to about 300 mM. Buffering agents are added in sufficient quantity to adjust the pH of the composition from about 1.0 to 6.8, 6.9, 7.0 or greater.

### Remineralizing Agent

Re-mineralization is also useful for preventing and treating tooth erosion. Re-mineralization occurs when a mineral is added to the teeth to replace mineral components that have been depleted from the teeth. Softened enamel represents the stage of erosion where a remaining scaffold of mineral crystals can still be re-mineralized or re-hardened. Examples of re-mineralization agents include, fluoride, calcium, and/or phosphate. Fluoride enhances re-mineralization of early carious lesion by adsorbing onto the partially dissolved crystal lattice, which attracts calcium and phosphate ions to precipitate. In vitro and in situ studies have shown that a single or repetitive exposure to fluoride month rinse, fluoride gel, or 5000 ppm fluoride toothpaste, could slow down or prevent the erosive process. Fluoride, in the presence of calcium and phosphate, shifts the equilibrium surrounding the tooth surface towards re-mineralization.

More recently, federal agencies, such as the EPA, have been recommending increasing lower requirements for fluoride in drinking water. In some locals, the water does not have any added fluoride at all and supplementation with other natural organic sources could be used in order to avoid dental diseases caused by a lack of a fluoride source.

In various embodiments, Matcha green tea can be used as a source of fluoride. Both Matcha green tea and ordinary or regular supermarket type green tea are derived from the same plant called Camellia Sinensis. While green tea leaves usually come in the form of a tea bag, Matcha green tea is in powder form. Matcha green tea is actually 100% green tea leaves that have been ground into a fine powder. It is much more concentrated than regular green tea. With regular green tea, the leaves are brewed in hot water and then thrown out afterwards. By contrast, Matcha green tea drinkers consume the whole leaf dissolved in the water. Because of its concentrated form, Matcha green tea has up to 137 times the antioxidants and up to 10 times the nutritional content of regular green tea. Matcha green tea leaves are also grown under special shades to increase the chlorophyll and antioxidant levels even further. It is special in two aspects of farming and processing: the green tea plants used for Matcha green tea are shade-grown for three to four weeks before harvest, and the stems and veins are removed during processing. During shaded growth, the plant Camellia sinensis produces more theanine and caffeine.

The preparation of Matcha green tea leaves starts several weeks before harvest and may last up to 20 days, when the tea bushes are covered to prevent direct sunlight. This slows down growth, stimulates an increase in chlorophyll levels, turns the leaves a darker shade of green, and causes the production of amino acids, in particular theanine. Only the finest Matcha green tea buds are hand-picked. After harvesting, if the green tea leaves are rolled up before drying as in the production of a kind of green tea called sencha also known in Japan as jade dew tea, a tea which is prepared by infusing the processed whole tea leaves in hot water. This is as opposed to Matcha green tea, known as a powdered Japanese green tea, where the Matcha green tea powder is mixed with hot water and therefore the leaf itself is included in the beverage. If the leaves are laid out flat to dry, however, they will crumble somewhat and become known as tencha. Then, tencha may be deveined, destemmed, and stone-ground to the fine, bright green, talc-like powder known as Matcha green tea. Tencha is the name for tea leaves used for Matcha, before the leaves are ground into fine powder. Grinding the green tea leaves is a slow process because the mill stones must not get too warm, to avoid altering the aroma of the leaves. Up to one hour may be needed to grind 30 g of Matcha green tea.

The flavor of Matcha green tea is dominated by its amino acids. The high grade Match green teas contained lower amounts of total catechins than lower grade green teas (epigallocatechin (EGC) and epicatechin (EC) contents were greater in lower grade teas, while those of epigallocatechin gallate (EGCG) and epicatechin gallate (ECG) did not seem to correlate with tea grade), with the conclusion that the EGCG/EGC ratio reflected the quality of Matcha green tea more effectively than the EGC or total catechin contents. The relationship between the grade of Matcha green tea and caffeine contents seemed low. The chlorophyll contents were greater in the higher grade teas with a few exceptions, likely related to the strong shading used to cultivate high quality tencha. As used in this application, “green tea” or “extract of green tea” refers to green tea available at a supermarket which has not been grown and processed the same way Match green tea. “Matcha” or “Matcha green tea” refers to the Match green tea grown and processed as described above.

In some embodiments, the re-mineralizing agents can be added to the composition at about 0.01%, 0.05%, 0.10%, 0.15%, 0.20%, 0.25%, 0.30%, 0.35%, 0.40%, 0.45%, 0.50%, 0.55%, 0.60%, 0.65%, 0.70%, 0.75%, 0.80%, 0.85%, 0.90%, 0.95%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5% to about 10% % w/w, w/v, or v/v.

In some embodiments, the compositions of the present application further comprise an antimicrobial (e.g., antibacterial) agent. In some embodiments, one or more antimicrobial agents are optionally present. In some embodiments, one or more antimicrobial agents are optionally present in the amount of 0.05%, 0.10%, 0.15%, 0.20%, 0.25%, 0.30%, 0.35%, 0.40%, 0.45%, 0.50%, 0.55%, 0.60%, 0.65%, 0.70%, 0.75%, 0.80%, 0.85%, 0.90%, 0.95%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5% to about 10% w/w, w/v/, v/v.

In some embodiments, one or more antimicrobial agents, including but not limited to, ester parabens such as methyl paraben, ethyl paraben, propyl paraben, butyl paraben, benzyl paraben, or a combination thereof are optionally present in the amount of 0.10%, 0.15%, 0.20%, 0.25%, 0.30%, 0.35%, 0.40%, 0.45%, 0.50%, 0.55%, 0.60%, 0.65%, 0.70%, 0.75%, 0.80%, 0.85%, 0.90%, 0.95%, 1%, 1.5%, 2%, 2.5% to about 3% w/w, w/v/, v/v. Some embodiments of the present invention optionally comprise an antioxidant. Any orally acceptable antioxidant can be used, including butylated hydroxyanisole (BHA), butylated hydroxytoluene (BHT), vitamin A, carotenoids, vitamin E, flavonoids, polyphenols, ascorbic acid, herbal antioxidants, chlorophyll, melatonin, sodium sulfite, disodium metabisulfite, sodium bisulfite, and mixtures thereof in an amount of about 0.1%, 0.15%, 0.20%, 0.25%, 0.30%, 0.35%, 0.40%, 0.45%, 0.50%, 0.55%, 0.60%, 0.65%, 0.70%, 0.75%, 0.80%, 0.85%, 0.90%, 0.95%, 1%, 1.5%, 2%, 2.5% to about 3% w/w, w/v/, v/v.

In some embodiments, the compositions may comprise adhesion agents; viscosity modifiers; diluents; nonionic, cationic or amphoteric surfactants; foam modulators; humectants; mouth feel agents; sweeteners; flavoring agents; colorants, or combinations of two or more thereof.

The compositions of the present application are applied by the individual patient or the health care provider as an oral care composition, which in the ordinary course of usage, is not intentionally swallowed for purposes of systemic administration of particular therapeutic agents, but is rather retained in the oral cavity for a time sufficient to contact substantially all of the dental surfaces and/or oral tissues for purposes of oral activity (e.g., increase pH, harden enamel, remineralize teeth, etc.). The oral care composition may be in various forms including toothpaste, dentifrice, tooth gel, subgingival gel, mouth rinse or mouth wash, mousse, foam, mouth spray, lozenge, lollipop, chewable tablet, chewing gum or denture product. In one embodiment, the oral care composition is in a form selected from toothpaste, dentifrice, tooth gel, mouth wash or denture product. The oral care composition may also be incorporated onto strips or films for direct application or attachment to oral surfaces.

In one embodiment, the oral care composition comprises a mouthwash that is applied in two stages. First a therapeutically effective amount of an alkalinizing agent is applied to the teeth so as to raise saliva pH to 7.0 or above; and then a therapeutically effective amount of a re-mineralizing agent is applied to the teeth.

A “therapeutically effective amount” or “effective amount” is such that when administered, the alkalinizing and re-mineralizing results in alteration of the effects of acid in the oral cavity, such as, for example, prevention of tooth erosion or reduction of tooth erosion, re-hardening and re-mineralization of tooth enamel, etc.

The terms “treating” and “treatment” include “preventing” or “prevention” of disease or undesirable condition. In addition, “treating” or “treatment” does not require complete alleviation of signs or symptoms, does not require a cure, and specifically includes protocols that have only a marginal effect on the patient. “Reducing erosion” includes a decrease in erosion and does not require complete alleviation of erosion signs or symptoms, and does not require a cure.

In various embodiments, reducing erosion includes 5%, 10%, 15%, 20%, 25%, 30%, 35%, 40%, 45% or higher decrease in erosion.

In some embodiments, the alkalinizing agent can be applied to the oral cavity for a time of from about 5, 10, 15, 20, 25, 30, 35, 40, 45, 50, 55 to about 60 seconds. The alkalinizing agent can be applied to the oral cavity at a volume of about 5, 10, 15, 20, 25, 30, 35, 40, 45, 50, 55, 60, 65, 70, 75, 80, 85, 90, 95 to a volume of about 100 milliliters (mL).

In some embodiments, the re-mineralizing agent can be applied to the oral cavity for a time of about 5, 10, 15, 20, 25, 30, 35, 40, 45, 50, 55 to about 60 seconds. The re-mineralizing agent can be applied to the oral cavity at a volume of about 5, 10, 15, 20, 25, 30, 35, 40, 45, 50, 55, 60, 65, 70, 75, 80, 85, 90, 95 to a volume of about 100 mL.

In some embodiments, the alkalinizing agent is an alkalinizing mouthwash. The pH of the alkalinizing mouthwash may be 8.0 or greater.

In some embodiments, a kit is provided that may include additional parts along with the dental compositions. The kit may include a first mouthwash (e.g., alkalinizing agent) in a first compartment. The second compartment may include a second mouthwash (re-mineralizing agent) to be used in the oral cavity and other devices for administering it (e.g., calibrated cups, stirrers, etc.). A third compartment may include other procedural supplies, as well as an instruction booklets or links to websites for product information. A cover of the kit may include illustrations of using the dental composition and a clear plastic cover may be placed over the compartments to maintain sterility.

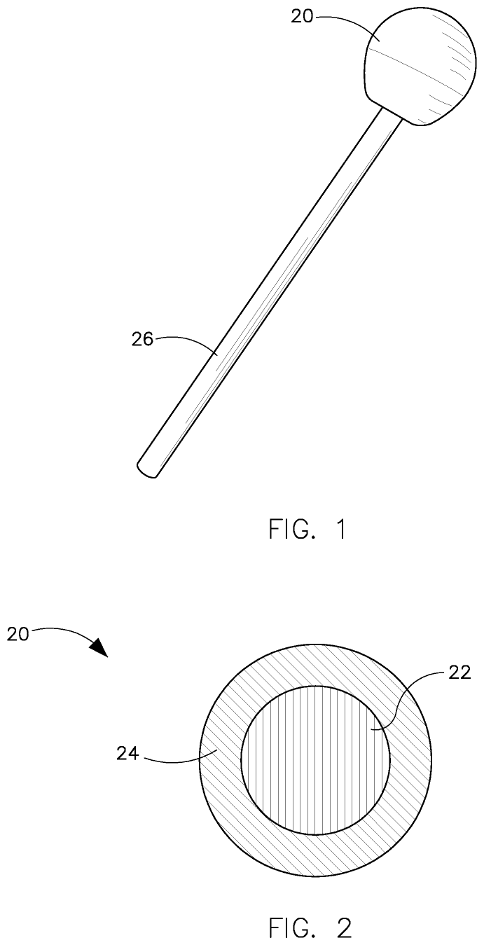

In one embodiment, an oral composition for neutralizing saliva and re-hardening tooth enamel is provided. The composition comprises an alkalinizing agent, a re-mineralizing agent, a base, a plasticizer, and a sugar alcohol. The composition is in the form of a lollipop 20 or a lozenge, as shown in FIGS. 1 and 2.

The base comprises polyvinylpyrrolidone (PVP) such as Kollidon® or crosslinked PVP such as Kollidon® Cl, the plasticizer comprises glycerin, and the sugar alcohol comprises xylitol. The base enhances stability and release of the components of the composition. The combination of Kollidon® and xylitol allows for a lollipop or lozenge that has a stable melting point since the low melting point of xylitol (e.g., about 92° C.) is not further lowered by the use of Kollidon® (e.g., melting point of about >140° C.).

The base, such as, for example, PVP or the crosslinked PVP, is in an amount of from about 5 to about 50% of the composition. The base such as, for example, the PVP or the crosslinked PVP, may be in an amount of about 5 to about 40%, 5 to about 30%, 5 to about 20%, 5 to about 10%, 10 to about 50%, 10 to about 40%, 10 to about 30%, 10 to about 20%, 20 to about 50%, 20 to about 40%, 20 to about 30%, 30 to about 50%, 30 to about 40%, or 40 to about 50% of the composition. In some embodiments, the base such as, for example, the PVP or the crosslinked PVP, is in an amount of from about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50% of the composition. In some embodiments, the base, such as, for example, the PVP or the crosslinked PVP, is in an amount of from about 1 to about 90% of the composition. In some embodiments, the base, such as, for example, the PVP or the crosslinked PVP is in an amount of about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, to about 90% of the composition.

The sugar alcohol, such as, for example, the xylitol, is in an amount of from about 20 to about 95% of the composition. In some embodiments, the sugar alcohol, such as for example, the xylitol, is in an amount of about 20 to about 85%, 20 to about 75%, 20 to about 65%, 20 to about 55%, 20 to about 45%, 20 to about 35%, 30 to about 95%, 30 to about 85%, 30 to about 75%, 30 to about 65%, 30 to about 55%, 30 to about 45%, 40 to about 95%, 40 to about 85%, 40 to about 75%, 40 to about 65%, 40 to about 55%, 50 to about 95%, 50 to about 85%, 50 to about 75%, 50 to about 65%, 60 to about 95%, 60 to about 85%, 60 to about 75%, 70 to about 95%, 70 to about 85%, or 80 to about 95% of the composition. In some embodiments, the sugar alcohol, such as, for example, the xylitol, is in an amount of from about 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, or 95% of the composition.

The plasticizer, such as, for example, the glycerin, is in an amount of from about 0.1 to about 1% of the composition. In some embodiments, the plasticizer, such as, for example, the glycerin, is in an amount of about 0.1 to about 0.8%, 0.1 to about 0.6%, 0.1 to about 0.4%, 0.1 to about 0.2%, 0.3 to about 1%, 0.3 to about 0.8%, 0.3 to about 0.6%, 0.3 to about 0.4%, 0.5 to about 1%, 0.5 to about 0.8%, 0.5 to about 0.6%, 0.7 to about 1%, or 0.7 to about 0.8% of the composition. In some embodiments, the plasticizer, such as, for example, the glycerin is in an amount of from about 0.1, 0.2, 0.3, 0.4, 0.5, 0.6, 0.7, 0.8, 0.9, to about 1% of the composition. In some embodiments, the plasticizer, such as, for example, the glycerin, is in an amount of from about 0.1 to about 5% of the composition. In some embodiments, the plasticizer, such as, for example, the glycerin is in an amount of from about 0.1, 0.2, 0.3, 0.4, 0.5, 0.6, 0.7, 0.8, 0.9, 1, 2, 3, 4, to about 5% of the composition.

In some embodiments, the base and the sugar alcohol are in a particular ratio in the composition. For example, the xylitol and crosslinked PVP are in a 5:1 ratio. The crosslinked PVP can be Kollidon® Cl, and the xylitol and crosslinked PVP are in a 5:1 ratio. In some embodiments, the ratio of xylitol and crosslinked PVP can be from 10:1, 9:1, 8:1, 7:1, 6:1, 5:1, 4:1, 3:1 to about 2:1. In some embodiments, the oral composition can comprise only a sugar alcohol and a base in a 5:1 ratio.

In some embodiments, the base, can alternatively or in addition to the crosslinked PVP be hydroxypropyl methylcellulose (HPMC), methylcellulose, hydrophilic polymers for controlled release, and/or hydrophobic polymers. In some embodiments, the hydrophilic polymers, include, but are not limited to a polyacrylate, an alginate, chitosan, a hydrophilic polyamine, a chitosan derivative, polylysine, polyethylene, xanthan, carrageenan, chondroitin sulfate, a starch, a modified cellulosic polymer, a dextran, and/or hyaluronan. In some embodiments, the hydrophobic polymers include, but are not limited to, phthalic acid esters, polyvinyl acetate phthalate, cellulose acetate phthalate, methacrylic acid esters, cellulose ethers, polyethylene oxide polymers, and/or ethylcellulose.

In some embodiments, the plasticizer, can alternatively or in addition to the glycerin be polyethylene glycol (PEG), PEG 200, PEG 300, PEG 400, propylene glycol and/or citrate esters.

In some embodiments, the sugar alcohol can alternatively or in addition to the xylitol be sorbitol and/or mannitol.

The alkalinizing agent can include the alkalinizing agents described above and in the amounts disclosed. For example, the alkalinizing agent can comprise a re-hardening agent, eucalyptus oil, licorice root, and/or arginine, such as L-arginine.

The eucalyptus oil can be in an amount of from about 0.01 to about 0.5% of the composition. In some embodiments, the eucalyptus oil is in an amount of from about 0.01, 0.02, 0.03, 0.04, to about 0.5% of the composition. In some embodiments, the eucalyptus oil can be in an amount of from about 0.01 to about 3% of the composition. In some embodiments, the eucalyptus oil is in an amount of from about 0.01, 0.02, 0.03, 0.04, 0.5, 0.6, 0.7, 0.8, 0.9, 1, 2, to about 3% of the composition.

The licorice root can be in an amount of from about 0.01 to about 1% of the composition. In some embodiments, the licorice root can be in an amount of from about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, to about 1% of the composition. In some embodiments, the licorice root can be in an amount of from about 0.01 to about 3% of the composition. In some embodiments, the licorice root can be in an amount of from about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, to about 3% of the composition.

The arginine, such as L-arginine, can be in an amount of from about 1 to about 8% of the composition. In some embodiments, the arginine, such as L-arginine can be in an amount of from about 1, 2, 3, 4, 5, 6, 7, to about 8% of the composition. The arginine, such as L-arginine, can be in an amount of from about 1 to about 20% of the composition. In some embodiments, the arginine, such as L-arginine, can be in an amount of from about 1, 2, 3, 4, 5, 6, 7, 8, 9, 10, 11, 12, 13, 14, 15, 16, 17, 18, 19, to about 20% of the composition.

The alkalinizing agent can also include sodium bicarbonate, or potassium bicarbonate, and the re-hardening agent can include calcium chloride, potassium phosphate and/or casein phosphopeptide.

The alkalinizing agent, such as, for example, the casein phosphopeptide, can be in an amount of from about 1 to about 5% of the composition. In some embodiments, the alkalinizing agent, such as, for example, the casein phosphopeptide, can be in an amount of from about 1, 2, 3, 4, to about 5% of the composition. In some embodiments, the alkalinizing agent, such as, for example, the casein phosphopeptide, can be in an amount of from about 1 to about 20% of the composition. In some embodiments, the alkalinizing agent, such as, for example, the casein phosphopeptide, can be in an amount of from about 1, 2, 3, 4, 5, 6, 7, 8, 9, 10, 11, 12, 13, 14, 15, 16, 17, 18, 19, to about 20% of the composition.

The re-mineralizing agent can include the re-mineralizing agents described above and the amounts disclosed. For example, the re-mineralizing agent can include calcium chloride, monopotassium phosphate, and/or sodium fluoride.

The re-mineralizing agent, such as, for example, the calcium chloride, can be in an amount of from about 0.01 to about 0.5% of the composition. In some embodiments, the re-mineralizing agent, such as, for example, the calcium chloride, can be in an amount of from about 0.01 to about 0.2%, from about 0.01 to about 0.08%, from about 0.01 to about 0.04%, from about 0.03 to about 0.5%, from about 0.03 to about 0.5%, from about 0.03 to about 0.2%, from about 0.03 to about 0.08%, from about 0.03 to about 0.04%, from about 0.05 to about 0.5%, from about 0.05 to about 0.2%, from about 0.05 to about 0.08%, from about 0.07 to about 0.5%, from about 0.07 to about 0.2%, or from about 0.07 to about 0.8%. In some embodiments, the re-mineralizing agent, such as, for example, the calcium chloride, can be in an amount of from about 0.01, 0.02, 0.03, 0.04, 0.05, 0.06, 0.07, 0.08, 0.09, 0.1, 0.2, 0.3, 0.4 or 0.5% of the composition. In some embodiments, the re-mineralizing agent, such as, for example, the calcium chloride, can be in an amount of from about 0.01 to about 15% of the composition. In some embodiments, the re-mineralizing agent, such as, for example, the calcium chloride, can be in an amount of from about 0.01, 0.02, 0.03, 0.04, 0.05, 0.06, 0.07, 0.08, 0.09, 0.1, 0.2, 0.3, 0.4 or 0.5, 0.6, 0.7, 0.8, 0.9, 1, 2, 3, 4, 5, 6, 7, 8, 9, 10, 11, 12, 13, 14, to about 15% of the composition.

The re-mineralizing agent, such as, for example, the monopotassium phosphate can be in an amount of from about 0.01 to about 0.5% of the composition. In some embodiments, the re-mineralizing agent, such as, for example, the monopotassium phosphate, can be in an amount of from about 0.01 to about 0.2%, from about 0.01 to about 0.08%, from about 0.01 to about 0.04%, from about 0.03 to about 0.5%, from about 0.03 to about 0.5%, from about 0.03 to about 0.2%, from about 0.03 to about 0.08%, from about 0.03 to about 0.04%, from about 0.05 to about 0.5%, from about 0.05 to about 0.2%, from about 0.05 to about 0.08%, from about 0.07 to about 0.5%, from about 0.07 to about 0.2%, or from about 0.07 to about 0.8%. In some embodiments, the re-mineralizing agent, such as, for example, the monopotassium phosphate, can be in an amount of from about 0.01, 0.02, 0.03, 0.04, 0.05, 0.06, 0.07, 0.08, 0.09, 0.1, 0.2, 0.3, 0.4 or 0.5% of the composition. In some embodiments, the re-mineralizing agent, such as, for example, the monopotassium phosphate, can be in an amount of from about 0.01 to about 15% of the composition. In some embodiments, the re-mineralizing agent, such as, for example, the monopotassium phosphate, can be in an amount of from about 0.01, 0.02, 0.03, 0.04, 0.05, 0.06, 0.07, 0.08, 0.09, 0.1, 0.2, 0.3, 0.4 or 0.5, 0.6, 0.7, 0.8, 0.9, 1, 2, 3, 4, 5, 6, 7, 8, 9, 10, 11, 12, 13, 14, to about 15% of the composition.

The re-mineralizing agent, such as, for example, the sodium fluoride, can be in an amount of from about 0.01 to about 1% or from about 0.01 to about 0.1% of the composition. In some embodiments, the re-mineralizing agent, such as, for example, the sodium fluoride, can be in an amount of from about 0.01 to about 0.5%, from about 0.01 to about 0.08%, from about 0.01 to about 0.04%, from about 0.03 to about 0.1%, from about 0.03 to about 0.5%, from about 0.03 to about 0.2%, from about 0.03 to about 0.08%, from about 0.03 to about 0.04%, from about 0.05 to about 1%, from about 0.05 to about 0.5%, from about 0.05 to about 0.2%, from about 0.05 to about 0.08%, from about 0.07 to about 1%, from about 0.07% to about 0.5%, from about 0.07 to about 0.2%, or from about 0.07 to about 0.8%. In some embodiments, the re-mineralizing agent, such as, for example, the sodium fluoride, can be in an amount of from about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, or 1% of the composition. In some embodiments, the re-mineralizing agent, such as, for example, the sodium fluoride, can be in an amount of from about 0.01 to about 15% of the composition. In some embodiments, the re-mineralizing agent, such as, for example, the sodium fluoride, can be in an amount of from about 0.01, 0.02, 0.03, 0.04, 0.05, 0.06, 0.07, 0.08, 0.09, 0.1, 0.2, 0.3, 0.4 or 0.5, 0.6, 0.7, 0.8, 0.9, 1, 2, 3, 4, 5, 6, 7, 8, 9, 10, 11, 12, 13, 14, to about 15% of the composition.

The composition is administered to a user's teeth in the form of a lollipop or lozenge for a time of from about 1 to about 20 minutes. The composition is administered to a user's teeth fora period of time from about 1, 2, 3, 4, 5, 6, 7, 8, 9, 10, 11, 12, 13, 14, 15, 16, 17, 18, 19, to about 20 minutes.

The composition raises the user's saliva pH to about 7 or greater in about 1 minute. In some embodiments, the composition raises the user's saliva pH to about 7, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, or 10.

The composition can also include green tea extract in an amount of from about 0.01 to about 1%. In some embodiments, the green tea extract is in an amount of from about 0.01, 0.02, 0.03, 0.04, 0.05, 0.06, 0.07, 0.08, 0.09, to about 1% of the composition. In some embodiments, the composition can also include green tea extract in an amount of from about 0.01 to about 5%. In some embodiments, the green tea extract is in an amount of from about 0.01, 0.02, 0.03, 0.04, 0.05, 0.06, 0.07, 0.08, 0.09, 1, 2, 3, 4, to about 5% of the composition.

As described above, the composition is in the form of a lollipop of lozenge. In some embodiments, a multi-layer lollipop 20 is provided that is formed from the composition. The lollipop comprises an inner core 22, an outer layer 24 and a stick 26. The lollipop is configured to be inserted into a patient's mouth to stimulate salivary flow of a patient, neutralize the patient's saliva, and as the patient sucks and continues to suck on the lollipop, the lollipop releases components of the composition to assist in replenishing softened enamel in the teeth. The lollipop is able to stay in the mouth of a patient for a longer period of time than toothpastes and/or mouthwashes, thereby increasing the effectiveness of teeth re-mineralization. In some embodiments, upon oral administration, the lollipop dissolves in about 20 minutes.

The lollipop can be formed from the composition via molding methods described herein. Since the lollipop is formed from the composition, the amount of each component in the lollipop will be the same as in the composition, as described above. In some embodiments, the inner core of the lollipop is formed before the outer layer, and comprises components of the composition, such as, a sugar alcohol, a base, one or more alkalinizing agents, one or more re-mineralizing agents, a plasticizer, and an extract such as green tea extract.

In one embodiment, the inner core of the lollipop comprises xylitol, crosslinked PVP, such as Kollidon® Cl, potassium monophosphate, calcium chloride, Matcha green tea, glycerin, green tea extract, licorice extract, eucalyptus oil, and casein phosphopeptide.

In some embodiments, the outer layer of the lollipop is formed after the inner core, and is then molded over the inner core. The outer layer comprises components of the composition, such as, a sugar alcohol, a base, a plasticizer, one or more alkalinizing agents, and an extract such as green tea extract.

In other embodiments, the outer layer comprises other natural ingredients that can be added as flavorants and/or for their acid reducing and antibacterial properties. Examples of useful natural ingredients that can be included in the outer layer of the lozenge or lollipop include without limitation ginger, peppermint, chamomile, licorice, coconut oil. Ginger can be available in various forms, such as dried powder, crystallized, in capsules, and as an extract. In many consumer products, for example, in ginger teas, the content of ginger is in an amount from about 0.25 g to about 2 g of ginger. In some teas, for example a bag of Yogi tea, the amount of ginger is about 1.5 g per tea bag.

Peppermint is another flavorant that can be added in the outer layer of the lozenges or lollipops described in this application. Peppermint is available as an essential oil or in tea leaves. Peppermint can have a neutralizing effect in an acid environment and can be present in an amount from about 0.25 to about 2 grams per composition.

Other flavorants include chamomile and coconut oil. There is no standard dose for chamomile use. Studies have used between 220 milligrams to 1,600 milligrams daily in capsule form. Chamomile is frequently consumed as tea. Coconut oil has an antibacterial effect and can be added in small amounts.

In one embodiment, the outer layer comprises xylitol, crosslinked PVP, such as Kollidon® Cl, glycerin, green tea extract, licorice extract, eucalyptus oil and L-arginine.

In some embodiments, a multi-layered lollipop or lozenge is provided that comprises crosslinked PVP and xylitol, or crosslinked PVP, xylitol and glycerin where the ratio of xylitol to crosslinked PVP is 5:1. In this embodiment, any of the components described above may be added to the lollipop or lozenge, as well as any other active pharmaceutical ingredients contemplated.

In some embodiments, the multi-layered lollipop can have more than 2 layers. In some embodiments, the multi-layered lollipop can have 2, 3, 4, 5, 6, 7, 8, 9, or 10 layers depending on the release desired. In one embodiment, the lollipop can be a single monolithic lollipop with no layers.

The lollipop as described above, is administered to a user's teeth for a period of time from about 1 to about 20 minutes. The lollipop is administered to a user's teeth for a time of from about 1, 2, 3, 4, 5, 6, 7, 8, 9, 10, 11, 12, 13, 14, 15, 16, 17, 18, 19, to about 20 minutes. The re-mineralizing agents, such as, for example, the calcium chloride and the monopotassium phosphate contained in the lollipop will release during the period time of from about 1 to about 20 minutes to assist in the replenishing of softened tooth enamel, and the sodium fluoride will assist in remineralization of tooth structures.

In some embodiments, the outer layer, the inner layer and/or the stick of the lollipop can be naturally dyed to have various colors such as pink, peach, red, white, yellow, orange, purple, green and/or blue. In some embodiments, the outer layer and the inner layer can be naturally dyed in the same or different colors. In some embodiments, the dyes are made so as not to stain the patient's teeth.

In some embodiments, the components of the lollipop can be released from the lollipop via controlled and/or immediate release over the period of time of from about 1 to about 20 minutes, described herein. In some embodiments, the components of the lollipop may be released via microspheres.

In some embodiments, a lollipop or lozenge is provided that is single layered. The single layered lozenge or lollipop comprises a neutralizing basic salt, such as, for example, arginine on an outer layer that initially dissolves with a patient's saliva, neutralizing the acidity caused by consumption of a low pH food or acidic regurgitation. The single layered lollipop or lozenge also includes a core comprising calcium chloride, potassium monophosphate, Matcha green tea, casein phosphopeptide, green tea extract, xylitol, licorice extract and eucalyptus oil.

In some embodiments, a lollipop or lozenge is provided that is multi-layered. In some embodiments, the liquid mouthwash described herein is made into a solid lollipop or lozenge.

In some embodiments, a mold is used to form the lollipop or lozenge, and the lollipop or lozenge can weigh up to 12 g, and in some cases 5 g. The multi-layered lollipop or lozenge comprises a neutralizing basic salt in the outer layer, such as, for example, arginine, and licorice extract; a second neutralizing layer comprising a protein, such as, for example, casein, green tea extract, xylitol, and a base comprising PEG 1440; and a soft gelatin core of calcium chloride, monopotassium phosphate, sodium fluoride and PEG 1440. The neutralizing basic salt on the outer layer initially dissolves with the saliva, neutralizing the acidity caused by consumption of a low pH food or acidic regurgitation. The protein (e.g., casein) in the second layer continues with neutralization and also provides calcium. The softer consistency of the gelatin core comprising the calcium chloride, monopotassium phosphate, sodium fluoride replenishes the mineral deficit in the teeth and also helps with the remineralization process. Table 1 below summarizes the components and the particular amounts used in the single layered and multi-layered lollipops and lozenges.

TABLE 1
Components of the single layer and
Multi-layer Lollipop and Lozenge

| 1. | Outer core: | Amount | |
|---|---|---|---|
| 2. | Xylitol | 5.71 | g |
| 3. | Kollidon Cl | 1.14 | g |
| 4. | Glycerin | 0.07 | g |
| 5. | Green tea extract | 0.0025 | g |
| 6. | Licorice extract | 0.0025 | g |
| 7. | Eucalyptus oil | 0.0025 | g |
| 8. | L-Arginine | 0.07 | g |
| | | Total 7.0 g | |
| 9. | Inner core: | | |
| 10. | Casein phosphopeptide | 0.1 | g |
| 11. | Calcium Chloride | 0.0022 | g |
| 12. | Potassium monophosphate | 0.0010 | g |
| 13. | Matcha green tea | 0.513 | g |
| 14. | Xylitol | 3.528 | g |
| 15. | Kollidon Cl | 0.80 | g |
| 16. | Glycerin | 0.05 | g |
| 17. | Green tea extract | 0.0017 | g |
| 18. | Licorice extract | 0.0017 | g |
| 19. | Eucalyptus oil | 0.0017 | g |

Total 5.0 g

### Method of Making

In some embodiments, a method of making a multi-layer lollipop for neutralizing saliva and re-hardening tooth enamel is provided. The method comprises: molding an inner core, the inner core comprising, xylitol, crosslinked polyvinylpyrrolidone (PVP), and glycerin; and molding an outer layer over the inner core, the outer layer comprising xylitol, crosslinked PVP, glycerin, and L-arginine. In some embodiments, the inner core further comprises, potassium monophosphate, calcium chloride, Matcha green tea, green tea extract, licorice extract, eucalyptus oil, and casein phosphopeptide; and the outer layer further comprises green tea extract, licorice extract, and eucalyptus oil.

In some embodiments, molding the inner core further comprises congealing the inner core in a mold for a period of time, and then inserting a stick into a portion of the inner core, and after the inserting of the stick into a portion of the inner core, the outer layer is then molded over the inner core.

In some embodiments, the Matcha containing core of the lozenge or lollipop is prepared by placing xylitol in a 200 ml glass beaker and adjust heat at 150° C. until the xylitol is completely melted and turned into a liquid. Kollidon CL is added and melted into the liquid xylitol until a homogeneous mixture is obtained. A few drops of glycerin and the remaining components of licorice extract, green tea extract, KH₂PO₄, Matcha, eucalyptus oil, casein phosphopeptide, mango flavor, CaCl₂ are added with mixing and heat to ensure a homogeneous mixture. The inner core mixture is then poured into a small mold that has been previously lubricated with canola or vegetable oil. The inner core is then allowed to congeal and then it is removed from the mold and immersed into the outer layer and allowed to congeal again. The outer coating layer is prepared by adding an amount of xylitol to a glass container, heating the xylitol to a temperature of 150° C. until the xylitol becomes clear, stirring the xylitol until it is melted, adding an amount of crosslinked PVP to the glass container to form a mixture, adding an amount of glycerin to the mixture, sequentially adding an amount of green tea extract, an amount of licorice extract and then an amount eucalyptus oil to the mixture, quickly adding and mixing L-arginine to the mixture, pouring the mixture into the mold immediately after the L-arginine is mixed into the mixture, and congealing the mixture at room temperature for a period time of from about 24 to about 48 hours to form the multi-layered lollipop.

Conditions Associated with Dental Erosion

Patients with certain chronic health conditions have been shown to have significantly higher erosive tooth wear than a control group.¹¹ Erosive tooth wear from hydrochloric acid when stomach juice is involuntarily regurgitated has been associated with chronic health issues such as in GERD, hiatal hernia, or occur through chronic vomiting like in bulimia nervosa (Schroeder et al., 1995; Valena and Young, 2002; Barron et al., 2003). Prevalence of dental erosion in GERD patients was 24% (Pace et al., 2008). Preventive treatments that can reduce tooth damage from hydrochloric acid erosion will certainly benefit patients suffering from stomach acid regurgitation due to GERD and eating disorders. According to the National Eating Disorders Association, approximately 30 million Americans suffer from eating disorders. The acts of alkalinizing, re-hardening and re-mineralizing are important in treating and preventing dental erosion in patients suffering from these diseases and in need of such treatment. An acidic challenge in a patient suffering from GERD or bulimia, can cause an under saturation of salivary salts (calcium, phosphate), which can contribute to the demineralization of tooth structure.^{9,10} Delivery of these minerals to the oral environment by self-application through a mouth rinse or tooth cream is a practical approach for patients with acid regurgitation.

### EXAMPLES

Studies were conducted to examine the release of fluoride and calcium from different types of Matcha green tea. Some Matcha green teas were organic and some were not organic. In some examples, the release of fluoride and calcium was measured before and after grinding of the Matcha green tea. Further studies were conducted to measure properties of the inner core of the lozenge of this application, for example the pH, fluoride and calcium release of the inner core of organic Matcha green tea. Studies of the hardness of organic Matcha green tea were also conducted.

### Example 1: Fluoride Release of Organic Matcha Green Tea

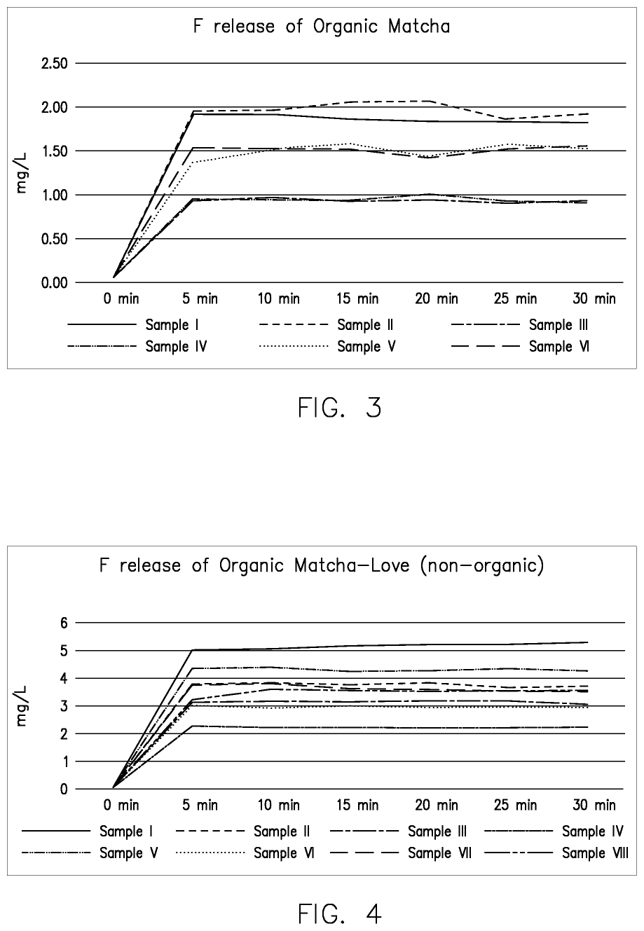

In this example fluoride release from organic Matcha green tea was investigated. Six samples of organic Matcha powder tea available from Navitas Organics were dissolved in deionized water (DIW) according to the conditions described in the Table 2 below. The fluoride release was measured at 5 minutes intervals for 30 minutes as summarized in Table 3 below as also illustrated in FIG. 3.

TABLE 2

| | | | |
|---|---|---|---|
| Sample I: | 1 gm Matcha powder | Speed 250 RPM | At room temp. |
| | in 30 ml of DIW | | |
| Sample II: | 1 gm Matcha powder | Speed 250 RPM | At 37° C. |
| | in 30 ml of DIW | | |
| Sample III: | 1 gm Matcha powder | Speed 400 RPM | At room temp. |
| | in 60 ml of DIW | | |
| Sample IV: | 1 gm Matcha powder | Speed 250 RPM | At room temp. |
| | in 60 ml of DIW | | |
| Sample V: | 2 gm Matcha powder | Speed 250 RPM | At room temp. |
| | in 60 ml of DIW | | |
| Sample VI: | 2 gm Matcha powder | Speed 400 RPM | At room temp. |
| | in 60 ml of DIW | | |

TABLE 3

| | Sample | Sample | Sample | Sample | Sample | Sample |
|---|---|---|---|---|---|---|
| Time | I | II | III | IV | V | VI |
| 0 min | 0.05 | 0.07 | 0.06 | 0.07 | 0.08 | 0.05 |
| 5 min | 1.91 | 1.95 | 0.94 | 0.95 | 1.37 | 1.54 |
| 10 min | 1.92 | 1.96 | 0.97 | 0.94 | 1.52 | 1.53 |
| 15 min | 1.86 | 2.06 | 0.94 | 0.94 | 1.57 | 1.52 |
| 20 min | 1.83 | 2.07 | 0.94 | 1.01 | 1.42 | 1.42 |
| 25 min | 1.83 | 1.86 | 0.91 | 0.92 | 1.57 | 1.52 |
| 30 min | 1.82 | 1.92 | 0.93 | 0.91 | 1.54 | 1.56 |

### Example 2: Fluoride Release from Non-Organic Matcha Green Tea

This study investigated fluoride release from non-organic Matcha green tea. Eight samples of non-organic Matcha Love stone ground powder tea available from Usucha were prepared according to the conditions described in the Table 4 below. The fluoride release was measured at 5 minutes intervals for 30 minutes as summarized in Table 5 below as also illustrated in FIG. 4.

TABLE 4
All tests had been done at Room Temp.

Sample I: 2 g Matcha-Love powder in 60 ml DIW, at 250 RPM.
Sample II: 2 g Matcha-Love powder in 60 ml DIW, at 400 RPM.
Sample III: 1 g Matcha-Love powder in 60 ml DIW, at 250 RPM.
Sample IV: 1 g Matcha-Love powder in 60 ml DIW, at 400 RPM.
Sample V: 1.5 g Matcha-Love powder in 60 ml DIW, at 250 RPM.
Sample VI: 1.5 g Matcha-Love powder in 60 ml DIW, at 400 RPM.
Sample VII: 1 g Matcha-Love powder in 30 ml DIW, at 250 RPM.
Sample VIII: Repeat the fluoride release test for Sample I.

TABLE 5

| Time | Sample I | Sample II | Sample III | Sample IV | Sample V | Sample VI | Sample VII | Sample VIII |
|---|---|---|---|---|---|---|---|---|
| 0 min | 0.06 | 0.06 | 0.05 | 0.08 | 0.06 | 0.05 | 0.06 | 0.06 |
| 5 min | 5.02 | 3.79 | 3.16 | 2029 | 4.39 | 3.06 | 3.77 | 3.25 |
| 10 min | 5.07 | 3.82 | 3.17 | 2.21 | 4.40 | 2.93 | 3.83 | 3.61 |
| 15 min | 5.19 | 3.77 | 3.16 | 2.21 | 4.26 | 2.99 | 3.64 | 3.56 |
| 20 min | 5.22 | 3.85 | 3.19 | 2.21 | 4.27 | 2.96 | 3.58 | 3.53 |
| 25 min | 5.23 | 3.68 | 3.17 | 2.22 | 4.37 | 2.98 | 3.51 | 3.57 |
| 30 min | 5.31 | 3.73 | 3.09 | 2.23 | 4.29 | 2.96 | 3.51 | 3.55 |

### Example 3: Fluoride Release from Organic Matcha Green Tea

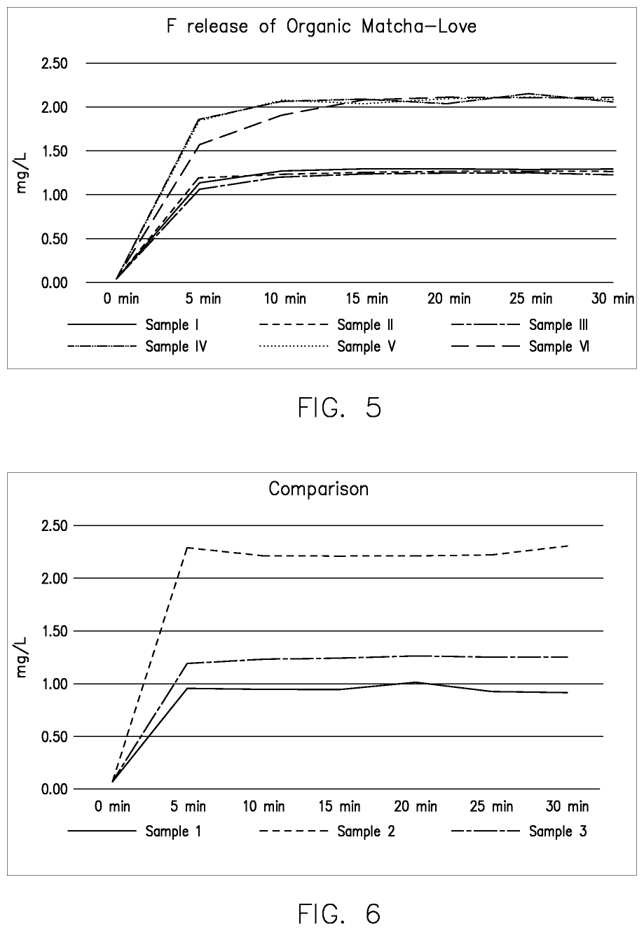

In this example fluoride release from organic Matcha green tea was investigated. Six samples of organic Matcha Love stone ground powder tea available from Usucha were prepared according to the conditions described in the Table 6 below. The fluoride release was measured at 5 minutes intervals for 30 minutes as summarized in Table 7 below as also illustrated in FIG. 5.

TABLE 6
Sample I: 1 g Matcha-Love Organic powder in 60 ml DIW, 150 RPM at
room temp.
Sample II: 1 g Matcha-Love Organic powder in 60 ml DIW, 250 RPM at
room temp.
Sample III: 1 g Matcha-Love Organic powder in 60 ml DIW, 400 RPM at
room temp.
Sample IV: 2 g Matcha-Love Organic powder in 60 ml DIW, 150 RPM at
room temp.
Sample V: 2 g Matcha-Love Organic powder in 60 ml DIW, 250 RPM at
room temp.
Sample VI: 2 g Matcha-Love Organic powder in 60 ml DIW, 400 RPM at
room temp.

TABLE 7

| | Sample | Sample | Sample | Sample | Sample | Sample |
|---|---|---|---|---|---|---|
| Time | I | II | III | IV | V | VI |
| 0 min | 0.04 | 0.06 | 0.04 | 0.04 | 0.05 | 0.07 |
| 5 min | 1.13 | 1.19 | 1.06 | 1.86 | 1.85 | 1.57 |
| 10 min | 1.27 | 1.23 | 1.20 | 2.07 | 2.08 | 1.91 |
| 15 min | 1.30 | 1.24 | 1.23 | 2.07 | 2.04 | 2.09 |
| 20 min | 1.30 | 1.26 | 1.25 | 2.04 | 2.10 | 2.12 |
| 25 min | 1.29 | 1.25 | 1.25 | 2.15 | 2.11 | 2.10 |
| 30 min | 1.30 | 1.25 | 1.23 | 2.07 | 2.10 | 2.11 |

### Example 4: Comparison Among Different Types of Matcha Green Tea and their Fluoride Release

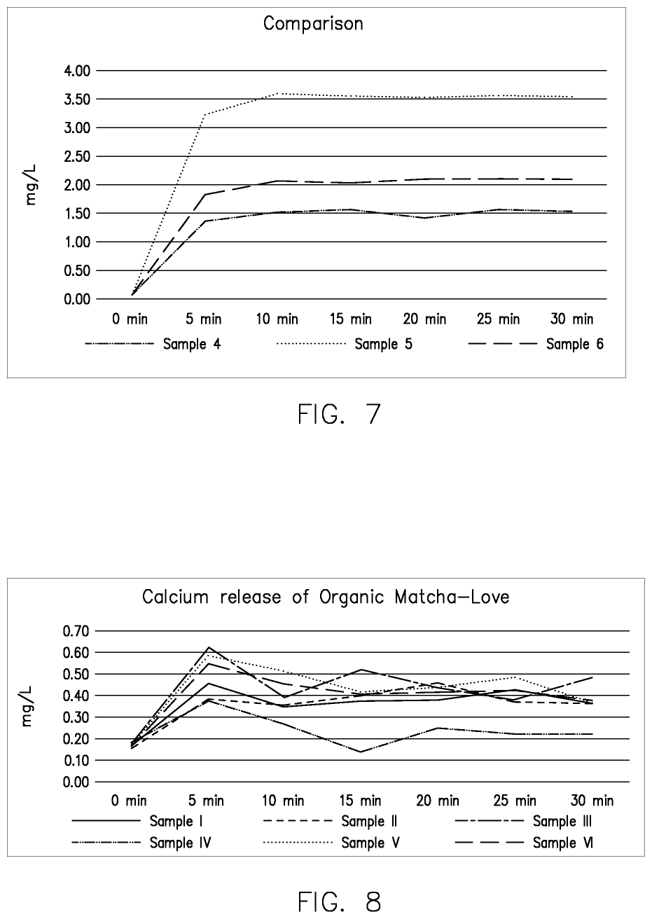

In this example different types of Matcha green tea and their fluoride release were compared. The samples were prepared according to the conditions summarized in Tables 8 and 10. The comparison results are outlined in Tables 9 and 11 and illustrated in FIGS. 6 and 7.

TABLE 8
Sample I: 1 g Organic Matcha in 60 ml DIW, 250 RPM at room temp.
Sample II: 1 g Matcha-Love in 60 ml DIW, 250 RPM at room temp.
Sample III: 1 g Organic Matcha-Love in 60 ml DIW, 250 RPM at room
temp.

TABLE 9

| Time | Sample 1 | Sample 2 | Sample 3 |
|---|---|---|---|
| 0 min | 0.07 | 0.08 | 0.06 |
| 5 min | 0.95 | 2.29 | 1.19 |
| 10 min | 0.94 | 2.21 | 1.23 |
| 15 min | 0.94 | 2.21 | 1.24 |
| 20 min | 1.01 | 2.21 | 1.26 |
| 25 min | 0.92 | 2.22 | 1.25 |
| 30 min | 0.91 | 2.30 | 1.25 |

TABLE 10
Sample 4: 2 g Organic Matcha in 60 ml DIW, 250 RPM at room temp.
Sample 5: 2 g Matcha-Love in 60 ml DIW, 250 RPM at room temp.
Sample 6: 2 g Organic Matcha-Love in 60 ml DIW, 250 RPM at room
temp.

TABLE 11

| Time | Sample 4 | Sample 5 | Sample 6 |
|---|---|---|---|
| 0 min | 0.08 | 0.06 | 0.05 |
| 5 min | 1.37 | 3.25 | 1.85 |
| 10 min | 1.52 | 3.61 | 2.08 |
| 15 min | 1.57 | 3.56 | 2.04 |
| 20 min | 1.42 | 3.53 | 2.10 |
| 25 min | 1.57 | 3.57 | 2.11 |
| 30 min | 1.54 | 3.55 | 2.10 |

### Example 5: Calcium Release from Organic Matcha Green Tea

In this example calcium release from organic Matcha green tea was investigated. Six samples of organic Matcha Love stone ground powder tea were prepared according to the conditions described in the Table 12 below. The fluoride release was measured at 5 minutes intervals for 30 minutes as summarized in Table 13 below as also illustrated in FIG. 8.

TABLE 12
Sample I: 1 g Matcha-Love Organic in 60 ml DIW, 150 RPM at room
temp.
Sample II: 1 g Matcha-Love Organic in 60 ml DIW, 250 RPM at room
temp.
Sample III: 1 g Matcha-Love Organic in 60 ml DIW, 400 RPM at room
temp.
Sample IV: 2 g Matcha-Love Organic in 60 ml DIW, 150 RPM at room
temp.
Sample V: 2 g Matcha-Love Organic in 60 ml DIW, 250 RPM at room
temp.
Sample VI: 2 g Matcha-Love Organic in 60 ml DIW, 400 RPM at room
temp.

TABLE 13

| | Sample | Sample | Sample | Sample | Sample | Sample |
|---|---|---|---|---|---|---|
| Time | I | II | III | IV | V | VI |
| 0 min | 0.18 | 0.15 | 0.17 | 0.18 | 0.17 | 0.15 |
| 5 min | 0.46 | 0.39 | 0.63 | 0.38 | 0.59 | 0.55 |
| 10 min | 0.35 | 0.36 | 0.39 | 0.27 | 0.51 | 0.46 |
| 15 min | 0.38 | 0.40 | 0.52 | 0.14 | 0.42 | 0.41 |
| 20 min | 0.38 | 0.46 | 0.44 | 0.25 | 0.44 | 0.42 |
| 25 min | 0.43 | 0.37 | 0.38 | 0.22 | 0.49 | 0.43 |
| 30 min | 0.36 | 0.37 | 0.48 | 0.22 | 0.37 | 0.38 |

### Example 6: Calcium Release from Organic Matcha Green Tea

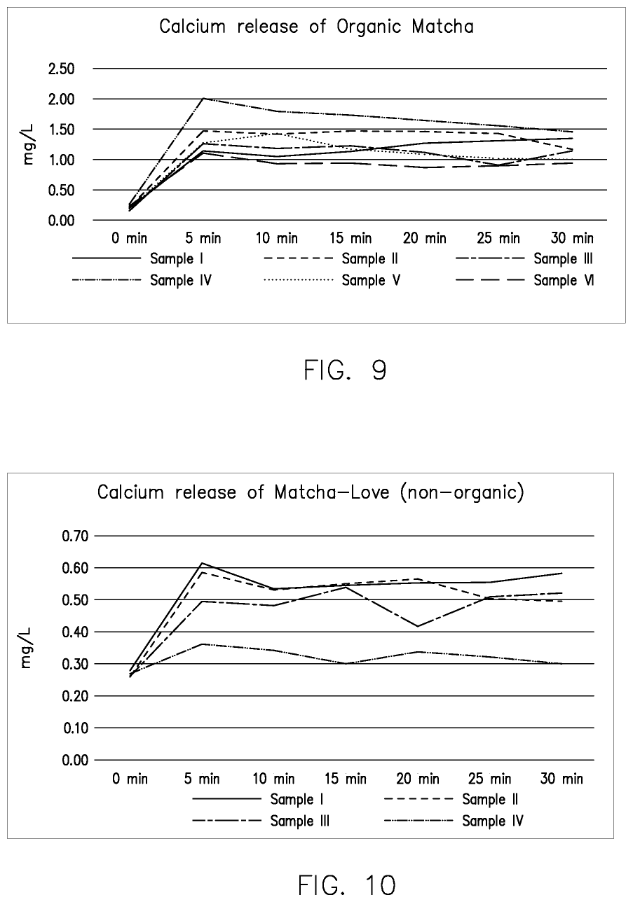

In this example calcium release from organic Matcha green tea was investigated. Six samples of organic Matcha powder tea available from Navitas Organics were dissolved in deionized water (DIW) according to the conditions described in the Table 14 below. The calcium release was measured at 5 minutes intervals for 30 minutes as summarized in Table 15 below as also illustrated in FIG. 9.

TABLE 14
Sample I: 1 g Organic Matcha in 60 ml DIW, 150 RPM at room temp.
Sample II: 1 g Organic Matcha in 60 ml DIW, 250 RPM at room temp.
Sample III: 1 g Organic Matcha in 60 ml DIW, 400 RPM at room temp.
Sample IV: 2 g Organic Matcha in 60 ml DIW, 150 RPM at room temp.
Sample V: 2 g Organic Matcha in 60 ml DIW, 250 RPM at room temp.
Sample VI: 2 g Organic Matcha in 60 ml DIW, 400 RPM at room temp.

TABLE 15

| | Sample | Sample | Sample | Sample | Sample | Sample |
|---|---|---|---|---|---|---|
| Time | I | II | III | IV | V | VI |
| 0 min | 0.18 | 0.16 | 0.15 | 0.27 | 0.21 | 0.24 |
| 5 min | 1.14 | 1.48 | 1.28 | 2.02 | 1.27 | 1.11 |
| 10 min | 1.05 | 1.42 | 1.18 | 1.81 | 1.43 | 0.93 |
| 15 min | 1.14 | 1.48 | 1.23 | 1.74 | 1.18 | 0.94 |
| 20 min | 1.28 | 1.46 | 1.12 | 1.65 | 1.09 | 0.87 |
| 25 min | 1.32 | 1.45 | 0.90 | 1.57 | 1.03 | 0.90 |
| 30 min | 1.35 | 1.19 | 1.14 | 1.46 | 1.00 | 0.95 |

### Example 7: Calcium Release from Non-Organic Matcha Green Tea

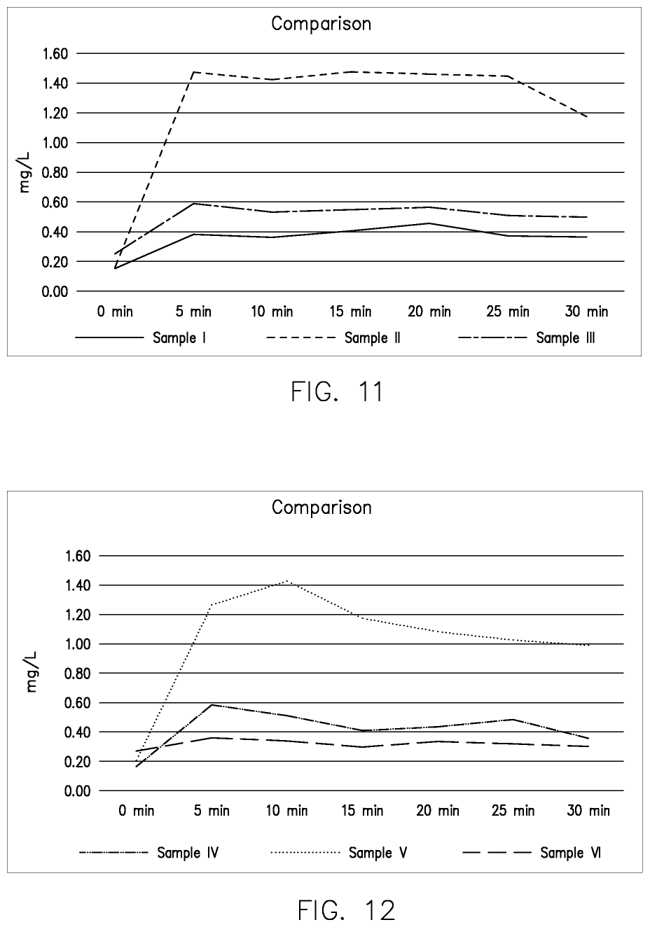

This study investigated fluoride release from non-organic Matcha green tea. Four samples of non-organic Matcha Love stone ground powder tea available from Usucha were prepared according to the conditions described in the Table 16 below. The fluoride release was measured at 5 minutes intervals for 30 minutes as summarized in Table 17 below as also illustrated in FIG. 10.

TABLE 16
Sample I: 1 g Matcha-Love (non-organic) in 60 ml DIW, 150 RPM at
room temp.
Sample II: 1 g Matcha-Love (non-organic) in 60 ml DIW, 250 RPM at
room temp.
Sample III: 1 g Matcha-Love (non-organic) in 60 ml DIW, 400 RPM at
room temp.
Sample IV: 2 g Matcha-Love (non-organic) in 60 ml DIW, 250 RPM at
room temp.

TABLE 17

| Time | Sample I | Sample II | Sample III | Sample IV |
|---|---|---|---|---|
| 0 min | 0.28 | 0.26 | 0.26 | 0.27 |
| 5 min | 0.62 | 0.59 | 0.50 | 0.36 |
| 10 min | 0.54 | 0.53 | 0.48 | 0.34 |
| 15 min | 0.55 | 0.55 | 0.54 | 0.30 |
| 20 min | 0.56 | 0.57 | 0.42 | 0.34 |
| 25 min | 0.55 | 0.51 | 0.51 | 0.32 |
| 30 min | 0.58 | 0.50 | 0.52 | 0.30 |

### Example 8: Comparison Among Different Types of Matcha Green Tea and their Calcium Release

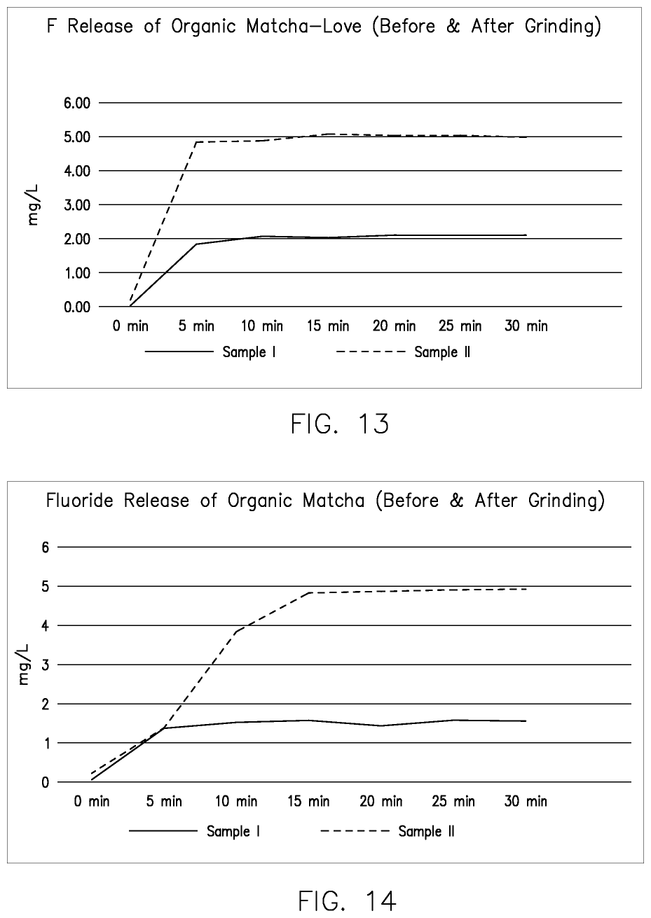

In this example different types of Matcha green tea and their calcium release were compared. The samples were prepared according to the conditions summarized in Tables 18 and 20. The comparison results are outlined in Tables 19 and 21 and illustrated in FIGS. 11 and 12.

TABLE 18
Sample I: 1 g Organic Matcha-Love in 60 ml DIW, 250 RPM at room
temp.
Sample II: 1 g Organic Matcha-Love in 60 ml DIW, 250 RPM at room
temp.
Sample III: 1 g Organic Matcha-Love in 60 ml DIW, 250 RPM at room
temp.

TABLE 19

| Time | Sample 1 | Sample 2 | Sample 3 |
|---|---|---|---|
| 0 min | 0.15 | 0.16 | 0.26 |
| 5 min | 0.39 | 1.48 | 0.59 |
| 10 min | 0.36 | 1.42 | 0.53 |
| 15 min | 0.40 | 1.48 | 0.55 |
| 20 min | 0.46 | 1.46 | 0.57 |
| 25 min | 0.37 | 1.45 | 0.51 |
| 30 min | 0.37 | 1.19 | 0.50 |

### Example 9: Fluoride Release of Organic Matcha Green Tea Before and After Grinding

In this example fluoride release from organic Matcha green tea was investigated before and after grinding. Grinding was done manually using a mortar and pestle for 10 minutes. Two samples of organic Matcha-Love powder tea were dissolved in deionized water (DIW) according to the conditions described in the Table 22 below. The fluoride release was measured at 5 minutes intervals for 30 minutes as summarized in Table 23 below as also illustrated in FIG. 13.

Table 22

Sample I: 2 g Organic Matcha-Love in 60 ml DIW, 250 RPM at room temperature.
Sample II: 2 g manually grinded Organic Matcha-Love in 60 ml DIW, 250 RPM at room temperature.

TABLE 23

| Time | Sample I | Sample II |
|---|---|---|
| 0 min | 0.05 | 0.21 |
| 5 min | 1.85 | 4.86 |
| 10 min | 2.08 | 4.88 |
| 15 min | 2.04 | 5.08 |
| 20 min | 2.10 | 5.04 |
| 25 min | 2.11 | 5.07 |
| 30 min | 2.10 | 4.99 |

### Example 10: Fluoride Release of Organic Matcha Green Tea Before and After Grinding

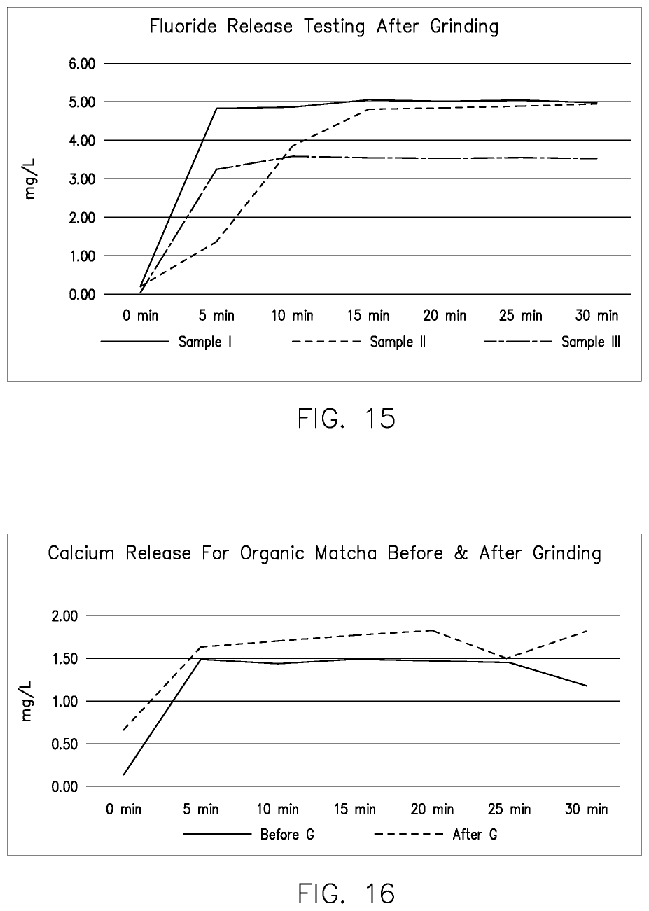

In this example fluoride release from organic Matcha green tea was investigated before and after grinding. Grinding was done manually using a mortar and pestle for 10 minutes. Two samples of organic Matcha powder tea were dissolved in deionized water (DIW) according to the conditions described in the Table 24 below. The fluoride release was measured at 5 minutes intervals for 30 minutes as summarized in Table 25 below as also illustrated in FIG. 14.

Table 24

Sample I: 2 g Organic Matcha in 60 ml DIW, 250 RPM at room temperature.
Sample II: 2 g manually grinded Organic Matcha, 60 ml DIW, 250 RPM, room temperature.

TABLE 25

| Time | Sample I | Sample II |
|---|---|---|
| 0 min | 0.08 | 0.22 |
| 5 min | 1.37 | 1.337 |
| 10 min | 1.52 | 3.86 |
| 15 min | 1.57 | 4.82 |
| 20 min | 1.42 | 4.87 |
| 25 min | 1.57 | 4.91 |
| 30 min | 1.54 | 4.94 |

### Example 11: Comparison of Fluoride Release of Organic Matcha Green Tea Before and After Grinding

In this example different types of Matcha green tea and their fluoride release before and after grinding were compared. The samples were prepared according to the conditions summarized in Table 26. The comparison results are outlined in Table 27 and illustrated in FIG. 15.

TABLE 26
Sample I: 2 g manually grinded Organic Matcha-Love in 60 ml DIW, 250
RPM at room temp.
Sample II: 2 g manually grinded Organic Matcha in 60 ml DIW, 250 RPM
at room temp.
Sample III: 2 g Non-Organic Matcha-Love (light bodied) in 60 ml DIW,
250 RPM at room temp.

TABLE 27

| Time | Sample 1 | Sample 2 | Sample 3 |
|---|---|---|---|
| 0 min | 0.21 | 0.22 | 0.06 |
| 5 min | 4.86 | 1.37 | 3.25 |
| 10 min | 4.88 | 3.86 | 3.61 |
| 15 min | 5.08 | 4.82 | 3.56 |
| 20 min | 5.04 | 4.87 | 3.53 |
| 25 min | 5.07 | 4.91 | 3.57 |
| 30 min | 4.99 | 4.94 | 3.55 |

### Example 12: Calcium Release of Organic Matcha Green Tea Before and After Grinding

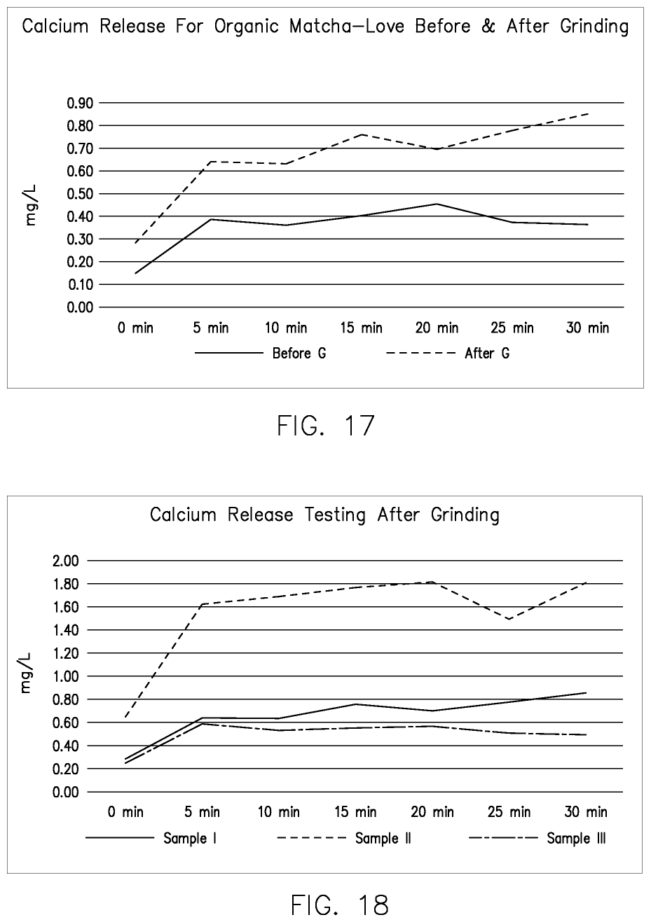

In this example calcium release from organic Matcha green tea was investigated before and after grinding. Grinding was done manually using a mortar and pestle for 10 minutes. Two samples of organic Matcha green tea were dissolved in deionized water (DIW) according to the conditions described in the Table 28 below, wherein G refers to grinding. The calcium release was measured at 5 minutes intervals for 30 minutes as summarized in Table 29 below as also illustrated in FIG. 16.

TABLE 28

Before G.: 1 g Organic Matcha in 60 ml DIW, 250 RPM at room
temp.
After G.: 1 g manually grinded Organic Matcha in 60 ml DIW, 250
RPM at room temp.

TABLE 29

| Time | Before G. | After G. |
|---|---|---|
| 0 min | 0.16 | 0.65 |
| 5 min | 1.48 | 1.63 |
| 10 min | 1.42 | 1.70 |
| 15 min | 1.48 | 1.77 |
| 20 min | 1.46 | 1.82 |
| 25 min | 1.45 | 1.50 |
| 30 min | 1.19 | 1.80 |

### Example 13: Calcium Release of Organic Matcha Green Tea Before and After Grinding

In this example calcium release from organic Matcha-Love tea was investigated before and after grinding. Grinding was done manually using a mortar and pestle for 10 minutes. Two samples of organic Matcha-Love tea were dissolved in deionized water (DIW) according to the conditions described in the Table 30 below, wherein G refers to grinding. The calcium release was measured at 5 minutes intervals for 30 minutes as summarized in Table 31 below as also illustrated in FIG. 17.

TABLE 30
Before G.: 1 g Organic Matcha-Love in 60 ml DIW, 250 RPM at room
temp.
After G.: 1 g manually grinded Organic Matcha-Love in 60 ml DIW, 250
RPM at room temp.

TABLE 31

| Time | Before G. | After G. |
|---|---|---|
| 0 min | 0.15 | 0.29 |
| 5 min | 0.39 | 0.64 |
| 10 min | 0.36 | 0.63 |
| 15 min | 0.40 | 0.76 |
| 20 min | 0.46 | 0.70 |
| 25 min | 0.37 | 0.78 |
| 30 min | 0.37 | 0.85 |

### Example 14: Comparison of Calcium Release of Organic and Non-Organic Matcha Green Tea Before and After Grinding

In this example different types of Matcha green tea and their calcium release before and after grinding were compared. The samples were prepared according to the conditions summarized in Table 32. We note that while the organic Matcha green tea was manually ground using a mortar and pestle, the non-organic powder tea of Sample III was not ground because it already had very fine particle size. The comparison results are outlined in Table 33 and illustrated in FIG. 18.

TABLE 32
Sample I: 1 g manually-grinded Organic Matcha-Love in 60 ml DIW,
250 RPM at room temp.
Sample II: 1 g manually-grinded Organic Matcha in 60 ml DIW, 250
RPM at room temp.
Sample III: 1 g Non-Organic Matcha-Love (light bodied) in 60 ml DIW,
250 RPM at room temp.

TABLE 33

| Time | Sample I | Sample II | Sample III |
|---|---|---|---|
| 0 min | 0.29 | 0.65 | 0.26 |
| 5 min | 0.64 | 1.63 | 0.59 |
| 10 min | 0.63 | 1.70 | 0.53 |
| 15 min | 0.76 | 1.77 | 0.55 |
| 20 min | 0.70 | 1.82 | 0.57 |
| 25 min | 0.78 | 1.50 | 0.51 |
| 30 min | 0.85 | 1.80 | 0.50 |

### Example 15: Introducing Organic Matcha into Lozenge's Inner Core

After comparing different types of Matcha green tea regarding their calcium and fluoride release, organic Matcha green tea which had the highest fluoride and calcium release after reducing its particle size by grinding was introduced into lozenge's inner core. In this example, the core of one lozenge weighs 3.1366 g. The composition of the core of the lozenge was:

| | | |
|---|---|---|
| 1- Xylitol | 2.5964 g | (82.77%) |
| 2- Kollidon CL | 0.5201 g | (16.58%) |
| 3- Glycerin | 0.02 g | (0.63%) |

The results of 6 trials are summarized below:

| 1^{st} Trial: At 150° C., 450 RPM | | |
|---|---|---|
| 1- Organic Matcha | 0.69 g | (22%) |
| 2- Xylitol | 1.881 g | (60%) |
| 3- Kollidon CL | 0.5201 | (16.58%) |
| 4- Glycerin | 0.02 g | (0.63%) |

* It congealed once we started to add Organic Matcha powder.

| 2^{nd} Trial: At 200° C., 450 RPM | | |
|---|---|---|
| 1- Organic Matcha | 0.3763 g | (12%) |
| 2- Xylitol | 2.1956 g | (70%) |
| 3- Kollidon CL | 0.5201 g | (16.58%) |
| 4- Glycerin | 0.02 g | (0.63%) |

* It congealed once Organic Matcha was added.

3^{rd} Trial: At 200° C., 450 RPM
* Repeat the 1^{st} trial but divide organic Matcha (0.69 g) into 4
quarters & add every quarter separately.
* We were able to add ONLY the first quarter of Organic Matcha
0.1725 g (6.65%) & make 10 samples.
* The mixture had a very thick consistency & a glass rod was used
to fill the mold with the formed dough.
* Total masses 21.077 g, Sample Avg. 2.107 g, Loss % 18.7%

4^{th} Trial: At 200° C., 450 RPM

| | | |
|---|---|---|
| 1- Organic Matcha | 0.313 g | (10%) |
| 2- Xylitol | 2.282 g | (72.77%) |
| 3- Kollidon CL | 0.5201 g | (16.58%) |
| 4- Glycerin | 0.02 g | (0.63%) |

* We were able to add the total amount of Organic Matcha however
it turned to be a dough.
* The formed dough had enough working time that allowed me to
make 12 samples & a glass rod was used to fill the mold.
* Organic Matcha ruined at 200° C. Its color turned into brown and
started to produce a smoke.
* Total masses 25.6923 g, Sample Avg. 2.141 g, Loss % 18%

| 5^{th} Trial: At 150° C., 450 RPM | | |
|---|---|---|
| 1- Organic Matcha | 0.1568 g | (5%) |
| 2- Xylitol | 2.4393 g | (77.77%) |
| 3- Kollidon CL | 0.5201 g | (16.58%) |
| 4- Glycerin | 0.02 g | (0.63%) |

* The same as the 4^{th} trial, but 10 samples were made.
* Total masses: 25.459 g, Sample avg. 2.5459 g, Loss % 18.8%

6^{th} Trial: At 150° C., 450 RPM

| | | |
|---|---|---|
| 1- Organic Matcha | 0.094 g | (3%) |
| 2- Xylitol | 2.5 g | (79.77%) |
| 3- Kollidon CL | 0.5201 g | (16.58%) |
| 4- Glycerin | 0.02 g | (0.63%) |

* We tried to pour it however it became a thick paste before reaching
the mold.
* A metallic spatula was used to fill the mold with the formed paste.
10 samples were made.
* Total masses 25.9008 g, Sample Avg. 2.59008 g, Loss % 17.3%.

### Example 16: Preparing Lozenge Core without Fluoride

In this study we investigated adding 5% organic Matcha to the original inner core composition, removing NaF from original inner core composition and making Matcha inner cores and fluoride free inner cores without fluoride as illustrate in Table 34 below for batches Nos. 3 and 4:

TABLE 34

| 1- Xylitol | 28.258 | g |
|---|---|---|
| 2- Kollidon CL | 5.654 | g |
| 3- Glycerin | 0.22 | g (7 drops) |
| 4- Licorice Extract | 0.011 | g |
| 5- KH2PO4 | 0.011 | g |
| 6- CaCl2 | 0.015 | g |
| 7- Green Tea Extract | 0.011 | g |
| 8- Casein P.P. | 0.703 | g |
| 9- Eucalyptus oil | 0.0319 | g (3 drops) |
| 10- Mango | 0.0247 | g (2 drops) |
| 11 samples were poured into a mold. | | |
| Total masses: | 27.385 | g |
| Sample Avg.: | 2.489 | g |
| Loss %: | 21.1% | |
| 2^{nd} run was named: Batch No. 4 | | |
| Composition: Has the same composition as batch No. 3 | | |
| 11 samples were poured into a mold. | | |
| Total masses: | 26.659 | g |
| Sample Avg.: | 2.423 | g |

| | |
|---|---|
| Loss %: | 23.2% |

In Table 35 we summarize the results of making inner lozenge cores without NaF obtained for batches Nos. 5 and 6:

TABLE 35
1^{St} run was named: Batch No. 5
Composition:

| | | |
|---|---|---|
| 1. 1-Xylitol | 26.498 | g |
| 2. Kollidon CL | 5.654 | g |
| 3. Glycerin | 0.22 | g (7 drops) |
| 4. Licorice Extract | 0.011 | g |
| 5. KH2PO4 | 0.011 | g |
| 6. CaCl2 | 0.015 | g |
| 7. Green Tea Extract | 0.011 | g |
| 8. Casein P.P. | 0.703 | g |
| 9. Eucalyptus oil | 0.0319 | g (3 drops) |
| 10. Organic Matcha | 1.749 | g |

Once we started to add Organic Matcha, the mix turned into a thick paste.
We had sufficient working time to fill a mold and make 12 samples.

| | | |
|---|---|---|
| Total masses: | 29.967 | g |
| Sample Avg.: | 2.497 | g |

| | |
|---|---|
| Loss %: | 14.1% |

2^{nd} run was named: Batch No. 6
Composition: Has the same composition as batch No. 5
12 samples were made.

| | | |
|---|---|---|
| Total masses: | 30.602 | g |
| Sample Avg.: | 2.550 | g |

| | |
|---|---|
| Loss %: | 12.3% |

### Example 17: pH of Organic Matcha Inner Core

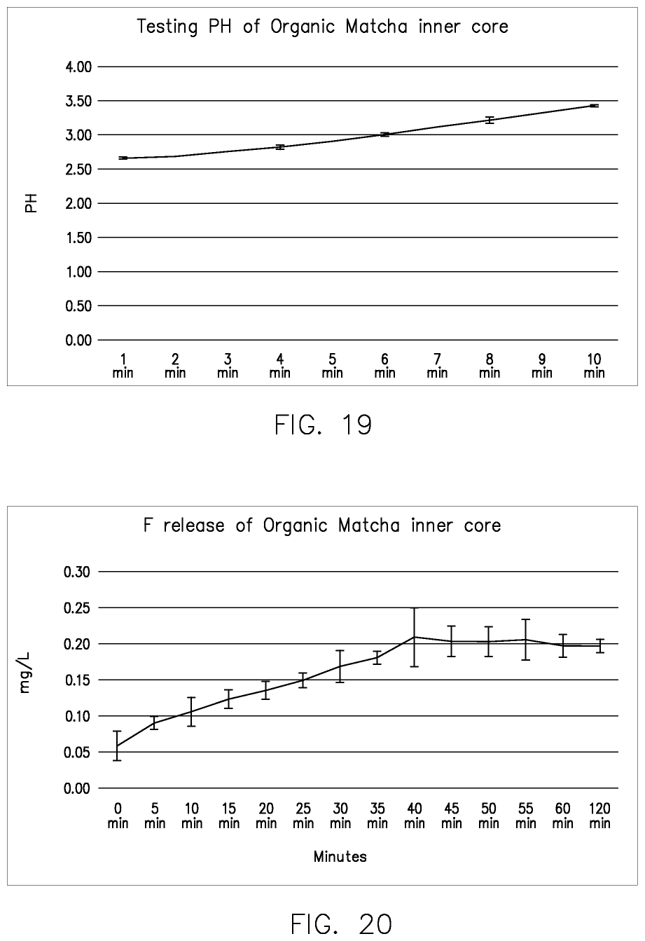

In this example, pH of organic Matcha inner core was studied in samples of batch No. 5 inner cores. The methodology included stirring which was performed using an 8×22 mm stir bar at 250 rpm and using 20 ml 0.01 HCl having pH of 1.53 in a 50 ml glass beaker for each sample. Table 36 summarizes the results which are also illustrated in FIG. 19.

TABLE 36

| Time | Sample 1 | Sample 2 | Sample 3 | AVERAGE | STDEV |
|---|---|---|---|---|---|
| 1 min | 2.66 | 2.67 | 2.66 | 2.66 | 0.01 |
| 2 min | 2.67 | 2.69 | 2.70 | 2.69 | 0.02 |
| 3 min | 2.75 | 2.77 | 2.77 | 2.76 | 0.01 |
| 4 min | 2.80 | 2.86 | 2.81 | 2.82 | 0.03 |
| 5 min | 2.90 | 2.92 | 2.90 | 2.91 | 0.01 |
| 6 min | 3.03 | 3.01 | 3.01 | 3.02 | 0.01 |
| 7 min | 3.13 | 3.11 | 3.12 | 3.12 | 0.01 |
| 8 min | 3.26 | 3.18 | 3.23 | 3.22 | 0.04 |
| 9 min | 3.32 | 3.34 | 3.33 | 3.33 | 0.01 |
| 10 min | 3.42 | 3.43 | 3.43 | 3.43 | 0.01 |

### Example 18: Fluoride Release of Organic Matcha Inner Core

In this example the fluoride release of organic Matcha inner core was studied on samples of batch No. 5 inner cores. The methodology included completely dissolving sample 1, 2 and 3 after 55, 60 and 45 minutes, respectively. The results are summarized in Table 37 and illustrated in FIG. 20.

TABLE 37

| Time | | Sample 1 | Sample 2 | Sample 3 | AVERAGE | STDEV |
|---|---|---|---|---|---|---|
| 0 | min | 0.07 | 0.07 | 0.04 | 0.06 | 0.02 |
| 5 | min | 0.10 | 0.08 | 0.09 | 0.09 | 0.01 |
| 10 | min | 0.09 | 0.13 | 0.10 | 0.11 | 0.02 |
| 15 | min | 0.14 | 0.12 | 0.12 | 0.12 | 0.01 |
| 20 | min | 0.15 | 0.13 | 0.13 | 0.14 | 0.01 |
| 25 | min | 0.16 | 0.14 | 0.15 | 0.15 | 0.01 |
| 30 | min | 0.19 | 0.16 | 0.15 | 0.17 | 0.02 |
| 35 | min | 0.19 | 0.18 | 0.18 | 0.18 | 0.01 |
| 40 | min | 0.21 | 0.25 | 0.17 | 0.21 | 0.04 |
| 45 | min | 0.22 | 0.22 | 0.18 | 0.20 | 0.02 |
| 50 | min | 0.22 | 0.21 | 0.18 | 0.20 | 0.02 |
| 55 | min | 0.24 | 0.20 | 0.18 | 0.21 | 0.03 |
| 60 | min | 0.22 | 0.19 | 0.19 | 0.20 | 0.02 |
| 120 | min | 0.20 | 0.20 | 0.19 | 0.20 | 0.01 |

### Example 19: Calcium Release of Organic Matcha Inner Core

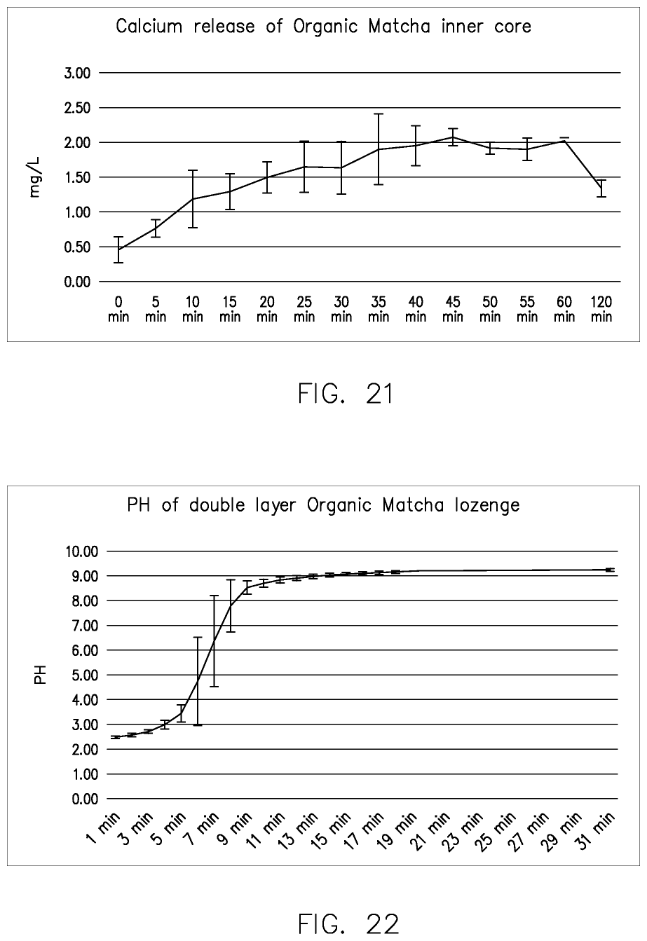

In this example the calcium release of organic Matcha inner core was studied on samples of batch No. 5 inner cores. The methodology included completely dissolving sample 1 and 2 after 60 minutes. Sample 3 was completely dissolved after 70 minutes. The results are summarized in Table 38 and illustrated in FIG. 21.

TABLE 38

| Time | | Sample 1 | Sample 2 | Sample 3 | AVERAGE | STDEV |
|---|---|---|---|---|---|---|
| 0 | min | 0.67 | 0.34 | 0.37 | 0.46 | 0.18 |
| 5 | min | 0.81 | 0.86 | 0.62 | 0.76 | 0.13 |
| 10 | min | 1.66 | 0.95 | 0.94 | 1.18 | 0.41 |
| 15 | min | 1.30 | 1.54 | 1.03 | 1.29 | 0.26 |
| 20 | min | 1.39 | 1.75 | 1.33 | 1.49 | 0.23 |
| 25 | min | 1.81 | 1.91 | 1.23 | 1.65 | 0.37 |
| 30 | min | 1.52 | 2.06 | 1.32 | 1.63 | 0.38 |
| 35 | min | 1.84 | 2.44 | 1.42 | 1.90 | 0.51 |
| 40 | min | 1.94 | 2.25 | 1.69 | 1.96 | 0.28 |
| 45 | min | 1.98 | 2.22 | 2.04 | 2.08 | 0.12 |
| 50 | min | 1.83 | 2.00 | 1.93 | 1.92 | 0.09 |
| 55 | min | 2.05 | 1.93 | 1.73 | 1.90 | 0.16 |
| 60 | min | 2.06 | 2.01 | 2.05 | 2.04 | 0.03 |
| 120 | min | 1.35 | 1.46 | 1.21 | 1.34 | 0.13 |

### Example 20: Adherence of Lozenge's Outer Layer to the Organic Matcha Inner Core

In this example the adherence of the outer layer of a lozenge to the organic Matcha inner core was tested. The composition of the inner core of batch Nos. 7-1 and 7-2 is summarized in Table 39. The compositions of the outer layer for these batches are outlined in Table 40.

TABLE 39

| 1^{st} Run, Named batch No. 7-1 | | |
|---|---|---|
| Composition: | | |
| Theoretically, this composition should make 10 samples. | | |
| 1. Xylitol | 26.498 | g |
| 2. Kollidon CL | 5.654 | g |
| 3. Glycerin | 0.22 | g (7 drops) |
| 4. Licorice Extract | 0.011 | g |
| 5. KH2PO4 | 0.011 | g |
| 6. CaCl2 | 0.015 | g |
| 7. Green Tea Extract | 0.011 | g |
| 8. Casein P.P. | 0.703 | g |
| 9. Eucalyptus oil | 0.0319 | g (3 drops) |
| 10. Organic Matcha | 1.749 | g |
| 12 samples were made from the previous composition. | | |
| Total masses: | 31.846 | g |
| Sample Avg.: | 2.653 | g |
| Loss %: | 8.75% | |
| 2^{nd} Run, Named batch No. 7-2 | | |
| Composition: Has the composition of batch No. 7-1 | | |
| 12 samples were poured. | | |
| Total masses: | 31.317 | g |
| Sample Avg.: | 2.609 | g |

| | |
|---|---|
| Loss %: | 10.27% |

TABLE 40

| Composition: | |
|---|---|
| 1- Xylitol | 28.561 g |
| 2- Kollidon CL | 5.721 g |
| 3- Glycerin | (7 drops) |
| 4- Green Tea Extract | 0.0125 g |
| 5- Eucalyptus oil | (3 drops) |
| 6- Arginine | 0.352 g |

The previous composition was used to make six double layer organic Matcha lozenges. After letting the outer layer congeal, we found that the lozenge's outer layer adhered perfectly to the organic Matcha inner core.

The methodology of preparing an organic Matcha inner core of a lozenge having triple calcium and phosphate content based on Table 39 included placing xylitol in a 250 ml glass beaker and adjust heat at 150° C. until the xylitol was completely melted and turned into a clear liquid. 5.654 g Kollidon CL was added and melted into the liquid xylitol until a homogeneous mixture was obtained. About 7 drops of glycerin were added. The remaining components of licorice extract, green tea extract, KH2PO4, Matcha, eucalyptus oil, casein phosphopeptide, mango flavor, CaCl2 were added with mixing and heat to ensure a homogeneous mixture. The organic Matcha was added all at once followed by manual mixing. The inner core mixture was then poured into a small mold that had been previously lubricated with canola or vegetable oil. The inner core was then allowed to congeal and then it is removed from the mold and immersed into the outer layer and allowed to congeal again.

The methodology for preparing the lozenge's outer layer included adding 28.561 g xylitol to a 250 mL glass container, heating the xylitol to a temperature of 150° C. until the xylitol became clear, followed by stirring the xylitol until it was melted. adding an amount of 5.721 g of Kollidon CL to the dissolved xylitol in the glass container to form a mixture. 7 drops of glycerin were added to the mixture, followed by sequentially adding an amount of green tea extract, an amount of licorice extract and then an amount eucalyptus oil to the mixture, quickly adding and mixing L-arginine as listed in Table 40 to the mixture with continued mixing to ensure a homogenous mixture. The resulting mixture was poured into the mold immediately after the L-arginine was mixed into the mixture, and the mixture was congealed at room temperature for a period time of from about 24 to about 48 hours to form the multi-layered lollipop.

### Example 21: pH of the Double Layer Organic Matcha Lozenges

In this example, the pH of the double layer organic Matcha lozenges prepared in Example 20 was investigated. The results are summarized in Table 41 and illustrated in FIG. 22. In this table readings with respect to sample 1 were taken after reaching a plateau. With respect to sample 2, readings were taken until the sample was completely dissolved after 40 minutes. With respect to sample 3, readings were taken until the sample completely dissolved after 31 minutes.

TABLE 41

| Time | | Sample 1 | Sample 2 | Sample 3 | AVERAGE | STDEV |
|---|---|---|---|---|---|---|
| 1 | min | 2.47 | 2.51 | 2.54 | 2.51 | 0.04 |
| 2 | min | 2.54 | 2.59 | 2.6 | 2.58 | 0.03 |
| 3 | min | 2.67 | 2.69 | 2.79 | 2.72 | 0.06 |
| 4 | min | 2.93 | 2.88 | 3.2 | 3.00 | 0.17 |
| 5 | min | 3.27 | 3.24 | 3.85 | 3.45 | 0.34 |
| 6 | min | 3.82 | 3.62 | 6.77 | 4.74 | 1.76 |
| 7 | min | 5.9 | 4.79 | 8.38 | 6.36 | 1.84 |
| 8 | min | 8.13 | 6.61 | 8.64 | 7.79 | 1.06 |
| 9 | min | 8.5 | 8.29 | 8.78 | 8.52 | 0.25 |
| 10 | min | 8.67 | 8.57 | 8.88 | 8.71 | 0.16 |
| 11 | min | 8.82 | 8.71 | 8.95 | 8.83 | 0.12 |
| 12 | min | 8.89 | 8.82 | 9.01 | 8.91 | 0.10 |
| 13 | min | 8.97 | 8.9 | 9.06 | 8.98 | 0.08 |
| 14 | min | 9.01 | 9.01 | 9.1 | 9.04 | 0.05 |
| 15 | min | 9.07 | 9.06 | 9.13 | 9.09 | 0.04 |
| 16 | min | 9.1 | 9.08 | 9.15 | 9.11 | 0.04 |
| 17 | min | | 9.12 | 9.17 | 9.15 | 0.04 |
| 18 | min | | 9.12 | 9.18 | 9.15 | 0.04 |
| 19 | min | | 9.19 | 9.2 | 9.20 | 0.01 |
| 20 | min | | 9.21 | 9.21 | 9.21 | 0.00 |
| 21 | min | | 9.21 | 9.22 | 9.22 | 0.00 |
| 22 | min | | 9.21 | 9.23 | 9.22 | 0.01 |
| 23 | min | | 9.21 | 9.23 | 9.22 | 0.01 |
| 24 | min | | 9.22 | 9.23 | 9.23 | 0.01 |
| 25 | min | | 9.22 | 9.24 | 9.23 | 0.01 |
| 26 | min | | 9.22 | 9.24 | 9.23 | 0.01 |
| 27 | min | | 9.23 | 9.23 | 9.23 | 0.00 |
| 28 | min | | 9.23 | 9.23 | 9.23 | 0.00 |
| 29 | min | | 9.23 | 9.23 | 9.23 | 0.00 |
| 30 | min | | 9.25 | 9.22 | 9.24 | 0.02 |
| 31 | min | | 9.27 | 9.22 | 9.25 | 0.04 |
| 32 | min | | 9.27 | | | |
| 33 | min | | 9.27 | | | |
| 34 | min | | 9.26 | | | |
| 35 | min | | 9.26 | | | |
| 36 | min | | 9.25 | | | |

### Example 22: pH of Organic Matcha Inner Core

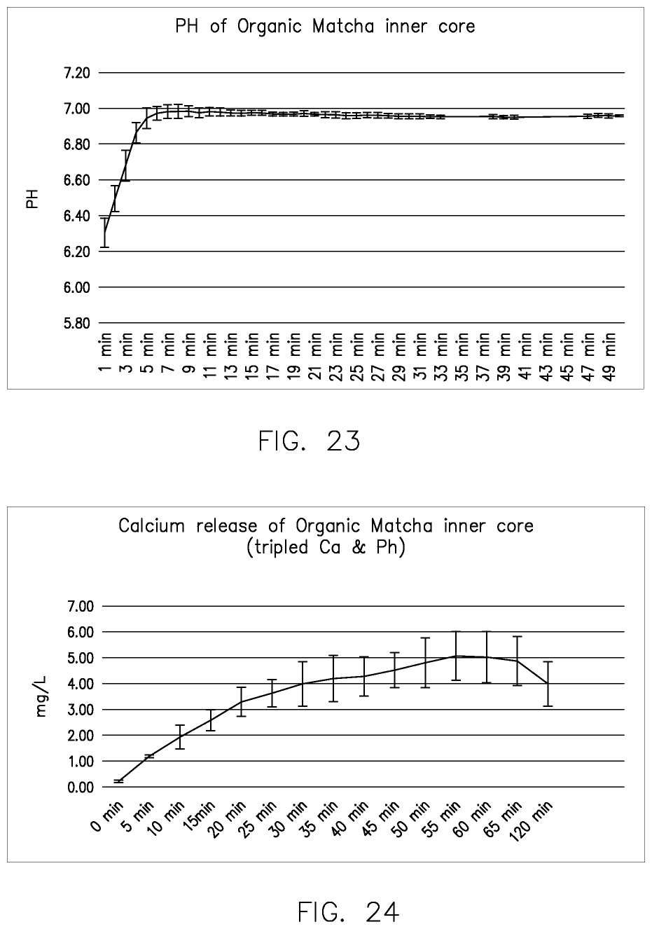

In this example the pH of the organic Matcha inner core of batch 7-1 was studied. The methodology included placing 60 ml deionized water (DIW) in a 250 ml glass beaker, adjusting the stir plate speed at 450 RPM, placing a stir bar in the center of the glass beaker, placing an inner core sample in the glass beaker and start a stopwatch and taking readings every 1 minute till the sample completely dissolved. The results are summarized in Table 42 and illustrated in FIG. 23.

TABLE 42

| Time | | Sample 1 | Sample 2 | Sample 3 | AVERAGE | STDEV |
|---|---|---|---|---|---|---|
| 1 | min | 6.22 | 6.31 | 6.38 | 6.30 | 0.08 |
| 2 | min | 6.41 | 6.53 | 6.54 | 6.49 | 0.07 |
| 3 | min | 6.58 | 6.73 | 6.73 | 6.68 | 0.09 |
| 4 | min | 6.80 | 6.88 | 6.91 | 6.86 | 0.06 |
| 5 | min | 6.88 | 6.96 | 6.99 | 6.94 | 0.06 |
| 6 | min | 6.93 | 6.99 | 7.00 | 6.97 | 0.04 |
| 7 | min | 6.94 | 7.00 | 7.01 | 6.98 | 0.04 |
| 8 | min | 6.94 | 7.00 | 7.01 | 6.98 | 0.04 |
| 9 | min | 6.95 | 6.99 | 7.01 | 6.98 | 0.03 |
| 10 | min | 6.95 | 6.98 | 7.00 | 6.98 | 0.03 |
| 11 | min | 6.96 | 6.98 | 7.01 | 6.98 | 0.03 |
| 12 | min | 6.96 | 6.98 | 7.00 | 6.98 | 0.02 |
| 13 | min | 6.96 | 6.98 | 6.99 | 6.98 | 0.02 |
| 14 | min | 6.96 | 6.97 | 6.99 | 6.97 | 0.02 |
| 15 | min | 6.97 | 6.97 | 6.99 | 6.98 | 0.01 |
| 16 | min | 6.97 | 6.97 | 6.99 | 6.98 | 0.01 |
| 17 | min | 6.97 | 6.96 | 6.98 | 6.97 | 0.01 |
| 18 | min | 6.97 | 6.96 | 6.98 | 6.97 | 0.01 |
| 19 | min | 6.97 | 6.96 | 6.98 | 6.97 | 0.01 |
| 20 | min | 6.97 | 6.96 | 6.99 | 6.97 | 0.02 |
| 21 | min | 6.97 | 6.96 | 6.98 | 6.97 | 0.01 |
| 22 | min | 6.97 | 6.95 | 6.98 | 6.97 | 0.02 |
| 23 | min | 6.98 | 6.95 | 6.97 | 6.97 | 0.02 |
| 24 | min | 6.98 | 6.95 | 6.95 | 6.96 | 0.02 |
| 25 | min | 6.98 | 6.95 | 6.95 | 6.96 | 0.02 |
| 26 | min | 6.98 | 6.96 | 6.95 | 6.96 | 0.02 |
| 27 | min | 6.98 | 6.96 | 6.95 | 6.96 | 0.02 |
| 28 | min | 6.97 | 6.96 | 6.95 | 6.96 | 0.01 |
| 29 | min | 6.97 | 6.95 | 6.95 | 6.96 | 0.01 |
| 30 | min | 6.97 | 6.95 | 6.95 | 6.96 | 0.01 |
| 31 | min | 6.97 | 6.95 | 6.95 | 6.96 | 0.01 |
| 32 | min | 6.96 | 6.95 | 6.95 | 6.95 | 0.01 |
| 33 | min | 6.96 | 6.95 | 6.95 | 6.95 | 0.01 |
| 34 | min | 6.96 | 6.95 | 6.95 | 6.95 | 0.01 |
| 35 | min | 6.95 | 6.95 | 6.95 | 6.95 | 0.00 |
| 36 | min | 6.96 | 6.95 | 6.95 | 6.95 | 0.01 |
| 37 | min | 6.96 | 6.95 | 6.95 | 6.95 | 0.01 |
| 38 | min | 6.96 | 6.95 | 6.96 | 6.96 | 0.01 |
| 39 | min | 6.96 | 6.95 | 6.95 | 6.95 | 0.01 |
| 40 | min | 6.96 | 6.95 | 6.95 | 6.95 | 0.01 |
| 41 | min | 6.96 | 6.95 | 6.95 | 6.95 | 0.01 |
| 42 | min | 6.95 | 6.95 | 6.95 | 6.95 | 0.00 |
| 43 | min | 6.95 | 6.95 | 6.95 | 6.95 | 0.00 |
| 44 | min | 6.96 | 6.95 | 6.95 | 6.95 | 0.01 |
| 45 | min | 6.96 | 6.95 | 6.95 | 6.95 | 0.01 |
| 46 | min | 6.95 | | 6.95 | 6.95 | 0.00 |
| 47 | min | 6.96 | | 6.95 | 6.96 | 0.01 |
| 48 | min | 6.96 | | 6.95 | 6.96 | 0.01 |
| 49 | min | 6.96 | | 6.95 | 6.96 | 0.01 |
| 50 | min | 6.96 | | 6.96 | 6.96 | 0.00 |

### Example 23: Calcium and Fluoride Release and Hardness of Matcha Lozenges Having Triple Calcium and Phosphate Content

In this example, the calcium and fluoride release and hardness of Matcha lozenges was tested. Based on the hardening test results obtained from testing the hardness of organic Matcha and fluoride free inner cores, samples having triple calcium and phosphate content of Matcha and fluoride free inner cores were prepared and tested for calcium release, fluoride release and hardness. The compositions of these samples are summarized in Tables 43 (1^{st} run) and 44 (2^{nd} run), respectively.

TABLE 43

| First Run Composition | | |
|---|---|---|
| 1. Xylitol | 26.446 | g |
| 2. Kollidon CL | 5.654 | g |
| 3. Glycerin | 0.22 | g (7 drops) |
| 4. Licorice Extract | 0.011 | g |
| 5. KH₂PO₄ | 0.033 | g |
| 6. CaCl₂ | 0.045 | g |
| 7. Green Tea Extract | 0.011 | g |
| 8. Casein P.P. | 0.703 | g |
| 9. Eucalyptus oil | 0.0319 | g (3 drops) |
| 10. Organic Matcha | 1.749 | g |
| Total masses: | 30.15 | g |
| Sample Avg.: | 2.512 | g |
| Loss %: | 13.6% | |
| Second Run Composition | | |
| Composition: the same as 1^{st} run | | |
| Total masses: | 31.63 | g |
| Sample Avg.: | 2.433 | g |

| | |
|---|---|
| Loss %: | 9.3% |

TABLE 44

| First Run Composition | | |
|---|---|---|
| 1. Xylitol | 28.203 | g |
| 2. Kollidon CL | 5.654 | g |
| 3. Glycerin | 0.22 | g (7 drops) |
| 4. Licorice Extract | 0.011 | g |
| 5. KH₂PO₄ | 0.033 | g |
| 6. CaCl2 | 0.045 | g |
| 7. Green Tea Extract | 0.011 | g |
| 8. Casein P.P. | 0.703 | g |
| 9. Eucalyptus oil | 0.0319 | g (3 drops) |
| 10. Mango flavor | 0.0247 | g (2 drops) |
| Total masses: | 27.784 | g |
| Sample Avg.: | 2.525 | g |
| Loss %: | 20.47% | |
| Second Run Composition | | |
| Composition: the same as 1^{st} run | | |
| Total masses: | 28.049 | g |
| Sample Avg.: | 2.549 | g |

| | |
|---|---|
| Loss %: | 19.7% |

The methodology of preparing an organic Matcha inner core of a lozenge having triple calcium and phosphate content based on Table 43 included placing xylitol in a 250 ml glass beaker and adjust heat at 150° C. until the xylitol was completely melted and turned into a clear liquid. 5.654 g Kollidon CL was added and melted into the liquid xylitol until a homogeneous mixture was obtained. About 7 drops of glycerin were added. The remaining components of licorice extract, green tea extract, KH₂PO₄, Matcha, eucalyptus oil, casein phosphopeptide, mango flavor, CaCl₂ were added with mixing and heat to ensure a homogeneous mixture. The organic Matcha was added all at once followed by manual mixing. The inner core mixture was then poured into a small mold that had been previously lubricated with canola or vegetable oil. The inner core was then allowed to congeal and then it is removed from the mold and immersed into the outer layer and allowed to congeal again.

### Example 24: Calcium Release of Organic Matcha Inner Core Having Tripled Calcium and Phosphate Content

In this example, the calcium release of organic Matcha green tea inner core samples having a content of tripled calcium and phosphate were tested. The results are summarized in Table 45 and illustrated in FIG. 24.

TABLE 45

| Time | | Sample 1 | Sample 2 | Sample 3 | AVERAGE | STDEV |
|---|---|---|---|---|---|---|
| 0 | min | 0.224 | 0.169 | 0.235 | 0.21 | 0.04 |
| 5 | min | 1.21 | 1.17 | 1.1 | 1.16 | 0.06 |
| 10 | min | 2.34 | 1.42 | 2.01 | 1.92 | 0.47 |
| 15 | min | 3.03 | 2.39 | 2.29 | 2.57 | 0.40 |
| 20 | min | 3.76 | 2.66 | 3.42 | 3.28 | 0.56 |
| 25 | min | 3.63 | 3.09 | 4.13 | 3.62 | 0.52 |
| 30 | min | 4.06 | 3.1 | 4.82 | 3.99 | 0.86 |
| 35 | min | 4.47 | 3.19 | 4.9 | 4.19 | 0.89 |
| 40 | min | 4.54 | 3.42 | 4.82 | 4.26 | 0.74 |
| 45 | min | 4.93 | 3.75 | 4.89 | 4.52 | 0.67 |
| 50 | min | 5.17 | 3.74 | 5.56 | 4.82 | 0.96 |
| 55 | min | 5.87 | 4.04 | 5.33 | 5.08 | 0.94 |
| 60 | min | 5.88 | 3.93 | 5.23 | 5.01 | 0.99 |
| 65 | min | 5.79 | 3.91 | 4.9 | 4.87 | 0.94 |
| 120 | min | 4.63 | 3.02 | 4.31 | 3.99 | 0.85 |

### Example 25: Fluoride Release of Organic Matcha Green Tea Inner Core Having Tripled Calcium and Phosphate Content

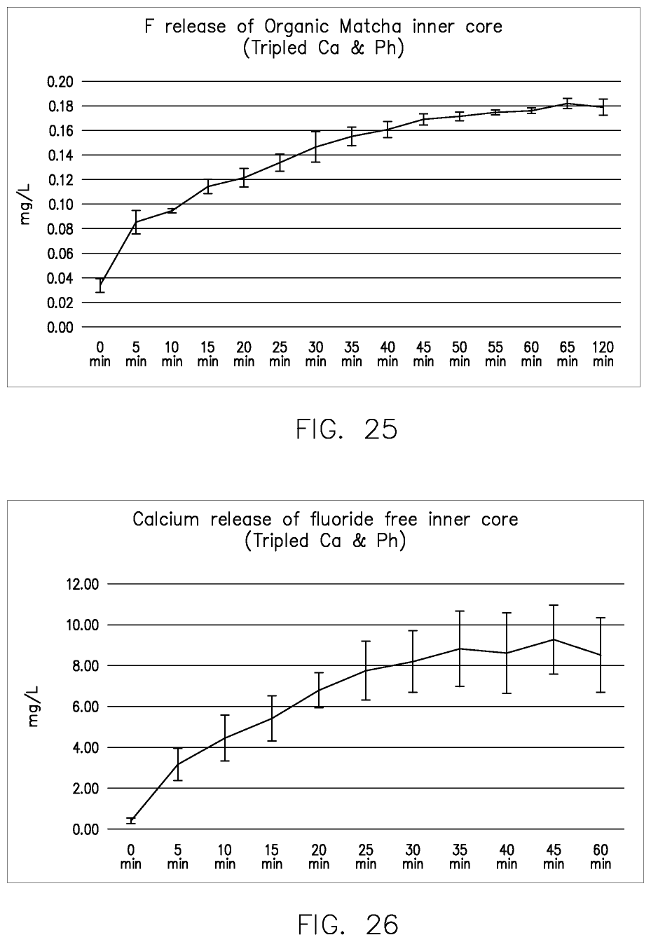

In this example the fluoride release of organic Matcha green tea inner core samples having a content of tripled calcium and phosphate was tested. The results are summarized in Table 46 and illustrated in FIG. 25.

TABLE 46

| Time | | Sample 1 | Sample 2 | Sample 3 | AVERAGE | STDEV |
|---|---|---|---|---|---|---|
| 0 | min | 0.04 | 0.03 | 0.04 | 0.03 | 0.01 |
| 5 | min | 0.08 | 0.08 | 0.10 | 0.09 | 0.01 |
| 10 | min | 0.09 | 0.10 | 0.09 | 0.09 | 0.00 |
| 15 | min | 0.11 | 0.11 | 0.12 | 0.11 | 0.01 |
| 20 | min | 0.13 | 0.12 | 0.12 | 0.12 | 0.01 |
| 25 | min | 0.14 | 0.13 | 0.14 | 0.13 | 0.01 |
| 30 | min | 0.16 | 0.14 | 0.15 | 0.15 | 0.01 |
| 35 | min | 0.16 | 0.15 | 0.15 | 0.16 | 0.01 |
| 40 | min | 0.17 | 0.15 | 0.16 | 0.16 | 0.01 |
| 45 | min | 0.17 | 0.16 | 0.17 | 0.17 | 0.00 |
| 50 | min | 0.17 | 0.18 | 0.17 | 0.17 | 0.00 |
| 55 | min | 0.17 | 0.17 | 0.18 | 0.18 | 0.00 |
| 60 | min | 0.17 | 0.18 | 0.18 | 0.18 | 0.00 |
| 65 | min | | 0.19 | 0.18 | 0.18 | 0.00 |
| 120 | min | 0.17 | 0.18 | 0.19 | 0.18 | 0.01 |

### Example 26: Fluoride Free Inner Core Having Tripled Calcium and Phosphate Content

In this example samples were prepared wherein the inner core was fluoride free. The composition of these samples is summarized in Table 47. The calcium release of these fluoride free inner cores having tripled calcium and phosphate content is summarized in Table 48 and illustrated in FIG. 26.

TABLE 47

| | | |
|---|---|---|
| Xylitol | 28.203 | g |
| Kollidon CL | 5.654 | g |
| Glycerin | 0.22 | g (7 drops) |
| Licorice Extract | 0.011 | g |
| KH₂PO₄ | 0.033 | g |
| CaCl₂ | 0.045 | g |
| Green Tea Extract | 0.011 | g |
| Casein P.P. | 0.703 | g |
| Eucalyptus oil | 0.0319 | g (3 drops) |
| Mango flavor | 0.0247 | g (2 drops |

Total masses:
Sample Avg.:
Loss %:

TABLE 48

| Time | Sample 1 | Sample 2 | Sample 3 | sample 6 | sample 7 | AVERAGE | STDEV |
|---|---|---|---|---|---|---|---|
| 0 min | 0.50 | 0.29 | 0.25 | 0.49 | 0.44 | 0.39 | 0.12 |
| 5 min | 3.64 | 3.01 | 3.85 | 1.85 | 3.43 | 3.16 | 0.79 |
| 10 min | 6.29 | 3.41 | 4.54 | 3.72 | 4.25 | 4.44 | 1.12 |
| 15 min | 6.90 | 4.22 | 6.11 | 4.48 | 5.35 | 5.41 | 1.12 |
| 20 min | 7.66 | 5.83 | 6.85 | 6.00 | 7.52 | 6.77 | 0.84 |
| 25 min | 10.10 | 7.55 | 7.16 | 6.20 | 7.73 | 7.75 | 1.44 |
| 30 min | 10.70 | 8.13 | 7.44 | 6.85 | 7.77 | 8.18 | 1.49 |
| 35 min | 12.00 | 8.18 | 8.55 | 7.22 | 8.17 | 8.82 | 1.84 |
| 40 min | 11.70 | 7.18 | 9.28 | 6.72 | 8.19 | 8.61 | 1.99 |
| 45 min | 11.70 | 7.88 | 9.15 | | 8.38 | 9.28 | 1.70 |
| 60 min | 11.20 | 7.65 | 9.19 | 6.31 | 8.26 | 8.52 | 1.83 |
| 120 min | 11.30 | | | | | | |

### Example 27: Calcium and Fluoride Release and Hardness of Matcha Lozenges Having Ten Times Calcium and Phosphate Content

In this example, the calcium and fluoride release and hardness of Matcha inner core was tested. Based on the hardening test results obtained from testing the hardness of organic Matcha and fluoride free inner cores having triple calcium and phosphate content, samples having ten times calcium and phosphate content of Matcha were prepared and tested for calcium release, fluoride release and hardness. Table 49 summarizes the composition of organic Matcha inner core containing ten times calcium and phosphate content.

TABLE 49

| | | |
|---|---|---|
| 1. Xylitol | 26.264 | g |
| 2. Kollidon CL | 5.654 | g |
| 3. Glycerin | 0.22 | g (7 drops) |
| 4. Licorice Extract | 0.011 | g |
| 5. KH2PO4 | 0.11 | g |
| 6. CaCl2 | 0.15 | g |
| 7. Green Tea Extract | 0.011 | g |
| 8. Casein P.P. | 0.703 | g |
| 9. Eucalyptus oil | 0.0319 | g (3 drops) |
| 10. Organic Matcha | 1.749 | g |

Table 50 summarizes results obtained by using the composition of table 49.

TABLE 50

| | [symbol] | | | | | |
|---|---|---|---|---|---|---|
| | Fluoride | | [symbol] | | [symbol] loz | |
| Composition/Sample | Inner Core | | Inner core | | Inner core | |
| Xylitol | 2.8237 g | Masses: | 2.8258 g | Masses: | 2.6498 g | Masses: |
| | | 1-2.494 g | | 1-2.669 g | | 1-2.528 g |
| Kollidon CL | 0.5645 g | 2-2.296 g | 0.5654 g | 2-2.581 g | 0.5654 g | 2-2.334 g |
| Glycerin | 0.022 g | 3-2.256 g | 0.022 g | 3-2.438 g | 0.022 g | 3-2.578 g |
| | (22 mg) | | (22 mg) | | (22 mg) | |
| NaF | 0.0016 g | 4-2.209 g | 0.00 g | 4-2.610 g | 0.00 g | 4-2.260 g |
| | (1.6 mg) | | | | | |
| KH2PO4 | 0.0011 g | 5-2.523 g | 0.0011 g | 5-2.754 g | 0.0011 g | 5-2.564 g |
| | (1.1 mg) | | (1.1 mg) | | (1.1 mg) | |
| CaCl2 | 0.0015 g | 6-2.089 g | 0.0015 g | 6-2.609 g | 0.0015 g | 6-2.649 g |
| | (1.5 mg) | | (1.5 mg) | | (1.5 mg) | |
| Licorice Ext. | 0.0011 g | 7-2.127 g | 0.0011 g | 7-2.257 g | 0.0011 g | 7-2.300 g |
| | (1.1 mg) | | (1.1 mg) | | (1.1 mg) | |
| Green Tea Extract | 0.0011 g | 8-3.087 g | 0.0011 g | 8-2.442 g | 0.0011 g | 8-2.631 g |
| | (1.1 mg) | | (1.1 mg) | | (1.1 mg) | |
| Casein P.P | 0.0704 g | 9-2.296 g | 0.0703 g | 9-2.368 g | 0.0703 g | 9-2.633 g |
| | (70.4 mg) | | (70.3 mg) | | (70.3 mg) | |
| Eucalyptus oil | 0.00319 g | 10-2.131 g | 0.00319 g | 10-2.414 g | 0.00319 g | 10-2.376 g |
| | (3.1 mg) | | (3.1 mg) | | (3.1 mg) | |
| Mango flavoring | 1/5 drop | | 1/5 drop | 11-2.243 g | 0.00 g | 11-2.556 g |
| Organic Matcha | 0.00 g | | 0.00 g | | 0.1749 g | 12-2.558 g |
| | | | | | (174 mg) | |
| Theoretically, total masses | 34.909 g | | 34.939 g | | 34.903 g | |
| of 10 samples: | | | | | | |
| Total masses: | 23.511 g | | 27.385 g | | 29.967 g | |
| Sample Avg. | 2.351 g | | 2.489 g | | 2.497 g | |
| Loss %: | 32.6% | | 21.1% | | 14.1% | |
| Composition | Outer layer | | Outer layer | | Outer layer | |
| Xylitol | 28.570 g | Made 5 | | | 28.561 g | Made 6 |
| | | coatings | | | | coatings |
| | | of inner | | | | of inner |
| | | cores | | | | cores |
| Kollidon CL | 5.714 g | Masses: | | | 5.721 g | Masses: |
| Licorice Extract | 0.012 g | 1-7.182 g | | | 0.0125 g | 1-7.393 g |
| Glycerin | 7 drops | 2-6.753 g | | | 7 drops | 2-6.523 g |
| Green Tea Extract | 0.013 g | 3-6.825 g | | | 0.0125 g | 3-5.707 g |
| Eucalyptus oil | 3 drops | 4-6.876 g | | | 3 drops | 4-6.206 g |
| Arginine | 0.354 g | 5-6.791 g | | | 0.352 g | 5-6.290 g |
| Tutti-Frutti | 6 drops | | | | 0.00 g | 6-6.468 g |

[symbol]

indicates data missing or illegible when filed

The results of the fluoride and calcium release are summarized in Table 51.

TABLE 51

| | |
|---|---|
| 1-1 | Average fluoride release of fluoride containing lozenges is 7-8 mg/L |
| | NaF/Lozenge = 1.6 mg |
| | Fluoride/Lozenge = 0.452 × 1.6 = 0.723 mg |
| | 0.723 mg . . . 60 ml DIW |
| | (0.723 1000)/60 = 12.05 mg |
| 1-2 | Average calcium release of fluoride containing lozenges is 2.4 mg/L |
| | CaCl₂/lozenge = 1.5 mg |
| | Calcium/lozenge = 0.361 × 1.5 = 0.541 mg |
| | 0.541 mg . . . 60 ml DIW |
| | (0.541 × 1000)/60 = 9 mg |
| 2- | Average calcium release of fluoride free lozenges is 2.3 mg/L |
| | CaCl₂/lozenge = 1.5 mg |
| | Calcium/lozenge = 0.361 × 1.5 = 0.541 mg |
| | 0.541 mg . . . 60 ml DIW |
| | (0.541 × 1000)/60 = 9 mg |
| 3- | Average calcium release of Organic Matcha lozenges is 2 mg/L |
| | CaCl₂/lozenge = 1.5 mg |

### Example 28: Hardness Recovery after Treatment with Tea Made from Matcha Powder

In this example, we studied the hardness recovery of enamel softened by hydrochloric acid and remineralized in saliva or artificial saliva after rinsing with tea made from Matcha powder.

### Method

The effect of acid reflux on enamel was mimicked by immersing 10 polished extracted human molar in 5 mL of 0.01 M hydrochloric acid having a pH 2.5 for 10 minutes followed by rinsing with 5 mL of deionized water (DI), dried with compressed air. The baseline hardness (VHNbₐₛₑₗᵢₙₑ) of the 10 polished extracted human molars was measured. The hardness of the molars after the softening with hydrochloric acid was also measured to provide (VHN_{soft}).

A double concentration of artificial saliva was prepared according to table 52:

TABLE 52

To make 100 mL artificial saliva, combine the following:
100 ml deionized water
0.0221 g 1.5 mM CaCl₂•2H₂O
0.0122 g 0.9 mM KH₂PO₄
0.4766 g 20 mM HEPES buffer
0.9693 g 130 mM KCl
few drops 1M KOH to adjust to pH 7.0
Formula modified from: Mukai Y, Lagerweij MD, ten Cate JM. Effect of a solution with high fluoride concentration on re-mineralization of shallow and deep root surface caries in vitro. Caries Res 2001; 35: 317- 324.

A double concentration of Matcha green tea was also prepared by dissolving 2.7 g of Matcha-Love powder in 30 mL DI water. Remineralization or rehardening stage was conducted on a control and test groups of five samples.

In the control group the enamel samples in 5 ml were immersed in artificial saliva (dilute the double concentrated artificial saliva with DI water) for 1 hour.

In the test group, the Matcha green tea test solution was 15 ml double concentrated Matcha green tea with 15 mL double concentrated artificial saliva. The enamel samples were immersed in 5 ml Matcha green tea test solution for 1 hour. The resulting measured hardness of the rehardening (reminineralizing) stage was designated as (VHNᵣₑₘᵢₙ) The calculated % hardness recovery using formula % hardness recovery=100×(VHNᵣₑₘᵢₙ−VHN_{soft})/(VHN_{baseline}−VHN_{soft}). The results of this study are summarized in Table 53. Based on the findings summarized in Table 53 it was apparent the Matcha-Love rehardening effect was 23.3% higher than the control group (artificial saliva or saliva-like solution), which was 7.6%.

TABLE 53
Matcha Love tea in saliva-like solution (December 2019)

| Samples | Baseline | Avg. | Softening | Avg. | Remin. | Avg. | | % Recovery |
|---|---|---|---|---|---|---|---|---|
| 1B | 371.3 | | 317 | | 336.4 | | | |
| | 371.3 | 365.1 | 295.5 | 300.5 | 300.3 | 317.3 | | 26.0 |
| | 352.7 | | 288.9 | | 315.2 | | | |
| 3B | 361.5 | | 283.9 | | 321.5 | | | |
| | 331.6 | 343.0 | 289.7 | 284.4 | 324.3 | 322.9 | | 65.7 |
| | 335.9 | | 279.5 | | 322.9 | | | |
| 4B | 343.1 | | 278.1 | | 298.4 | | | |
| | 347.6 | 352.4 | 271.4 | 277.4 | 303.3 | 301.0 | | 31.4 |
| | 366.4 | | 282.8 | | 301.2 | | | |
| 5B | 367.8 | | 272.7 | | 301.4 | | | |
| | 385.2 | 375.9 | 277 | 281.1 | 292.8 | 300.2 | | 20.2 |
| 6B | 374.6 | | 293.6 | | 306.4 | | | |
| | 348.4 | | 293.2 | | 298.8 | | | |
| | 350.8 | 333.7 | 286.3 | 284.7 | 278.9 | 279.8 | | −10.1 |
| | 302 | | 274.6 | | 261.6 | | | |
| Matcha Love tea in salvia-like solution (December 2019) | | | | | | | Mean | 23.3 |
| | | | | | | | SD | 21.2 |

Saliva-like solution (December 2019)

| Samples | Baseline | Avg. | Softening | Avg. | Remin. | Avg. | | % Recovery |
|---|---|---|---|---|---|---|---|---|
| 1A | 333.9 | | 286.9 | | 300.3 | | | |
| | 379.7 | 350.2 | 309.5 | 293.2 | 279.3 | 283.9 | | −16.3 |
| 3A | 336.9 | | 283.2 | | 272.1 | | | |
| | 373.7 | | 341.3 | | 307.7 | | | |
| | 402.2 | 383.3 | 311.1 | 326.9 | 346.2 | 332.3 | | 9.7 |
| | 374 | | 328.2 | | 343.1 | | | |
| 4A | 334.6 | | 263.3 | | 267.4 | | | |
| | 353.5 | 347.1 | 257.5 | 263.9 | 268.3 | 268.9 | | 6.0 |
| | 353.2 | | 270.9 | | 271 | | | |
| 5A | 389.6 | | 290.1 | | 289.9 | | | |
| | 364.1 | 359.7 | 298.4 | 298.2 | 279.5 | 287.1 | | −17.9 |
| 6A | 325.5 | | 306 | | 292 | | | |
| | 362.7 | | 299.5 | | 322.2 | | | |
| | 360.4 | 367.9 | 307.3 | 305.1 | 331.6 | 325.2 | | 32.0 |
| | 380.6 | | 308.6 | | 321.9 | | | |
| Saliva-like solution (December 2019) | | | | | | | Mean | 7.6 |
| | | | | | | | SD | 18.5 |

### Example 29: Rehardening Effect of Fluoride-Free and Organic Matcha Inner Cores

In this example, we studied the hardness recovery of enamel softened by hydrochloric acid and remineralized in deionized water containing fluoride free inner core which, however contained organic Matcha green tea.

### Method

20 polished enamel samples from extracted human molars were prepared and the baseline hardness (VHNbaseline) was measured.

To establish acid erosion, each enamel sample was immersed in in 5 ml of 0.01 M hydrochloric acid (HCl) at pH 2.5 for 10 minutes, rinsed with 5 ml deionized (DI) water and dried with compressed air. The hardness of the softening stage (VHNsoft) was measured.

10 Matcha inner cores and 10 fluoride-free inner cores were prepared and each inner core was dissolved in 60 ml DI water.

Remineralization (rehardening stage) was conducted on 10 samples. In the fluoride-free inner core group, the enamel samples were immersed in 5 mL solution for 1 hour. In the Matcha inner core group the enamel samples were immersed in 5 mL solution for 1 hour. The hardness of the rehardening (remin) stage (VHNremin) was measured. The calculated % hardness recovery using formula % hardness recovery=100×(VHNremin−VHNsoft)/(VHNbaseline−VHNsoft).

In addition, pH of the solutions were measured for the first 5 samples in each group. The pH of the solutions from fluoride-free inner core group were 7.2, 7.5, 7.2, 7.3, 7.5. The pH of the solutions from Matcha inner core group were 7, 6.6, 6.8, 7, 7

The results of this study are summarized in Table 54. Based on the findings summarized in Table 54, it was evident that organic Matcha inner core group had 18.6% rehardening effect, which was higher than 12.6% rehardening by the fluoride-free inner core group.

TABLE 54
Fluoride-free Inner Cores with Ca & Phosphate):

| Samples | Baseline | Avg. | Softening | Avg. | Remin. | Avg. | | % Recovery |
|---|---|---|---|---|---|---|---|---|
| 9A | 340 | | 289.5 | | 298.2 | | | |
| | 363.8 | 350.8 | 302.5 | 300.2 | 290.5 | 303.5 | | 6.5 |
| | 348.6 | | 308.6 | | 321.7 | | | |
| 10A | 343.9 | | 300.1 | | 315.4 | | | |
| | 355.4 | 348.6 | 306.6 | 301.5 | 317.3 | 319.8 | | 38.9 |
| | 346.5 | | 297.8 | | 326.7 | | | |
| 12A | 331.9 | | 280 | | 295.3 | | | |
| | 347.3 | 343.4 | 301 | 295.4 | 288.3 | 288.0 | | −15.3 |
| | 351.1 | | 305.1 | | 280.4 | | | |
| 13A | 354.3 | | 311.5 | | 314 | | | |
| | 365.5 | 355.2 | 297.1 | 301.1 | 312.9 | 305.8 | | 8.6 |
| | 345 .7 | | 294.8 | | 290.5 | | | |
| 14A | 344.1 | | 291.6 | | 288.5 | | | |
| | 339.2 | 346.1 | 291.6 | 286.5 | 283.2 | 286.1 | | −0.8 |
| | 354.9 | | 276.4 | | 286.5 | | | |
| 7A | 337.2 | | 276.6 | | 258.3 | | | |
| | 327.7 | 331.8 | 265.4 | 265.9 | 261.2 | 262.7 | | −4.9 |
| | 330.4 | | 255.8 | | 268.7 | | | |
| 2A | 331.1 | | 232.8 | | 240.8 | | | |
| | 337.7 | 330.3 | 238.3 | 234.9 | 253.1 | 245.8 | | 11.5 |
| | 322.2 | | 233.5 | | 243.5 | | | |
| 52B | 324.8 | | 299.9 | | 306.6 | | | |
| | 329.4 | 333.0 | 285.1 | 287.3 | 298.2 | 306.8 | | 42.7 |
| | 344.7 | | 276.8 | | 315.6 | | | |
| 7B | 335.9 | | 279.7 | | 292.6 | | | |
| | 334.9 | 335.2 | 294.2 | 287.5 | 292.2 | 295.5 | | 16.7 |
| | 334.9 | | 288.5 | | 301.6 | | | |
| 5B | 344.9 | | 279.1 | | 299.3 | | | |
| | 330.4 | 350.4 | 315 | 298.0 | 327.7 | 309.5 | | 22.0 |
| | 375.8 | | 299.9 | | 301.6 | | | |
| Fluoride-free Inner Cores (with Co & Phosphate): | | | | | | | Mean | 12.6 |
| | | | | | | | SD | 18.3 |

### Example 30: Rehardening Effect of Organic Matcha Inner Cores with Tripled Calcium Phosphate

In this example, we studied the hardening effect on of enamel softened by hydrochloric acid and remineralized in deionized water containing organic Matcha inner cores with tripled calcium phosphate. We found that Matcha green tea had 24% rehardening effect, but when more Matcha powder was added to the inner core, the core was not ‘workable’. Moreover, adding more Matcha green tea would result in increased fluoride concentration which can be negative to bone health. However, calcium and phosphate content could be safely increased. Thus, Matcha inner core with triple amount of calcium, phosphate, and casein was developed. Table 55 below is a graph of organic Matcha green tea inner cores having a normal concentration of Matcha.

TABLE 55
Organic Matcha Inner Cores (Normal Ca & Phosphate):

| Samples | Baseline | Avg. | Softening | Avg. | Remin. | Avg. | | % Recovery |
|---|---|---|---|---|---|---|---|---|
| 9B | 337.9 | | 280.2 | | 301.4 | | | |
| | 335.1 | 338.8 | 292.2 | 283.1 | 293.8 | 295.6 | | 22.5 |
| | 343.4 | | 276.8 | | 291.6 | | | |
| 10B | 335.9 | | 250.6 | | 285.3 | | | |
| | 315.4 | 333.2 | 271.2 | 260.1 | 291.1 | 288.8 | | 39.3 |
| | 348.4 | | 258.5 | | 290.1 | | | |
| 12B | 367.3 | | 284.1 | | 296.9 | | | |
| | 366.7 | 368.8 | 289.9 | 293.5 | 294.2 | 293.8 | | 0.4 |
| | 372.5 | | 306.4 | | 290.3 | | | |
| 13B | 290.3 | | 226.2 | | 227.2 | | | |
| | 294.4 | 300.5 | 258.3 | 238.0 | 230.5 | 232.1 | | −9.4 |
| | 316.8 | | 229.6 | | 238.7 | | | |
| 14B | 348.1 | | 281.2 | | 283.2 | | | |
| | 369.6 | 360.3 | 266.2 | 274.6 | 281.2 | 280.2 | | 6.5 |
| | 363.2 | | 276.4 | | 276.1 | | | |
| 10B* | 337.2 | | 303.3 | | 313.3 | | | |
| | 333.1 | 336.3 | 295.3 | 302.3 | 307.7 | 308.6 | | 18.5 |
| | 338.7 | | 308.4 | | 304.9 | | | |
| 6B | 345.7 | | 274.2 | | 269.4 | | | |
| | 337.2 | 338.4 | 267.4 | 280.1 | 304.6 | 291.4 | | 19.4 |
| | 332.4 | | 298.8 | | 300.3 | | | |
| 12B* | 344.1 | | 270.5 | | 270.7 | | | |
| | 335.6 | 334.8 | 264.7 | 261.8 | 277 | 271.4 | | 13.1 |
| | 324.6 | | 250.3 | | 266.5 | | | |
| 11B | 354.6 | | 318.4 | | 310.4 | | | |
| | 365.2 | 361.0 | 271.8 | 301.0 | 305.1 | 308.9 | | 13.3 |
| | 363.2 | | 312.7 | | 311.3 | | | |
| 9B* | 322.4 | | 275.9 | | 309.7 | | | |
| | 348.4 | 336.5 | 281.8 | 276.4 | 305.7 | 313.6 | | 61.9 |
| | 338.7 | | 271.6 | | 325.5 | | | |
| Organic Matcha Inner Cores (Normal Ca & Phosphate): | | | | | | | Mean | 18.6 |
| | | | | | | | SD | 20.1 |

### Method

The methodology for preparing Matcha green tea inner cores with tripled calcium and phosphate included preparing 10 polished enamel samples from extracted human molars having a baseline hardness (VHN_{baseline}). To establish acid erosion each enamel sample was immersed in 5 ml of 0.01 M hydrochloric acid (HCl) at pH 2.5 for 10 minutes, rinsed with 5 ml deionized (DI) water and dried with compressed air. The hardness of the softening stage (VHNsoft) was measured. 10 Matcha inner cores with tripled calcium and phosphate were prepared and each inner core was dissolved in 60 ml DI water. Remineralization (rehardening stage) was conducted by immersing enamel samples in 5 ml solution for 1 hour. The hardness of the rehardening (remin) stage (VHNremin) was measured. % hardness recovery was calculated by using formula: % hardness recovery=100×(VHNᵣₑₘᵢₙ−VHN_{soft})/(VHN_{baseline}−VHN_{soft}).

The results of this study are summarized in Table 56. From this study, it became apparent that when the concentration of calcium and phosphate was tripled in organic Matcha inner cores, the rehardening effect increased to 23%.

TABLE 56
Organic Matcha Inner Cores (Trilped Ca & Ph content):

| Samples | Baseline | Avg. | Softening | Avg. | Remin. | Avg. | | % Recovery |
|---|---|---|---|---|---|---|---|---|
| 1A | 346.8 | | 301 | | 295.3 | | | |
| | 337.4 | 339.9 | 271.6 | 282.6 | 292.2 | 282.9 | | 18.1 |
| | 335.4 | | 275.1 | | 291.3 | | | |
| 2A | 328 | | 298.2 | | 303.8 | | | |
| | 324.6 | 333.0 | 288.1 | 291.6 | 314.5 | 307.8 | | 39.1 |
| | 346.5 | | 288.5 | | 305.1 | | | |
| 3A | 328.9 | | 303.6 | | 306.4 | | | |
| | 318.2 | 327.5 | 292.8 | 297.2 | 314.5 | 311.7 | | 48.0 |
| | 335.4 | | 295.1 | | 314.3 | | | |
| 4A | 376.1 | | 293.6 | | 310.6 | | | |
| | 382.8 | 376.2 | 277 | 286.4 | 309.1 | 308.4 | | 24.5 |
| | 369.6 | | 288.5 | | 305.5 | | | |
| 5A | 332.6 | | 249.4 | | 297.4 | | | |
| | 335.4 | 330.9 | 256.6 | 261.5 | 307.3 | 295.3 | | 48.7 |
| | 324.8 | | 278.5 | | 281.2 | | | |
| 6A | 324.8 | | 281.6 | | 281.8 | | | |
| | 334.6 | 335.7 | 271 | 278.8 | 279.1 | 281.5 | | 4.6 |
| | 347.8 | | 283.9 | | 283.5 | | | |
| 7A | 319.4 | | 273.2 | | 280.2 | | | |
| | 331.4 | 332.3 | 270.5 | 275.6 | 284.1 | 281.9 | | 11.1 |
| | 346 | | 283.2 | | 281.4 | | | |
| 8A | 318.2 | | 248.1 | | 282.8 | | | |
| | 314.7 | 315.6 | 247.3 | 250.8 | 261.2 | 275.0 | | 37.3 |
| | 314 | | 257.1 | | 281 | | | |
| 11A | 329.7 | | 283.7 | | 297.4 | | | |
| | 363.8 | 347.5 | 295.9 | 294.3 | 294 | 297.6 | | 6.2 |
| | 348.9 | | 303.3 | | 301.4 | | | |
| 12A | 352.7 | | 302.9 | | 299.9 | | | |
| | 349.2 | 356.0 | 303.8 | 305.2 | 316.3 | 306.9 | | 3.3 |
| | 366.1 | | 308.8 | | 304.4 | | | |
| Organic Matcha Inner Cores (Trilped Ca & Ph content): | | | | | | | Mean | 24.1 |
| | | | | | | | SD | 18.0 |

### Example 31: Comparison of Fluoride Levels in Commercially Available Black Tea, Green Tea, and Matcha Green Tea

In this study, we compared fluoride levels in commercially available black tea, green tea, and Matcha green tea.

### Materials and Methods

Varieties of tea were purchased from a local supermarket (Kroger) in Tennessee. These teas included three black teas (Bigelow© Earl Grey Black Tea, Twinnings© Lady Grey Black Tea, Lipton© Black Tea), two green teas (Bigelow© Green Tea Classic, Lipton© Green Tea), and three Matcha green teas (Mighty Leaf© Matcha, Celestial© Matcha green tea Bag, 2 containers of Matcha Love©). Tea samples were prepared according to the manufacturer's directions to mimic consumer activity and steeped for two minutes. Tea bags were brewed in 250 ml of just-boiling deionized water (DIW) and stirred for 2 minutes. Exceptions were Mighty Leaf Matcha© bag which was brewed in 300 mL DIW, and Matcha Love© Green Tea Powder of which 0.5 teaspoon of the tea powder (approximately 1 gram) was stirred for 2 minutes in 30 mL just-boiling DIW (manufacturer's instruction: 1 teaspoon in 2-3 oz. water; 2 oz. water is 60 ml). Deionized water was used as a negative control.

10 mL aliquots from each tea sample were combined with 10 mL Total Ionic Strength Adjustment Buffer (TISAB II, Mettler Toledo). Fluoride concentration was measured with a combination fluoride electrode (perfectION™, Mettler Toledo) coupled to a pH/ISE meter (S220 SevenCompact™ pH/Ion meter, Mettler Toledo). Fluoride ISE Standard 1000 mg/L (Mettler Toledo) was serially diluted to 0.1 mg/L for calibration. Fluoride concentration was calculated from the calibration curve. Each sample size was 5 per group. Results were analyzed using ANOVA followed by Student-Newman-Keuls post hoc tests at significance level 0.05.

### Results

Fluoride concentrations are shown in Table 1. Deionized water, the negative control, had negligible amount of fluoride (0.02 mg/L). Commercially available teas contained varying fluoride concentrations ranging from about 0.5 to about 6.1 mg/L, significantly different between brands and types of tea. Matcha Love© Green Tea Powder had the highest concentration of fluoride among the tested teas.

TABLE 57

| | Fluoride Concentration |
|---|---|
| Groups | (mg/L or ppm, mean ± SD) |
| Deionized water | 0.02 ± −0.003 ^{a} |
| Mighty Leaf © Matcha | 0.521 ± 0.1 ^{b} |
| Celestial © Matcha green tea Bag | 0.969 ± 0.085 ^{c} |
| Bigelow © Earl Grey Black Tea | 1.108 ± 0.025 ^{d} |
| Bigelow © Green Tea Classic | 1.296 ± 0.077 ^{e} |
| Lipton © Green Tea | 1.328 ± 0.069 ^{e} |
| Twinnings © Lady Grey Black Tea | 2.25 ± 0.063 ^{f} |
| Lipton © Black Tea | 2.534 ± 0.128 ^{g} |
| Matcha Love © No. 2 | 4.528 ± 0.058 ^{h} |
| Matcha Love © No. 1 | 6.082 ± 0.219 ⁱ |

Same superscript letters denote values that were not significantly different (ANOVA followed by Student-Newman-Keuls post hoc tests at significance level 0.05).

### Discussion

The health benefits of tea are well documented against cardiovascular diseases and certain types of cancer.²⁻⁷ For dental health the benefits of tea have less emphasis. Dental caries and tooth erosion are prevalent in the American population, partly due to the increase in consumption of acidic foods and drinks. Twenty percent of American teenagers drink at least two sugary drinks a day, and seven percent drink three or more. Some of the ‘healthier’ drink options, such as juices and fermented tea, also contain a large amount of sugar, nearly nine teaspoons of sugar per 20-ounce bottle, in addition to high acidic content.²⁰ The American Dental Association has warned against drinking fermented teas such as Kombacha due to its high acidic content.²¹ Recurrent exposure to acidic mediums reduce the pH in the oral cavity, and give rise to an increase in dental caries and tooth erosion. Could regular consumption of black, green, or Matcha green tea offer a healthy alternative to the sugary drinks? Our study showed that all tea samples have some amount of fluoride and thus could be a more tooth-friendly alternative to commercially available sugary drinks.

Although a minimal nutritional requirement of fluoride is not available, the US Public Health Service recommends 0.7 mg/L for effective community water fluoridation for caries prevention with limited risk of dental fluorosis.²³ In our study, we brewed tea following manufacturers' instructions, most of which suggested 250 ml of hot water (about a cup) per tea bag. Therefore, the calculated fluoride concentrations shown in Table 1 can be viewed as mg of fluoride intake per 4 cups (equivalent to 1000 ml or 1 L). For example, 4 cups of Lipton Black Tea provide 2.534 mg of fluoride. Tea made from Matcha green tea powder, despite having a higher fluoride concentration, is usually served in smaller amounts (60-90 ml). Thus 1 L of tea prepared from Matcha green tea powder, more than 10 servings, provides 4.528-6.082 mg fluoride.

The tea samples in this study simulated how a consumer would brew their tea according to the manufacturers. All except Matcha Love tea were contained in bags. The differences in fluoride content between the tested tea brands were mostly statistically significant. However, we did not see a pattern in fluoride content between black and green teas. Both black and green teas consist of leaves of Camellia sinensis plants. Black tea is fully oxidized (fermented) for taste characteristics, whereas green tea is derived from drying and steaming fresh tea leaves after harvesting.⁸ Fluoride concentrations of the tested green and black teas were in the same range as those reported in the literature, considering brewing time and amount of water used.^{18,22,25} All bagged teas were brewed for 2 minutes in this study. Longer brewing times increase fluoride concentrations.²⁵ The highest fluoride concentration was in tea made from Matcha Love powder (Table 1). Matcha, by definition, is a finely ground powder of green tea leaves. It is possible that tea made from Matcha Love powder had higher fluoride content because it required less water and all powder was left to dissolve, unlike tea brewed from tea bags, where bags were retrieved after 2 minutes. Interestingly, two containers of Matcha Love tea powder purchased at different days from the same supermarket had significant different fluoride content. Apparently, the fluoride content in Matcha green tea powder was not consistent between batches, which may be attributed to natural products or variation in the fineness of the ground.

In summary, brewed tea can be a source of fluoride and an effective means for reducing caries and demineralization of tooth enamel. The information about fluoride content in tea is important for dental health care professionals for its effectiveness.

### Conclusions

Supermarket teas contain significant levels of fluoride, mostly higher than 0.7 mg/L (ppm), the optimally recommended community water fluoridation. Matcha green tea powder had the highest fluoride content among the tested products. Tea is a potential natural fluoride source for caries prevention but may increase the risk of fluorosis when consumed excessively.

### Example 32

Testing Calcium release of Organic Matcha inner core (10× Ca & Ph):

Organic Matcha inner cores with 10× calcium (Ca) and phosphate (Ph) content in comparison to original inner core's composition.

Inner cores were made.

Samples #1, 3 & 6 are from the same batch.

Samples #2 & 7 are from the same batch.

Reading at 0 min background deionized (DI) water only.

| Time | Sample 1 | Sample 2 | Sample 3 | Sample 6 | Sample 7 | AVERAGE | STDEV |
|---|---|---|---|---|---|---|---|
| 0 min | 0.2 | 0.3 | 0.2 | 0.4 | 0.4 | 0 | 0 |
| 5 min | 3.5 | 3.6 | 6.7 | 6.9 | 6.9 | 6 | 2 |
| 10 min | 10.3 | 6.2 | 11.1 | 12.6 | 13 | 11 | 3 |
| 15 min | 15.1 | 10.9 | 18.5 | 18.5 | 13.2 | 15 | 3 |
| 20 min | 15.5 | 14 | 20.3 | 22.1 | 16.8 | 18 | 3 |
| 25 min | 18.4 | 14.5 | 25.2 | 22.1 | 21.2 | 20 | 4 |
| 30 min | 17.7 | 16.8 | 27.3 | 27.6 | 24 | 23 | 5 |
| 35 min | 19.9 | 20.6 | 27.9 | 33 | 27.2 | 26 | 5 |
| 40 min | 21.8 | 19.6 | 29.8 | 33.2 | 27.1 | 26 | 6 |
| 45 min | 24 | 20.8 | 32 | 36.8 | 28.3 | 28 | 6 |
| 50 min | 25.3 | 22.2 | 32.8 | 33.3 | 29.9 | 29 | 5 |
| 55 min | 25.7 | 21.8 | 33.9 | 36.9 | 30.5 | 30 | 6 |
| 60 min | 26.2 | 22.6 | 32.1 | 34.4 | 29.5 | 29 | 5 |
| 65 min | 26.5 | 19.9 | 33.5 | 36.9 | 29.2 | 29 | 7 |
| 120 min | 20.8 | 22.3 | 33.9 | 34.1 | 28.3 | 28 | 6 |

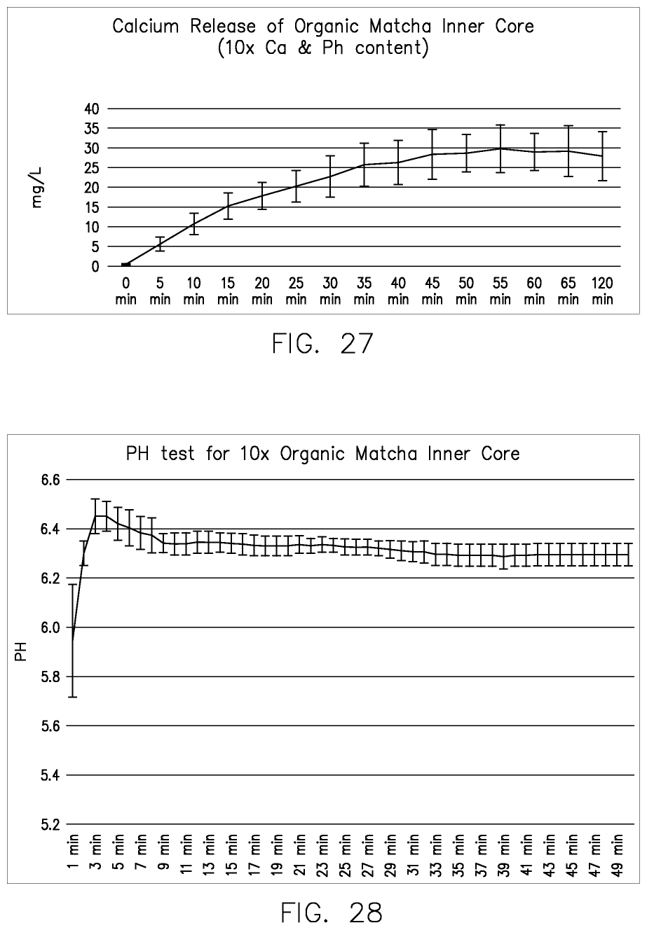

The results are shown in the graph in FIG. 27.

### Example 33

PH test for 10× Organic Matcha Inner Cores:

Organic Matcha inner core were prepared.

Sample #1 is from batch no. 1.

Samples #2 & 3 are from batch no. 2.

| Time | | Sample 1 | Sample 2 | Sample 3 | AVERAGE | STDEV |
|---|---|---|---|---|---|---|
| 1 | min | 5.99 | 6.15 | 5.7 | 5.9 | 0.23 |
| 2 | min | 6.31 | 6.35 | 6.25 | 6.3 | 0.05 |
| 3 | min | 6.39 | 6.44 | 6.53 | 6.5 | 0.07 |
| 4 | min | 6.4 | 6.44 | 6.52 | 6.5 | 0.06 |
| 5 | min | 6.38 | 6.39 | 6.5 | 6.4 | 0.07 |
| 6 | min | 6.35 | 6.38 | 6.49 | 6.4 | 0.07 |
| 7 | min | 6.33 | 6.37 | 6.46 | 6.4 | 0.07 |
| 8 | min | 6.31 | 6.37 | 6.45 | 6.4 | 0.07 |
| 9 | min | 6.3 | 6.37 | 6.36 | 6.3 | 0.04 |
| 10 | min | 6.29 | 6.38 | 6.35 | 6.3 | 0.05 |
| 11 | min | 6.29 | 6.38 | 6.35 | 6.3 | 0.05 |
| 12 | min | 6.3 | 6.39 | 6.35 | 6.3 | 0.05 |
| 13 | min | 6.3 | 6.39 | 6.35 | 6.3 | 0.05 |
| 14 | min | 6.31 | 6.39 | 6.34 | 6.3 | 0.04 |
| 15 | min | 6.32 | 6.39 | 6.32 | 6.3 | 0.04 |
| 16 | min | 6.32 | 6.39 | 6.31 | 6.3 | 0.04 |
| 17 | min | 6.32 | 6.38 | 6.3 | 6.3 | 0.04 |
| 18 | min | 6.34 | 6.37 | 6.29 | 6.3 | 0.04 |
| 19 | min | 6.34 | 6.37 | 6.29 | 6.3 | 0.04 |
| 20 | min | 6.34 | 6.37 | 6.29 | 6.3 | 0.04 |
| 21 | min | 6.34 | 6.37 | 6.3 | 6.3 | 0.04 |
| 22 | min | 6.34 | 6.36 | 6.3 | 6.3 | 0.03 |
| 23 | min | 6.35 | 6.36 | 6.3 | 6.3 | 0.03 |
| 24 | min | 6.35 | 6.35 | 6.3 | 6.3 | 0.03 |
| 25 | min | 6.35 | 6.34 | 6.29 | 6.3 | 0.03 |
| 26 | min | 6.35 | 6.34 | 6.29 | 6.3 | 0.03 |
| 27 | min | 6.35 | 6.34 | 6.29 | 6.3 | 0.03 |
| 28 | min | 6.35 | 6.33 | 6.29 | 6.3 | 0.03 |
| 29 | min | 6.35 | 6.32 | 6.28 | 6.3 | 0.04 |
| 30 | min | 6.35 | 6.32 | 6.27 | 6.3 | 0.04 |
| 31 | min | 6.35 | 6.31 | 6.27 | 6.3 | 0.04 |
| 32 | min | 6.35 | 6.31 | 6.26 | 6.3 | 0.05 |
| 33 | min | 6.34 | 6.3 | 6.25 | 6.3 | 0.05 |
| 34 | min | 6.34 | 6.3 | 6.25 | 6.3 | 0.05 |
| 35 | min | 6.34 | 6.29 | 6.25 | 6.3 | 0.05 |
| 36 | min | 6.34 | 6.29 | 6.25 | 6.3 | 0.05 |
| 37 | min | 6.34 | 6.29 | 6.25 | 6.3 | 0.05 |
| 38 | min | 6.34 | 6.29 | 6.25 | 6.3 | 0.05 |
| 39 | min | 6.34 | 6.29 | 6.24 | 6.3 | 0.05 |
| 40 | min | 6.34 | 6.29 | 6.25 | 6.3 | 0.05 |
| 41 | min | 6.34 | 6.29 | 6.25 | 6.3 | 0.05 |
| 42 | min | 6.34 | 6.3 | 6.25 | 6.3 | 0.05 |
| 43 | min | 6.34 | 6.3 | 6.25 | 6.3 | 0.05 |
| 44 | min | 6.34 | 6.3 | 6.25 | 6.3 | 0.05 |
| 45 | min | 6.34 | 6.3 | 6.25 | 6.3 | 0.05 |
| 46 | min | 6.34 | 6.3 | 6.25 | 6.3 | 0.05 |
| 47 | min | 6.34 | 6.3 | 6.25 | 6.3 | 0.05 |
| 48 | min | 6.34 | 6.3 | 6.25 | 6.3 | 0.05 |
| 49 | min | 6.34 | 6.3 | 6.25 | 6.3 | 0.05 |
| 50 | min | 6.34 | 6.3 | 6.25 | 6.3 | 0.05 |

The results are shown in the graph in FIG. 28.

### Example 34

Fluoride release test for 10× Organic Matcha inner core:

Inner cores were made.

Reading at 0 min Background DI water only

| Time | | Sample 1 | Sample 2 | Sample 3 | AVERAGE | STDEV |
|---|---|---|---|---|---|---|
| 0 | min | 0.05 | 0.05 | 0.05 | 0.05 | 0.00 |
| 5 | min | 0.09 | 0.06 | 0.09 | 0.08 | 0.02 |
| 10 | min | 0.09 | 0.08 | 0.1 | 0.09 | 0.01 |
| 15 | min | 0.1 | 0.1 | 0.1 | 0.10 | 0.00 |
| 20 | min | 0.12 | 0.11 | 0.1 | 0.11 | 0.01 |
| 25 | min | 0.13 | 0.14 | 0.11 | 0.13 | 0.02 |
| 30 | min | 0.14 | 0.15 | 0.1 | 0.13 | 0.03 |
| 35 | min | 0.15 | 0.15 | 0.13 | 0.14 | 0.01 |
| 40 | min | 0.16 | 0.16 | 0.14 | 0.15 | 0.01 |
| 45 | min | 0.16 | 0.16 | 0.14 | 0.15 | 0.01 |
| 50 | min | 0.16 | 0.16 | 0.15 | 0.16 | 0.01 |
| 55 | min | 0.17 | 0.16 | 0.16 | 0.16 | 0.01 |
| 60 | min | 0.17 | 0.17 | 0.17 | 0.17 | 0.00 |
| 65 | min | 0.18 | 0.16 | 0.17 | 0.17 | 0.01 |
| 70 | min | 0.18 | 0.16 | 0.17 | 0.17 | 0.01 |
| 120 | min | 0.18 | 0.16 | 0.16 | 0.17 | 0.01 |

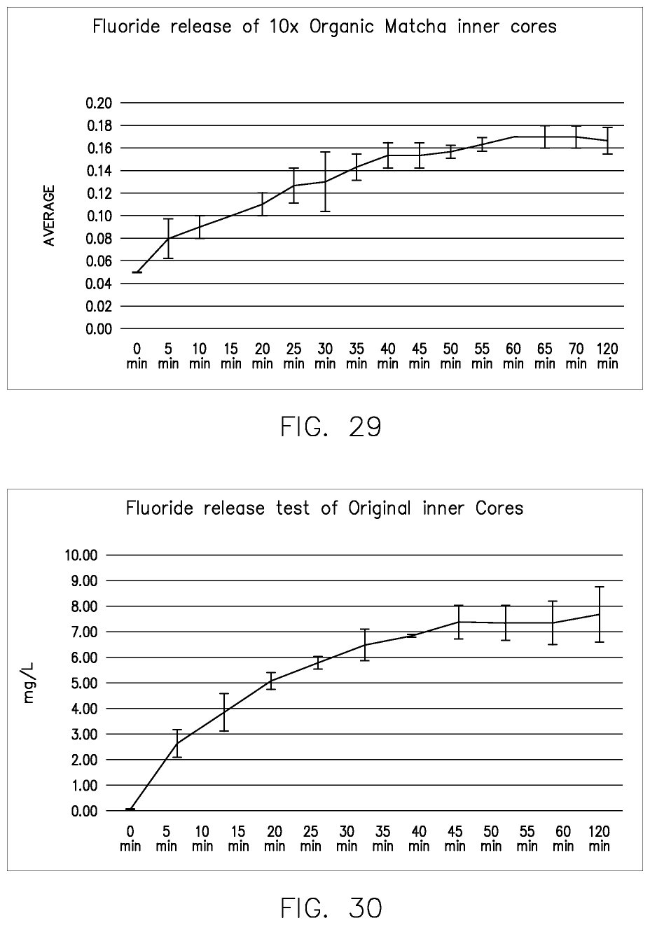

The results are shown in the graph in FIG. 29. The formulation used in this release test is listed in Table 49.

### Example 35

New original inner cores were made and they were tested for fluoride release, calcium release and PH. Fluoride release test for Original Inner cores:

Original inner cores were made.

Samples #1 & 3 are from batch no. 1

Sample #2 is from batch no. 2

Reading at 0 min Background DI water only.

| Time | | Sample 1 | Sample 2 | Sample 3 | AVERAGE | STDEV |
|---|---|---|---|---|---|---|
| 0 | min | 0.03 | 0.03 | 0.04 | 0.03 | 0.01 |
| 5 | min | 2.9 | 2 | 3 | 2.63 | 0.55 |
| 10 | min | 4.1 | 3 | 4.4 | 3.83 | 0.74 |
| 15 | min | 4.7 | 5.3 | 5.2 | 5.07 | 0.32 |
| 20 | min | 5.5 | 6 | 5.8 | 5.77 | 0.25 |
| 25 | min | 5.9 | 7.1 | 6.4 | 6.47 | 0.60 |
| 30 | min | 6.8 | 6.9 | 6.8 | 6.83 | 0.06 |
| 35 | min | 7.1 | 8.1 | 6.9 | 7.37 | 0.64 |
| 40 | min | 7 | 8.1 | 6.9 | 7.33 | 0.67 |
| 60 | min | 6.9 | 8.3 | 6.8 | 7.33 | 0.84 |
| 120 | min | 7.2 | 8.9 | 6.9 | 7.67 | 1.08 |

The results are shown in the graph in FIG. 30. The inner core formulation used in this release test is listed in Table 1.

### Example 36

Calcium release test for Original Inner cores of the lozenge:

Original inner cores were made.

Sample #1 is from batch no. 1

Samples #2 & 3 are from batch no. 2

Reading at 0 min Background DI water only.

| Time | | Sample 1 | Sample 2 | Sample 3 | AVERAGE | STDEV |
|---|---|---|---|---|---|---|
| 0 | min | 0.2 | 0.3 | 0.3 | 0.3 | 0.1 |
| 5 | min | 0.9 | 0.7 | 1.3 | 1.0 | 0.3 |
| 10 | min | 1 | 1 | 1.4 | 1.1 | 0.2 |
| 15 | min | 1.3 | 1.9 | 2.6 | 1.9 | 0.7 |
| 20 | min | 1.6 | 1.8 | 2.6 | 2.0 | 0.5 |
| 25 | min | 1.7 | 2.1 | 2.7 | 2.2 | 0.5 |
| 30 | min | 1.8 | 2 | 3.1 | 2.3 | 0.7 |
| 35 | min | 1.9 | 1.9 | 3.1 | 2.3 | 0.7 |
| 60 | min | 1.9 | 1.9 | 3.3 | 2.4 | 0.8 |
| 120 | min | 2.1 | 2 | 3.2 | 2.4 | 0.7 |

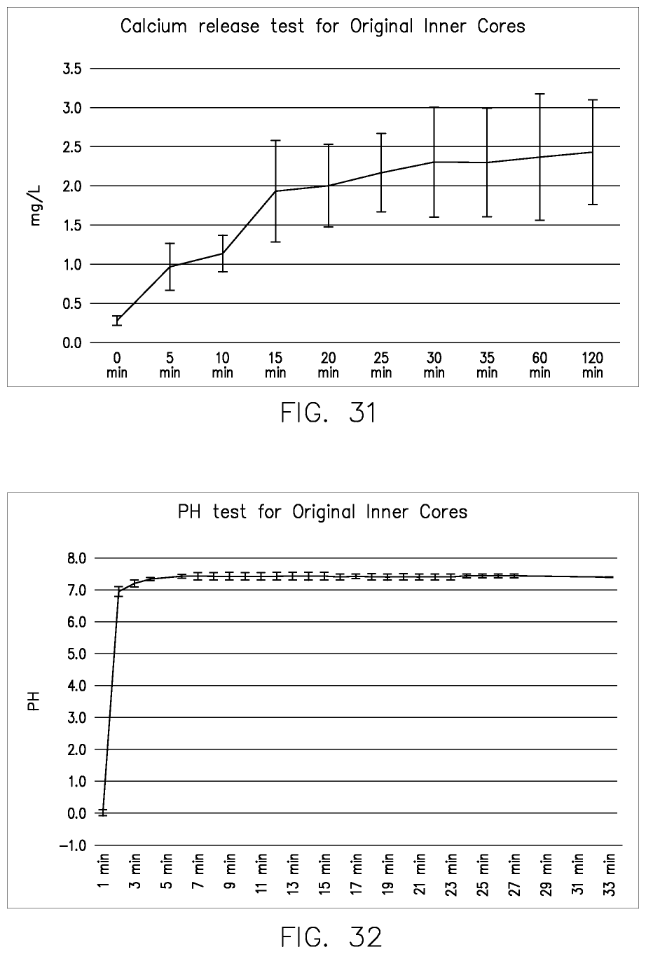

The results are shown in the graph in FIG. 31.

It was noted that the average Ca release from the Original inner core and MI paste after 30 min. is approximately 2.3 mg/L and 1.2 mg/L respectively, where the release from the Original inner core is approximately double compared to that of the MI paste. Specifically, MI Paste is a commercially available paste that uses calcium and phosphate to replenish minerals in teeth and strengthen them.

### Example 37

PH test for Original inner cores:

Original inner cores were made.

Samples #1 & 3 are from batch no. 1.

Sample #2 is from batch no. 2.

| Time | | Sample 1 | Sample 2 | sample 3 | AVERAGE | STDEV |
|---|---|---|---|---|---|---|
| 1 | min | 6.5 | 6.7 | 6.6 | 6.6 | 0.1 |
| 2 | min | 6.9 | 7.1 | 6.8 | 6.9 | 0.2 |
| 3 | min | 7.1 | 7.2 | 7.3 | 7.2 | 0.1 |
| 4 | min | 7.3 | 7.4 | 7.3 | 7.3 | 0.1 |
| 5 | min | 7.4 | 7.4 | 7.4 | 7.4 | 0.0 |
| 6 | min | 7.4 | 7.5 | 7.4 | 7.4 | 0.1 |
| 7 | min | 7.3 | 7.5 | 7.5 | 7.4 | 0.1 |
| 8 | min | 7.3 | 7.5 | 7.5 | 7.4 | 0.1 |
| 9 | min | 7.3 | 7.5 | 7.5 | 7.4 | 0.1 |
| 10 | min | 7.3 | 7.5 | 7.5 | 7.4 | 0.1 |
| 11 | min | 7.3 | 7.5 | 7.5 | 7.4 | 0.1 |
| 12 | min | 7.3 | 7.5 | 7.5 | 7.4 | 0.1 |
| 13 | min | 7.3 | 7.5 | 7.5 | 7.4 | 0.1 |
| 14 | min | 7.3 | 7.5 | 7.5 | 7.4 | 0.1 |
| 15 | min | 7.3 | 7.5 | 7.5 | 7.4 | 0.1 |
| 16 | min | 7.3 | 7.4 | 7.5 | 7.4 | 0.1 |
| 17 | min | 7.4 | 7.4 | 7.5 | 7.4 | 0.1 |
| 18 | min | 7.3 | 7.4 | 7.5 | 7.4 | 0.1 |
| 19 | min | 7.3 | 7.4 | 7.5 | 7.4 | 0.1 |
| 20 | min | 7.3 | 7.4 | 7.5 | 7.4 | 0.1 |
| 21 | min | 7.3 | 7.4 | 7.5 | 7.4 | 0.1 |
| 22 | min | 7.3 | 7.4 | 7.5 | 7.4 | 0.1 |
| 23 | min | 7.3 | 7.4 | 7.5 | 7.4 | 0.1 |
| 24 | min | 7.4 | 7.4 | 7.5 | 7.4 | 0.1 |
| 25 | min | 7.4 | 7.4 | 7.5 | 7.4 | 0.1 |
| 26 | min | 7.4 | 7.4 | 7.5 | 7.4 | 0.1 |
| 27 | min | 7.4 | 7.4 | 7.5 | 7.4 | 0.1 |
| 28 | min | 7.4 | 7.4 | 7.4 | 7.4 | 0.0 |
| 29 | min | 7.4 | 7.4 | 7.4 | 7.4 | 0.0 |
| 30 | min | 7.4 | 7.4 | 7.4 | 7.4 | 0.0 |
| 31 | min | 7.4 | 7.4 | 7.4 | 7.4 | 0.0 |
| 32 | min | 7.4 | 7.4 | 7.4 | 7.4 | 0.0 |
| 33 | min | 7.4 | 7.4 | 7.4 | 7.4 | 0.0 |

The results are shown in the graph in FIG. 32

### Example 38

Measure particle size of ground Organic Matcha powder:

Grinding procedures:

Weigh 10 g of Organic Matcha powder.

Repeat the previous step 8 times and put each 10 g in a separate container.

Start the grinding process by grinding 10 g of Matcha at a time for 15 minutes using a mortar and pestle.

After grinding, add the 8 groups all together in one container.

Shake the ground Matcha powder thoroughly for 30 minutes.

Take 2 g of ground Matcha powder and test its Fluoride release as follows:

1. Put 2 g of refined Matcha powder in 60 ml DI water.

2. Adjust the stir plate speed at 250 RPM at room temperature.

3. Take 1 ml sample every 5 min for 30 min.

4. Add 1 ml TISAB II and measure F release of each sample.

5. Add 1 ml DI water every 5 min to the glass beaker to keep the 60 ml soln. constant.

Data:

| | |
|---|---|
| Sample #1 | Sample #2 |

| | | | | |
|---|---|---|---|---|
| 0 min. | 0.08 | mg/L | 0.06 | mg/L |
| 5 min. | 1.7 | mg/L | 1.5 | mg/L |
| 10 min. | 1.9 | mg/L | 2 | mg/L |
| 15 min. | 2 | mg/L | 1.9 | mg/L |
| 20 min. | 2 | mg/L | 2 | mg/L |
| 25 min. | 2 | mg/L | 2 | mg/L |
| 30 min. | 2 | mg/L | 2 | mg/L |

5 g of ground Organic Matcha powder taken and grinded again for 15 minutes then, F release test done using 2 g following the previous procedures:

Data:

| | |
|---|---|
| 0 min. | 0.07 mg/L |
| 5 mn. | 1.2 mg/L |
| 10 min. | 1.5 mg/L |
| 15 min. | 1.8 mg/L |
| 20 min. | 1.8 mg/L |
| 25 min. | 1.8 mg/L |
| 30 min. | 1.8 mg/L |

Observation: After dissolving Matcha powder in DI water, its color was dark green. In other words, double grinded Matcha has higher Chroma than one time grinded Matcha.

### Example 39

Fluoride release test for MI Paste Plus in DI water:

Steps:

1. Weigh 0.12 g of MI paste plus on a weighing paper.

2. Put 60 ml DI water in a 250 ml glass beaker.

3. Adjust stir plate speed at 250 RPM at room temperature.

4. Put a stir bar in the middle of the glass beaker.

5. Add 0.12 g of MI paste plus to the 60 ml DI water.

6. Take measurements every 5 minutes by taking 1 ml sample and add 1 ml TISAB II to it.

7. Add 1 ml DI water to the glass beaker every 5 minutes to keep the 60 ml soln. constant

| Time | Sample 1 | Sample 2 | Sample 3 | AVERAGE | STDEV |
|---|---|---|---|---|---|
| 0 min | 0.1 | 0.06 | 0.07 | 0.1 | 0.02 |
| 5 min | 1.8 | 1.2 | 1 | 1.3 | 0.42 |
| 10 min | 1.9 | 1.9 | 1.7 | 1.8 | 0.12 |
| 15 min | 1.9 | 1.9 | 1.8 | 1.9 | 0.06 |
| 20 min | 1.9 | 1.8 | 1.7 | 1.8 | 0.10 |
| 25 min | 1.9 | 1.8 | 1.7 | 1.8 | 0.10 |

Reading at 0 min Background DI water only.

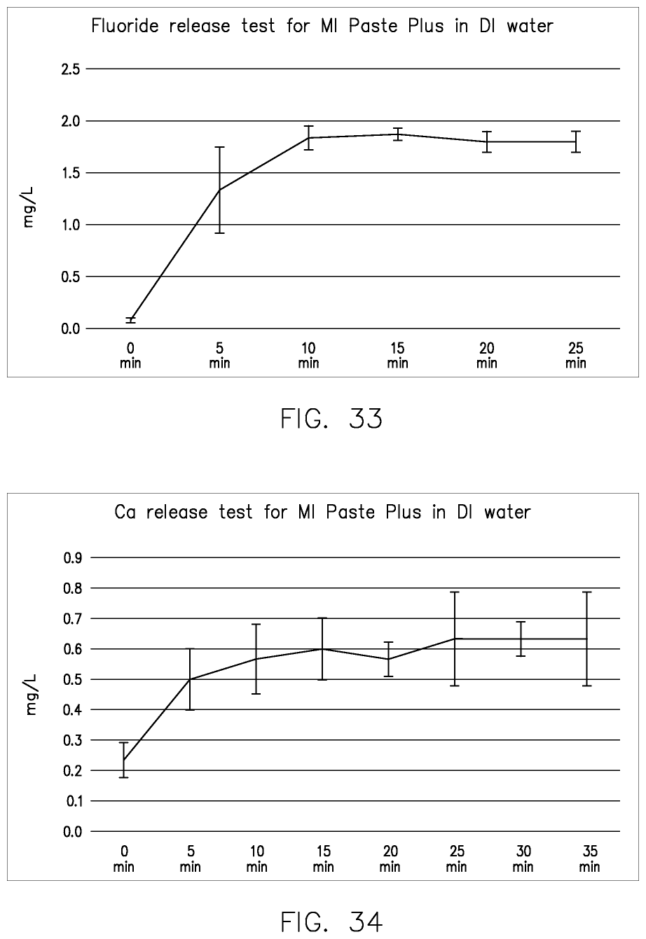

MI Paste Plus is made by GC America (Aslip, Ill., USA). MI Paste Plus contains pure water, glycerol, CPP-ACP, d-sorbitol, CMC-Na, propylene glycol, silicon dioxide, titanium dioxide, 2% xylitol, phosphoric acid, sodium fluoride, flavoring, sodium saccharin, ethyl p-hydroxybenzoate, propyl p-hydroxybenzoate, and butyl p-hydroxybenzoate. It is available in mint, vanilla, strawberry, melon, or tutti-frutti flavors. MI Paste Plus contains 0.2% (900 ppm) fluoride and the active ingredient for remineralization is CPP-ACP (RECALDENT™) CPP-ACP=casein phosphopeptide−amorphous calcium phosphate. The results for the fluoride release test are shown in the graph in FIG. 33.

### Example 40

Calcium release test for MI Paste Plus in DI water:

Steps:

1. Weigh 0.12 g of MI paste plus (this paste has more fluoride than the MI Paste) on a weighing paper.

2. Put 60 ml DI water in a 250 ml glass beaker.

3. Adjust stir plate speed at 250 RPM at room temperature.

4. Put a stir bar in the middle of the glass beaker.

5. Add 0.12 g of MI paste plus to the 60 ml DI water.

6. Take measurements every 5 minutes by taking 1 ml sample and add 20 Microliter Calcium ISA to it.

7. Add 1 ml DI water to the glass beaker every 5 minutes to keep the 60 ml soln. constant.

| Time | Sample 1 | Sample 2 | Sample 3 | AVERAGE | STDEV |
|---|---|---|---|---|---|
| 0 min | 0.2 | 0.3 | 0.2 | 0.2 | 0.1 |
| 5 min | 0.5 | 0.6 | 0.4 | 0.5 | 0.1 |
| 10 min | 0.7 | 0.5 | 0.5 | 0.6 | 0.1 |
| 15 min | 0.7 | 0.5 | 0.6 | 0.6 | 0.1 |
| 20 min | 0.5 | 0.6 | 0.6 | 0.6 | 0.1 |
| 25 min | 0.8 | 0.6 | 0.5 | 0.6 | 0.2 |
| 30 min | 0.6 | 0.7 | 0.6 | 0.6 | 0.1 |
| 35 min | 0.8 | 0.6 | 0.5 | 0.6 | 0.2 |

Reading at 0 min Background DI water only.

The results are shown in the graph in FIG. 34.

### Example 41

Calcium release test for MI Paste in DI water:

Reading at 0 min Background DI water only.

| Time | Sample 1 | Sample 2 | Sample 3 | AVERAGE | STDEV |
|---|---|---|---|---|---|
| 0 min | 0.2 | 0.2 | 0.3 | 0.2 | 0.1 |
| 5 min | 1.2 | 1.1 | 1.4 | 1.2 | 0.2 |
| 10 min | 1.3 | 1.3 | 1.6 | 1.4 | 0.2 |
| 15 min | 1.2 | 1.1 | 1.8 | 1.4 | 0.4 |
| 20 min | 1 | 1.3 | 1.6 | 1.3 | 0.3 |
| 25 min | 1 | 1.2 | 1.6 | 1.3 | 0.3 |
| 30 min | 1.1 | 1.1 | 1.5 | 1.2 | 0.2 |

MI Paste is made by GC America (Aslip, Ill., USA). MI Paste contains RECALDENT™ (CPP-ACP); Casein Phosphopeptides (CPP), which are natural occurring molecules which are able to release calcium and phosphate ions and stabilize Amorphous Calcium Phosphate (ACP). RECALDENT™ (CPP-ACP) is milk-derived having a lactose content less than 0.01%. More specifically, MI Paste contains pure water, glycerol, CPP-ACP, D-sorbitol, CMC-Na, propylene glycol, silicon dioxide, titanium dioxide, 2% xylitol, phosphoric acid, flavoring, zinc oxide, sodium saccharin, ethyl p-hydroxybenzoate, magnesium oxide, guar gum, propyl p-hydroxybenzoate, and butyl p-hydroxybenzoate. It is available in mint, vanilla, strawberry, melon, or tutti-frutti flavors.

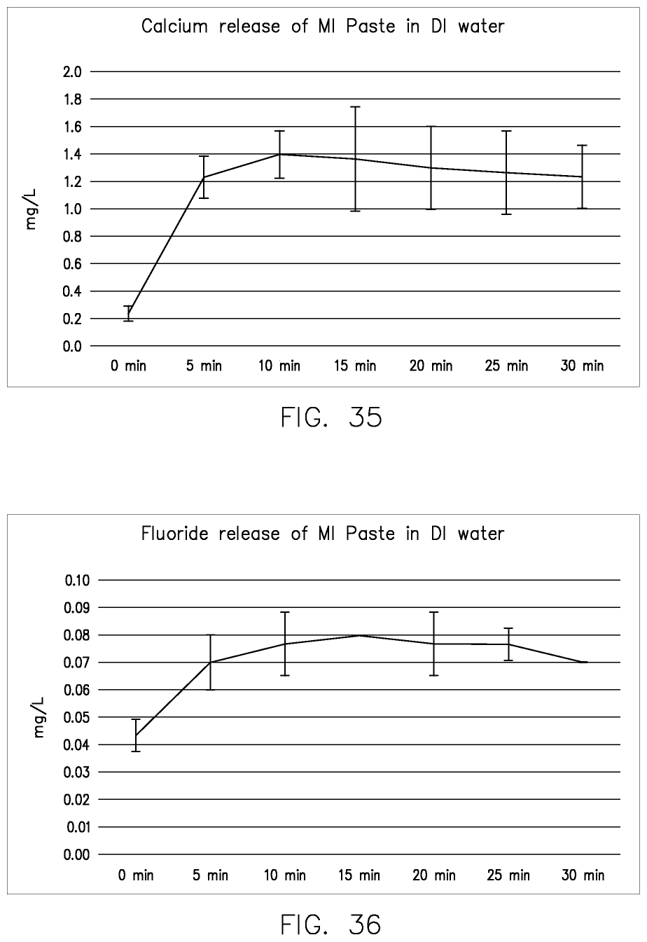

The results are shown in the graph in FIG. 35. It was noted that the MI Paste Plus has less calcium release than the MI Paste. One possible reason for this observation is that the MI Paste Plus has more fluoride in it, which may interfere with calcium release.

### Example 42

Fluoride release test for MI paste in DI water:

Reading at 0 min Background DI water only.

| Time | Sample 1 | Sample 2 | Sample 3 | AVERAGE | STDEV |
|---|---|---|---|---|---|
| 0 min | 0.05 | 0.04 | 0.04 | 0.04 | 0.01 |
| 5 min | 0.07 | 0.08 | 0.06 | 0.07 | 0.01 |
| 10 min | 0.09 | 0.07 | 0.07 | 0.08 | 0.01 |
| 15 min | 0.08 | 0.08 | 0.08 | 0.08 | 0.00 |
| 20 min | 0.07 | 0.09 | 0.07 | 0.08 | 0.01 |
| 25 min | 0.08 | 0.07 | 0.08 | 0.08 | 0.01 |
| 30 min | 0.07 | 0.07 | 0.07 | 0.07 | 0.00 |

The results are shown in the graph in FIG. 36.

### Example 43

Theoretically calculation of Fluoride amount in 0.12 g MI Paste Plus

1. MI paste plus has 0.2% w/w (900 ppm or 900 mg/L Fluoride)

2. This means 0.2 g of Fluoride/100 g of MI paste plus

3. 200 mg . . . 100 g
X . . . 0.12 g

4. X=(200×0.12)/100=0.24 mg

5. 0.24 mg . . . 60 ml
X . . . 1000 ml

6. X=(0.24×1000)/60=4 mg/L

Theoretically, 0.12 g of MI paste plus has F release of 4 mg/L.

### Example 44

Calcium release test for MI Paste in Artificial Saliva:

Preparation of 200 ml artificial saliva:
1. 200 ml DI water
2. 0.0442 g CaCl2
3. 0.0244 g KH2PO4
4. 0.9532 g HEPES
5. 1.9386 g KCl

Then, add a few drops of 1M KOH to adjust PH at 7.

Artificial saliva was used instead of DI water

Add 1 ml of artificial saliva every 5 min to the 60 ml soln to keep it constant.

Reading at 0 min Background artificial saliva only.

| Time | Sample 1 | Sample 2 | Sample 3 | AVERAGE | STDEV |
|---|---|---|---|---|---|
| 0 min | 25 | 24.6 | 24.8 | 24.8 | 0.2 |
| 5 min | 23.7 | 23.2 | 23.4 | 23.4 | 0.3 |
| 10 min | 21.8 | 22.1 | 23.7 | 22.5 | 1.0 |
| 15 min | 21.9 | 20.4 | 22.9 | 21.7 | 1.3 |
| 20 min | 22.7 | 22.6 | 21.6 | 22.3 | 0.6 |
| 25 min | 21.4 | 21 | 23.3 | 21.9 | 1.2 |
| 30 min | 23 | 22.3 | 23.8 | 23.0 | 0.8 |

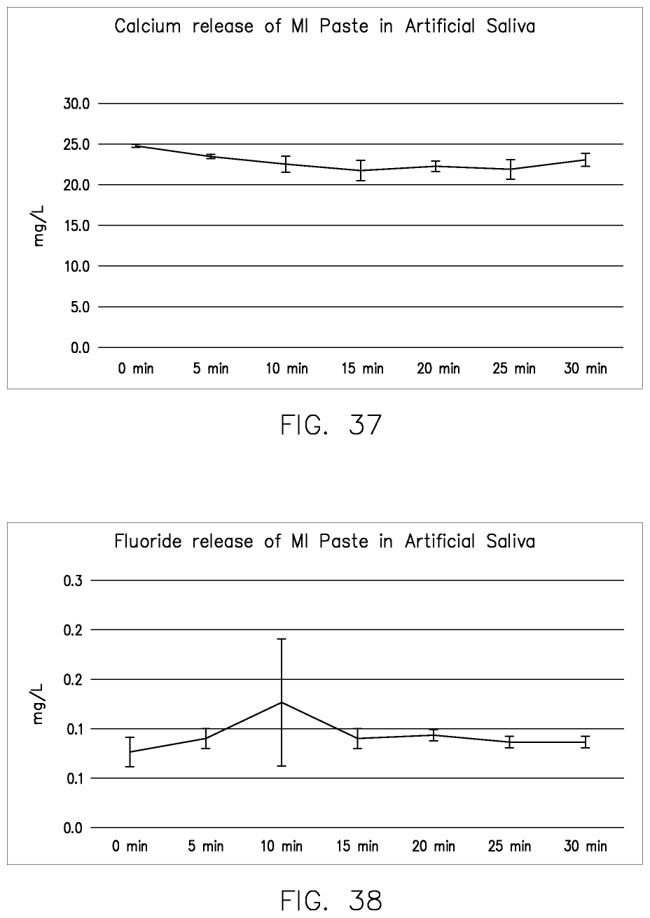

The results are shown in the graph in FIG. 37.

### Example 45

Fluoride release test for MI Paste in Artificial Saliva:

Add 1 ml artificial saliva every 5 min to the 60 ml soln. to keep it constant. The artificial saliva was used in place of DI water.

Reading at 0 min Background artificial saliva only.

The results are shown in the graph in FIG. 38

### Example 46

Calcium release test for MI Paste Plus in Artificial Saliva:

Add 1 ml of artificial saliva every 5 min to the 60 ml soln. to keep it constant. Artificial saliva was used instead of DI water.

Reading at 0 min Background artificial saliva only.

| Time | Sample 1 | Sample 2 | Sample 3 | AVERAGE | STDEV |
|---|---|---|---|---|---|
| 0 min | 25.1 | 25 | 25.2 | 25.1 | 0.1 |
| 5 min | 22.7 | 23.7 | 22.1 | 22.8 | 0.8 |
| 10 min | 20 | 22.7 | 22.5 | 21.7 | 1.5 |
| 15 min | 23.2 | 22.8 | 23.1 | 23.0 | 0.2 |
| 20 min | 23 | 23.4 | 22.6 | 23.0 | 0.4 |
| 25 min | 23.7 | 21.6 | 20 | 21.8 | 1.9 |
| 30 min | 23.5 | 22.2 | 18.8 | 21.5 | 2.4 |

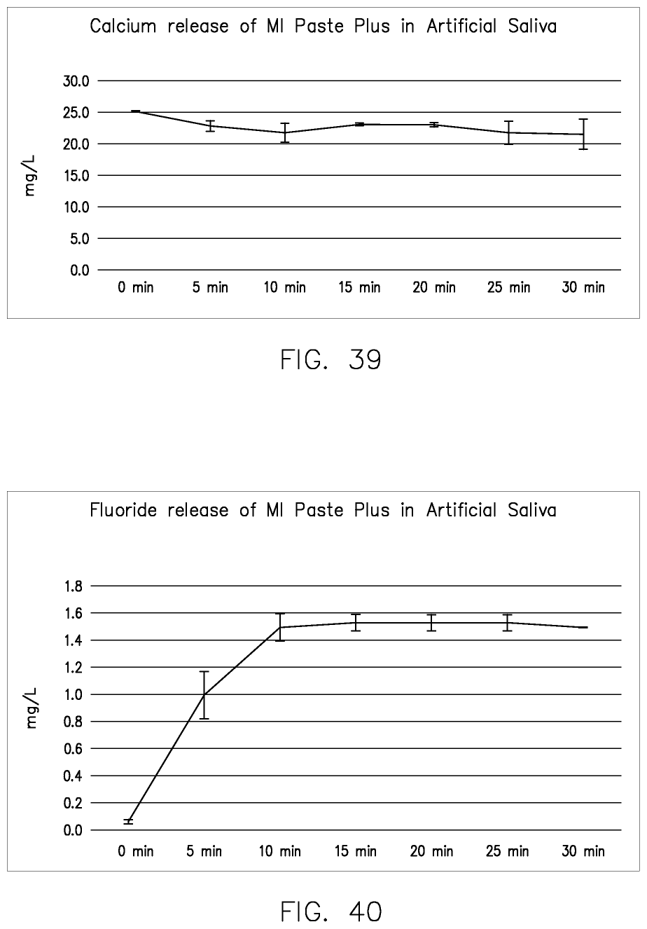

The results are shown in the graph in FIG. 39.

### Example 47

Fluoride release test for MI Paste Plus in Artificial Saliva:

Add 1 ml artificial saliva every 5 min to the 60 ml soln. to keep it constant. Artificial saliva was used instead of DI water.

Reading at 0 min Background artificial saliva only.

| Time | Sample 1 | Sample 2 | Sample 3 | AVERAGE | STDEV |
|---|---|---|---|---|---|
| 0 min | 0.08 | 0.06 | 0.05 | 0.1 | 0.0 |
| 5 min | 0.9 | 0.9 | 1.2 | 1.0 | 0.2 |
| 10 min | 1.5 | 1.4 | 1.6 | 1.5 | 0.1 |
| 15 min | 1.6 | 1.5 | 1.5 | 1.5 | 0.1 |
| 20 min | 1.6 | 1.5 | 1.5 | 1.5 | 0.1 |
| 25 min | 1.6 | 1.5 | 1.5 | 1.5 | 0.1 |
| 30 min | 1.5 | 1.5 | 1.5 | 1.5 | 0.0 |

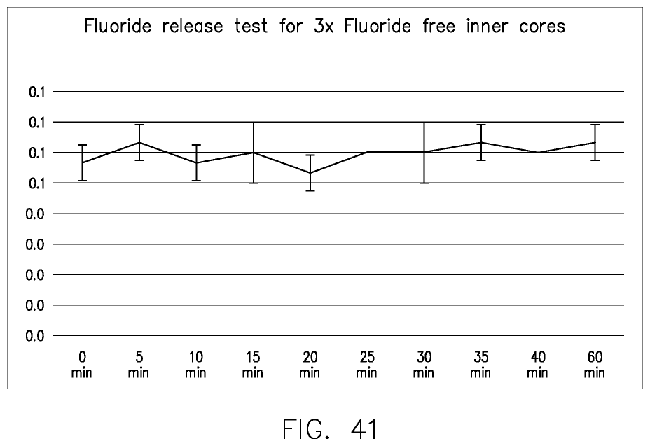

The results are shown in the graph in FIG. 40.

### Example 48

Determination of Organic Matcha Powder's Particle size:

1 g of Organic Matcha powder was put on top of a chain of sieves sized from top to bottom: 1 mm, 500 Mm, 250 Mm, 125 Mm, 53 Mm and 25 Mm.

Results:

1^{st} Trial

1. 100% of Matcha powder passed through 1.00 mm sieve
2. 100% of Matcha powder passed through 500 Mm sieve
3. 100% of Matcha powder passed through 250 Mm sieve
4. 0.0392 g of Matcha powder>125 Mm sieve
5. 0.292 g of Matcha powder>53 Mm sieve
6. 0.362 g of Matcha powder>25 mm sieve

2^{nd} Trial

1. All matcha powder passed through sieves sized 1.00 mm, 500 Mm and 250 Mm.
2. 0.06 g of Matcha powder>125 Mm sieve
3. 0.35 g Matcha powder>53 Mm sieve
4. 0.28 g Matcha powder>25 Mm sieve

In both trials, we lost about 0.3 g out of 1 g Organic Matcha powder used in particle size test. Our interpretation of what happened is part of Matcha powder was stuck within filters of different sieves used in particle size determination test.

### Example 49

Measuring particle size of ground Organic Matcha Powder:

1^{st} Trial: Testing 1 g of Matcha powder
0.02 g>125 Mm sieve
0.45 g>53 Mm sieve
0.35 g>25 Mm sieve

2^{nd} Trial: Testing 1 g of Matcha powder
0.04 g>125 Mm sieve
0.42 g>53 Mm sieve
0.4 g>25 Mm sieve

### Example 50

Fluoride release test for 3× Fluoride free inner cores:

Inner cores were made.

Reading at 0 min Background DI water only

| Time | Sample 1 | Sample 2 | Sample 3 | AVERAGE | STDEV |
|---|---|---|---|---|---|
| 0 min | 0.05 | 0.06 | 0.06 | 0.1 | 0.0 |
| 5 min | 0.07 | 0.06 | 0.06 | 0.1 | 0.0 |
| 10 min | 0.06 | 0.06 | 0.05 | 0.1 | 0.0 |
| 15 min | 0.06 | 0.07 | 0.05 | 0.1 | 0.0 |
| 20 min | 0.06 | 0.05 | 0.05 | 0.1 | 0.0 |
| 25 min | 0.06 | 0.06 | 0.06 | 0.1 | 0.0 |
| 30 min | 0.05 | 0.07 | 0.06 | 0.1 | 0.0 |
| 35 min | 0.06 | 0.07 | 0.06 | 0.1 | 0.0 |
| 40 min | 0.06 | 0.06 | 0.06 | 0.1 | 0.0 |
| 60 min | 0.07 | 0.06 | 0.06 | 0.1 | 0.0 |

The results are shown in the graph in FIG. 41. The formulation used in this release test is listed in Table 43. It has three times the amount of calcium and phosphate in it and the fluoride release was from the Matcha.

### Example 51

Re-measuring particle size of regular and ground Organic Matcha powder:

We weighed sieves before start testing particle size and weighed them again after testing to include remaining powder that stuck in filters of different sieves.

Light shaking has been performed.

Metallic spatula has been used to spread Matcha powder along each sieve's filter.

The chain of sieves has been perfectly sealed from top and bottom to avoid escaping of powder particles.

Organic Matcha Powder: (1 g has been tested)

0.39 g>125 Mm sieve

0.37 g>53 Mm sieve

0.18 g>25 Mm sieve

0.01 g<25 Mm sieve

Ground Organic Matcha Powder: (1 g has been tested)

0.344 g>125 Mm sieve

0.42 g>53 Mm sieve

0.13 g>25 Mm sieve

0.005 g<25 Mm sieve

Regarding the last two values related to sieve size 25 Mm, the first value is for Matcha powder particles larger than 25 Mm but smaller than 53 Mm. In other words, this value represents Matcha that passed through sieve 53 Mm and did not pass through sieve sized 25 Mm.

The second-last-value represents Matcha powder particles smaller than 25 Mm. A very small amount of Matcha particles were able to pass through the filter of sieve sized 25 Mm. This procedure was done to reduce or obtain the desired particle size of the Matcha.

Before measuring particle size, we weighed the clean weighing paper that was put under sieve sized 25 Mm. After testing particle size, we weighed the weighing paper again to determine how much Matcha powder particles were able to pass through sieve 25 Mm.

### REFERENCES

1. Tea Association of the U.S.A. Inc. Wathen J. Tea Fact Sheet—2018-2019. Downloaded from http://www.teausa.com/14655/tea-fact-sheet
2. Imfeld T. Dental erosion. Definition, classification and links. Eur J Oral Sci 1996;
3. Schroeder P L, Filler S, Ramirez B, Lazarchik D A, Vaezi M F, Richter J E. Dental erosion and acid reflux disease. Ann Intern Med 1995; 122:809-815.
4. Valena V, Young W G. Dental erosion patterns from intrinsic acid regurgitation and vomiting. Aust Dent J 2002; 47:106-115.
5. Barron R P, Carmichael R P, Marcon M A, Sandor G K. Dental erosion in gastroesophageal reflux disease. J Can Dent Assoc 2003; 69:84-89.
6. Pace F, Pallotta S, Tonini M, Vakil N, Bianchi Porro G. Systematic review: gastro-oesophageal reflux disease and dental lesions. Aliment Pharm Ther 2008; 27:1179-1186.
7. National Eating Disorders Association. Statistics: Eating disorders and their precursors http://www.nationaleatingdisorders.org/uploads/statistics tmp.pdf. Downloaded Nov. 5, 2012.
8. Zero D T, Lussi A. Erosion—chemical and biological factor of importance to the dental practitioner. Int Dent J 2005; 55:285-290.
9. Singhal K, Raj N, Gupta K, Singh S. Probable benefits of green tea with genetic implications. J Oral Maxillofac Pathol. 2017; 21(1): 107-114.
10. Sesso H D, Gaziano J M, Buring J E, Hennekens C H. Coffee and tea intake and the risk of myocardial infarction. Am J Epidemiol. 1999; 149(2):162-167.
11. Arab L, Liu W, Elashoff D. Green and Black Tea Consumption and Risk of Stroke. A Meta-Analysis. Stroke. 2009; 40(5):1786-1792.
12. Miller P E, Zhao D, Frazier-Wood A C, Michos E D, Averill M, Sandford V, Burke G L, et al. Associations of coffee, tea and caffeine intake with coronary artery calcification and cardiovascular events. Am J Med. 2016; 130(2): 188-197.
13. Zheng W, Doyle T J, Kushi L H, Sellers T A, Hong C P, Folsom A R. Tea consumption and cancer incidence in a prospective cohort study of postmenopausal women. Am J Epidemiol. 1996; 144(2):175-182.
14. Yang C S, Wang Z Y. Tea and cancer. J Natl Cancer Inst. 1993; 85(13):1038-1049.
15. Trevisanato S I, Kim Y I. Tea and health. Nutr Rev. 2000; 58(1):1-10.
16. U.S. Department of Agriculture. USDA National Fluoride Database of Selected Beverages and Foods Release 2. December 2005. Downloaded from https://www.ars.usda.gov/ARSUserFiles/80400525/Data/Fluoride/F02.pdf
17. Zhang L, Li Q, Ma L F, Ruan J Y. Characterization of fluoride uptake by roots of tea plants (Camellia sinensis (L.) O. Kuntze). Plant and Soil. 2013; 366(1-2):659-669.
18. Centers for Disease Control and Prevention. Achievements in Public Health, 1900-1999: Fluoridation of Drinking Water to Prevent Dental Caries. MMWR Morb Mortal Wkly Rep. 1999:48(41):933-940.
19. ten Cate J M. Current concepts on the theories of the mechanism of action of fluoride. Acta Odontol Scand. 1999; 57(6):325-329.
20. Carey C M. Focus on fluorides: update on the use of fluoride for the prevention of dental caries. J Evid Based Dent Pract. 2014; 14(Suppl):95-102.
21. DenBesten P, Li W. Chronic fluoride toxicity: dental fluorosis. Monogr Oral Sci. 2011; 22: 81-96.
22. Pendrys D G. Risk of enamel fluorosis in nonfluoridated and optimally fluoridated populations: considerations for the dental professional. J Am Dent Assoc. 2000; 131(6):746-755.
23. Rosen S, Elvin-Lewis M, Beck F M, Beck E X. Anticariogenic effects of tea in rats. J Dent Res. 1984; 63(5):658-660.
24. Simpson A, Shaw L, Smith A J. The bio-availability of fluoride from black tea. J Dent. 2001; 29(1):15-21.
25. Chan L, Mehra A, Saikat S, Lynch P. Human exposure assessment of fluoride from tea (Camellia sinensis L.): A UK based issue? Food Res Int. 2013; 51(2):564-570.
26. Cao J, Zhao Y, Li Y, Deng H J, Yi J, Liu J W. Fluoride levels in various black tea commodities: measurement and safety evaluation. Food Chem Toxicol. 2006; 44(7):1131-1137.
27. Centers for Disease Control and Prevention. Youth Risk Behavior Surveillance—United States 2015. MMWR Morb Mortal Wkly Rep. 2016:65(6):1-174.
28. American Dental Association Facebook. If you really love kombucha, it may not be as great for your teeth. Mar. 29, 2019. Downloaded from https://www.facebook.com/AmericanDentalAssociation/posts/10155856033207167
29. Waugh D T, Potter W, Limeback H, Godfrey M. Risk Assessment of Fluoride Intake from Tea in the Republic of Ireland and its Implications for Public Health and Water Fluoridation. Int J Environ Res Public Health. 2016; 13(3) pii: E259.
30. U.S. Department of Health and Human Services Federal Panel on Community Water Fluoridation. U.S. Public Health Service Recommendation for Fluoride Concentration in Drinking Water for the Prevention of Dental Caries. Public Health Rep. 2015; 130:318-331.
31. World Health Organization. Fluoride in drinking water: Background document for development of WHO guidelines for drinking water. 2004 Downloaded from https://www.who.int/water_sanitation_health/dwq/chemicals/fluoride.pdf
32. Linhares D P S, Garcia P V, Amaral L, Ferreira T, Dos Santos Rodrigues A. Safety Evaluation of Fluoride Content in Tea Infusions Consumed in the Azores—a Volcanic Region with Water Springs naturally Enriched in Fluoride. Biol Trace Elem Res. 2017; 179(1):158-164. Publications by Investigators in Scientific Journals related to this Disclosure:
33. Dehghan M, Ozorio J E V, Chanin S, Tantbirojn D, Versluis A, Garcia-Godoy F. Investigating enamel loss when brushing with an anti-erosive toothpaste after an acidic episode. Accepted for publication General Dentistry 2016.
34. Dehghan M, Tantbirojn D, Kymer-Davis E, Stewart C W, Zhang Y H, Versluis A, Garcia-Godoy F. Neutralizing saliva pH by mouthwashes after an acidic challenge. Journal of Investigative and Clinical Dentistry, November 30 doi: 10.1111/jicd.12198.
35. Fitzhugh A C, Dehghan M, Tantbirojn D, Simon, Effectiveness of a Neutralizing Mouthwash in rehardening Softened Enamel. Journal Cosmetic Dentistry 2014:30(3)Fall:72-79, 2014.
36. Dehghan M, Stanely P J, Tantbirojn D, Versluis A. “Investigation of Treatment Options to Minimize The effects of Acid Erosion on Enamel.” Gen Dent 2014 July/August; 62(4): e30-e33.
37. Dehghan M, Alex Fitzhugh, Tantbirojn D, Versluis A. “Hardness Recovery of Softened Enamel after Neutralizing and Fluoride rinse”. J Dent Research 2013; 91 (Spec Issue): Abstract 174900
38. Dehghan M, Stanley P J, Tantbijorn D, Versluis A. Investigation of Treatments to Improve Hardness Recovery of Softened Enamel. J Dent Research 2012; 91 (Spec Issue): Abstract 330.

Every document cited herein, including any cross referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

It will be apparent to those skilled in the art that various modifications and variations can be made to various embodiments described herein without departing from the spirit or scope of the teachings herein. Thus, it is intended that various embodiments cover other modifications and variations of various embodiments within the scope of the present teachings.

1-2. (canceled)

3. An oral composition for neutralizing saliva and re-hardening tooth enamel, the oral composition comprising an alkalinizing agent; a re-mineralizing agent comprising Matcha green tea; a base; a plasticizer; and a sugar alcohol, wherein the oral composition is in the form of a lozenge or a lollipop.

4. The composition according to claim 3, wherein the re-mineralizing agent comprises Matcha green tea, the base comprises crosslinked polyvinylpyrrolidone (PVP), the plasticizer comprises glycerin, and the sugar alcohol comprises xylitol.

5. (canceled)

6. The composition according to claim 4, wherein the crosslinked PVP is in an amount of from about 1% to about 50% of the composition, the xylitol is in an amount of from about 20% to about 95% of the composition, Matcha green tea is in an amount of from about 2.25% to about 6.65% and the glycerin is in an amount from about 0.1% to about 1% of the composition.

7. The composition according to claim 3, wherein the alkalinizing agent comprises eucalyptus, licorice root, or arginine.

8. The composition according to claim 3, wherein the alkalinizing agent comprises sodium bicarbonate, or potassium bicarbonate, and the re-mineralizing agent comprises Matcha green tea and calcium chloride, or potassium monophosphate.

9. (canceled)

10. The composition according to claim 8, wherein the calcium chloride is in an amount of from about 0.01% to about 0.5% of the composition, the potassium monophosphate is in an amount of from about 0.01% to about 0.5% of the composition, and/or Matcha green tea is in an amount of from about 2.25% to about 6.65% of the composition.

11. (canceled)

12. The composition according to claim 10, wherein the composition raises a user's saliva pH from about 7 to about 10.

13. The composition according to claim 3, wherein the composition releases fluoride in an amount from about 0.9 mg/L to about 3.55 mg/L over a period of about 30 minutes.

14. (canceled)

15. The composition according to claim 8, wherein the Matcha green tea is ground and the composition (i) releases fluoride in an amount from about 1.37 mg/L to about 5.1 mg/L; or (ii) releases calcium in an amount from about 0.26 mg/L to about 1.8 mg/L.

16. A multi-layer lozenge or lollipop for neutralizing saliva and re-hardening tooth enamel, the lozenge or lollipop comprising an alkalinizing agent; a re-mineralizing agent comprising Matcha green tea; a base; a plasticizer; and a sugar alcohol, wherein the lozenge or lollipop upon oral administration raises the saliva pH from about 7 to about 10 in about 1 minute.

17. The multi-layer lozenge or lollipop according to claim 16, wherein the base comprises crosslinked polyvinylpyrrolidone (PVP), the plasticizer comprises glycerin, and the sugar alcohol comprises xylitol.

18. The multi-layer lozenge or lollipop according to claim 16, wherein the crosslinked PVP is Kollidon® Cl, and the xylitol and crosslinked PVP are in a 5:1 ratio.

19. The multi-layer lozenge or lollipop according to claim 16, wherein the crosslinked PVP is in an amount of from about 1% to about 50% of the lozenge or lollipop, the xylitol is in an amount of from about 20% to about 95% of the lozenge or lollipop, the Matcha green tea is in an amount from about 2.25% to about 6.65% of the composition and the glycerin is in an amount of from about 0.1% to about 1% of the lozenge or lollipop.

20. The multi-layer lozenge or lollipop according to claim 16, wherein the alkalinizing agent comprises (i) eucalyptus, (ii) licorice root, or (iii) L-arginine.

21. (canceled)

22. The multi-layer lozenge or lollipop according to claim 16, wherein the Matcha green tea comprises organic Matcha green tea, non-organic Matcha green tea or ground organic Matcha green tea.

23-25. (canceled)

26. A method of treating, preventing or reducing dental disease, the method comprising administering an oral composition in the form of a lozenge or lollipop, the composition comprising crosslinked polyvinylpyrrolidine (PVP), xylitol and Matcha green tea to treat, prevent or reduce the dental disease.

27. The method of claim 26, wherein the dental disease comprises carries, dental decay or acidity of the oral cavity.

28. The method of claim 26, wherein the composition further comprises glycerin, L-arginine, potassium monophosphate, calcium chloride, green tea extract, licorice extract, eucalyptus oil, and casein phosphopeptide.

29. The method of claim 26, wherein the crosslinked PVP is in an amount of from about 1% to about 50% of the lozenge or lollipop, the xylitol is in an amount of from about 20% to about 92% of the lozenge or lollipop, the Matcha green tea is in an amount from about 2.25% to about 6.65% of the composition and the glycerin is in an amount of from about 0.1% to about 1% of the lozenge or lollipop.

30-31. (canceled)

32. The multi-layer lozenge or lollipop according to claim 16, wherein the Matcha green tea has a particle size in the range of less than about 25 mm to greater than about 125 mm.
